(19) United States
(12) Patent Application Publication
(10) Pub. No.: US 2024/0316036 A1
(43) Pub. Date: Sep. 26, 2024

(54) DISULFIDE CONTAINING THERAPEUTICS AND DIAGNOSTICS

(71) Applicant: Singular Genomics Systems, Inc., San Diego, CA (US)

(72) Inventors: Ronald Graham, Carlsbad, CA (US); Surya Adhikari, San Diego, CA (US)

(21) Appl. No.: 18/441,561

(22) Filed: Feb. 14, 2024

Related U.S. Application Data
(60) Provisional application No. 63/446,620, filed on Feb. 17, 2023.

(51) Int. Cl. A61K 31/4745; A61K 47/68; C07K 16/18

(52) U.S. Cl. CPC A61K 31/4745; A61K 47/68037; C07K 16/18

(57) ABSTRACT

Disclosed herein, inter alia, are therapeutic and theragnostic compounds and methods of use thereof.

### CROSS-REFERENCES TO RELATED APPLICATIONS

This application claims the benefit of U.S. Provisional Application No. 63/446,620, filed Feb. 17, 2023, which is incorporated herein by reference in its entirety and for all purposes.

### BACKGROUND

Prodrugs refer to molecules with minimal measurable biological activity that may be metabolized into a biologically active molecule upon enzymatic, chemical, or a combination of both enzymatic and chemical conversion in vivo. As a result, prodrugs are enticing therapeutics due to the ability to selectively activate the compounds as needed. For example, the antibacterial agent Sultamicillin® includes an ampicillin moiety linked to a β-lactamase inhibitor with a diester bond, that is hydrolyzed in vivo to release the two compounds to treat bacterial infections such as β-lactamase producing bacteria (e.g., ampicillin-resistant H. influenzae) of the upper and lower respiratory tract. Redox responsive disulfide bonds are being exploited in this field, due to an abundance of glutathione, thioredoxin, and thioredoxin reductase in cells and organisms. However, common approaches to attaching disulfide moieties to bioactive agents suffers from bulky steric hinderance and slow reaction kinetics. Thus, there is a need in in the art to improve the activation of bioactive agents. Disclosed herein, inter alia, are solutions to these and other problems in the art.

### BRIEF SUMMARY

In an aspect is provided a prodrug having the formula

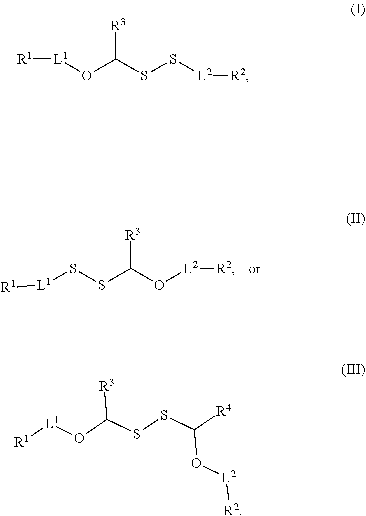

R¹ is a first drug moiety. R² is a second drug moiety, protein moiety, nucleic acid moiety, a polymer moiety, nanoparticle, detectable agent, or a tropic cell. R³ and R⁴ are independently hydrogen, halogen, —CCl₃, —CBr₃, —CF₃, —CI₃,

“\*MERGEFORMAT\*MERGEFORMAT —CHCl₂, —CHBr₂, —CHF₂, —CHI₂, —CH₂Cl, —CH₂Br, —CH₂F, —CH₂I, —CN, —OH, —NH₂, —COOH,

“\*MERGEFORMAT\*MERGEFORMAT —CONH₂, —NO₂, —SH, —SO₃H, —SO₄H, —SO₂NH₂, —NHNH₂, —ONH₂, —NHC(O)NHNH₂,

“\*MERGEFORMAT\*MERGEFORMAT —NHC(O)NH₂, —NHSO₂H, —NHC(O)H, —NHC(O)OH, —NHOH, —OCCl₃, —OCF₃, —OCBr₃, —OCI₃,

“\*MERGEFORMAT\*MERGEFORMAT —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. L¹ and L² are independently covalent linkers.

In an aspect is provided a method of diagnosing, monitoring, or treating a pathological condition, including administering a therapeutically effective amount of a prodrug of formula (I), (II), or (III) to a subject, where the effective amount is sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition).

### BRIEF DESCRIPTION OF THE DRAWINGS

FIGS. 1A-1B. FIG. 1A illustrates a therapeutic prodrug containing a chemically activatable groups (i.e., a cleavable linking cap, CLC) is administered to a subject. Due to an abundance of glutathione, thioredoxin, cysteine, and thioredoxin reductase in diseased cells and organisms (depicted as shaded circles), the CLC is cleaved and the activated therapeutic moiety is delivered to the diseased cells. Alternatively, a therapeutic conjugated to a secondary agent (e.g., antibody) via a cleavable linker moiety (CLM), as illustrated in FIG. 1B, may be administered to a subject and the antibody binds to an overexpressed protein associated with a diseased cell (depicted as shaded ovals). The CLM is subsequently cleaved at or around the diseased cell, enabling targeted delivery of a therapeutic agent.

FIGS. 2A-2B. FIG. 2A exemplifies FDA approved antibody-drug conjugates containing disulfide linkers. FIG. 2B shows an application of the CLM as described herein to commercially available antibody-drug conjugates shown in FIG. 2A.

FIGS. 3A-3B. FIG. 3A presents cleavage kinetics of two CLMs described herein, CLM #1 and CLM #2 at 55° C. FIG. 3B provides the cleavage half time of CLM #1 and CLM #2. Cleavage of the disulfide group from both CLMs was performed at 65° C. and in the presence of 1 mM and 10 mM of reducing agent, tris(3-hydroxypropyl)phosphine (THPP).

FIG. 4 shows the ylide and related resonance structures for different disulfide-containing CLMs. The greater number of resonant structures is a reflection of the greater electron delocalization, which lowers the potential energy of the thioaldehyde, thereby stabilizing the molecule. A more stable thioaldehyde allows for faster cleavage.

FIGS. 5A-5C. Experimentally derived cleavage kinetics of three compounds bearing different model compounds including cleavable linking moieties. FIG. 5A reports that the cleavage kinetics of CLM #3 are improved relative to CLM #1 and CLM #2. FIG. 5B reports on the average cleavage halftime for CLM #1, #2, and #3, where CLM #1 is measured at an elevated temperature (65° C.), and CLM #2 and CLM #3 are measured at a lower temperature (55° C.) with identical concentrations of a cleaving agent, 1 mM THPP. FIG. 5C reports on the average cleavage halftime for CLM #2, #3, #45, and #26 under identical cleavage conditions, 0.1 mM THPP at 55° C.

### DETAILED DESCRIPTION

The aspects and embodiments described herein relate to composition of therapeutic prodrugs with a chemically activable group (i.e., cleavable linking moiety with disulfide moiety). Particular aspects and embodiments described herein relate to the pharmaceutical composition of the prodrugs described herein with a pharmaceutically acceptable excipient. As described herein, the methods and compositions of this disclosure have many advantages, including improved cleavage reaction kinetics and versatile applications with targeted therapy (e.g., antibody-drug conjugates, nanoparticles, and theranostics).

### I. Definitions

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

The abbreviations used herein have their conventional meaning within the chemical and biological arts. The chemical structures and formulae set forth herein are constructed according to the standard rules of chemical valency known in the chemical arts.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by,

“\*MERGEFORMAT\*MERGEFORMAT —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to:

“\*MERGEFORMAT\*MERGEFORMAT —CH₂—CH₂—O—CH₃, —CH₂—CH₂—NH—CH₃, —CH₂—CH₂—N(CH₃)—CH₃, —CH₂—S—CH₂—CH₃,

“\*MERGEFORMAT\*MERGEFORMAT —CH₂—S—CH₂, —S(O)—CH₃, —CH₂—CH₂—S(O)₂—CH₃, —CH═CHO—CH₃, —Si(CH₃)₃,

“\*MERGEFORMAT\*MERGEFORMAT —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as

“\*MERGEFORMAT\*MERGEFORMAT —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. In embodiments, a bicyclic or multicyclic cycloalkyl ring system refers to multiple rings fused together or multiple spirocyclic rings wherein at least one of the fused or spirocyclic rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, a bicyclic or multicyclic cycloalkenyl ring system refers to multiple rings fused together or multiple spirocyclic rings wherein at least one of the fused or spirocyclic rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. In embodiments, a bicyclic or multicyclic heterocycloalkyl ring system refers to multiple rings fused together or multiple spirocyclic rings wherein at least one of the fused or spirocyclic rings is a heterocycloalkyl ring and wherein the multiple rings are attached to the parent molecular moiety through any atom contained within a heterocycloalkyl ring of the multiple rings.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. In embodiments, a bicyclic or multicyclic cycloalkyl ring system refers to multiple rings fused together or multiple spirocyclic rings wherein at least one of the fused or spirocyclic rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, a bicyclic or multicyclic cycloalkenyl ring system refers to multiple rings fused together or multiple spirocyclic rings wherein at least one of the fused or spirocyclic rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or multicyclic heterocycloalkyl ring system. In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted, and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

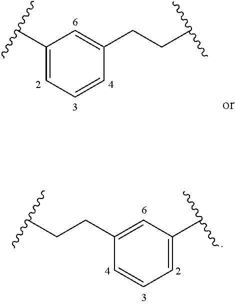 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃,

“*\MERGEFORMAT\*MERGEFORMAT —CF₃, —CCl₃, —CBr₃, —CI₃, —CN, —CHO, —OH, —NH₂, —COOH, —CONH₂, —NO₂, —SH, —SO₂CH₃,

“*\MERGEFORMAT\*MERGEFORMAT —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′,

“\*MERGEFORMAT\*MERGEFORMAT —C(O)R′, —CO₂R′, —CONR′R″, —OC(O)NR′R″, —NR″C(O)R′, —NR′—C(O)NR″R′″,

“\*MERGEFORMAT\*MERGEFORMAT —NR″C(O)₂R′, —NR—C(NR′R″R′″)═NR″″, —NR—C(NR′R″)═NR′″, —S(O)R′, —S(O)₂R′,

“\*MERGEFORMAT\*MERGEFORMAT —S(O)₂NR′R″, —NRSO₂R′, —NR′NR″R′″, —ONR′R″, —NR′C(O)NR″NR′″R″″, —CN, —NO₂,

“\*MERGEFORMAT\*MERGEFORMAT —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen,

“\*MERGEFORMAT\*MERGEFORMAT —SiR′R″R′″, —OC(O)R′, —C(O)R′, —CO₂R′, —CONR′R″, —OC(O)NR′R″, —NR″C(O)R′,

“\*MERGEFORMAT\*MERGEFORMAT —NR′—C(O)NR″R′″, —NR″C(O)₂R′, —NR—C(NR′R″R′″)═NR″″, —NR—C(NR′R″)═NR′″,

“\*MERGEFORMAT\*MERGEFORMAT —S(O)R′, —S(O)₂R′, —S(O)₂NR′R″, —NRSO₂R′, —NR′NR″R′″, —ONR′R″,

“\*MERGEFORMAT\*MERGEFORMAT —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—,

“\*MERGEFORMAT\*MERGEFORMAT —CRR′—, or a single bond, and q is an integer from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—,

“\*MERGEFORMAT\*MERGEFORMAT —S(O)₂—, —S(O)₂NR′—, or a single bond, and r is an integer from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and/or each substituted or unsubstituted heteroaryl is a substituted or unsubstituted 5 to 6 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted phenylene, and/or each substituted or unsubstituted heteroarylene is a substituted or unsubstituted 5 to 6 membered heteroarylene. In some embodiments, the compound (e.g., nucleotide analogue) is a chemical species set forth in the Examples section, claims, embodiments, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another. It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure, i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure. The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. In embodiments, a detectable moiety is a moiety (e.g., monovalent form) of a detectable agent.

The terms “fluorophore” or “fluorescent agent” or “fluorescent dye” are used interchangeably and refer to a substance, compound, agent (e.g., a detectable agent), or composition (e.g., compound) that can absorb light at one or more wavelengths and re-emit light at one or more longer wavelengths, relative to the one or more wavelengths of absorbed light. Examples of fluorophores that may be included in the compounds and compositions described herein include fluorescent proteins, xanthene derivatives (e.g., fluorescein, rhodamine, Oregon Green®, eosin, or Texas red), cyanine and derivatives (e.g., cyanine, indocarbocyanine, oxacarbocyanine, thiacarbocyanine, or merocyanine), napththalene derivatives (e.g., dansyl or prodan derivatives), coumarin and derivatives, oxadiazole derivatives (e.g., pyridyloxazole, nitrobenzoxadiazole or benzoxadiazole), anthracene derivatives (e.g., anthraquinones, DRAQ5™, DRAQ7™, or CyTrak Orange™), pyrene derivatives (e.g., Cascade Blue® and derivatives), oxazine derivatives (e.g., Nile red, Nile blue, cresyl violet, or oxazine 170), acridine derivatives (e.g., proflavin, acridine orange, acridine yellow), arylmethine derivatives (e.g., auramine, crystal violet, or malachite green), tetrapyrrole derivatives (e.g., porphin, phthalocyanine, bilirubin), CF® dye, DRAQ™, CyTRAK™, BODIPY®, Alexa Fluor®, DyLight™ Fluor, ATTO™, Tracy™, FluoProbes™, Abberior dyes, DY dyes, MegaStokes dyes, Sulfo-Cy® dyes, Seta dyes, SeTau dyes, Square dyes, Quasar™ dyes, Cal Fluor™ dyes, SureLight® dyes, PerCP, phycobilisomes, APC, APCXL, RPE, and/or BPE. A fluorescent moiety is a radical of a fluorescent agent. The emission from the fluorophores can be detected by any number of methods, including but not limited to, fluorescence spectroscopy, fluorescence microscopy, fluorimeters, fluorescent plate readers, infrared scanner analysis, laser scanning confocal microscopy, automated confocal nanoscanning, laser spectrophotometers, fluorescent-activated cell sorters (FACS), image-based analyzers and fluorescent scanners (e.g., gel/membrane scanners). In embodiments, the fluorophore is an aromatic (e.g., polyaromatic) moiety having a conjugated π-electron system. In embodiments, the fluorophore is a fluorescent dye moiety, that is, a monovalent fluorophore.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, 99Mo, ¹⁰⁵Pd, ¹⁰⁵Rh ¹¹¹Ag, ¹¹¹In ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Examples of detectable agents include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa Fluor® dyes, and cyanine dyes. In embodiments, the detectable moiety is a fluorescent molecule (e.g., acridine dye, cyanine dye, fluorine dye, oxazine dye, phenanthridine dye, or rhodamine dye). In embodiments, the detectable moiety is a fluorescent molecule (e.g., acridine dye, cyanine dye, fluorine dye, oxazine dye, phenanthridine dye, or rhodamine dye). In embodiments, the detectable moiety is a fluorescent moiety or fluorescent dye moiety.

In embodiments, the detectable label is a fluorescent dye. In embodiments, the detectable label is a fluorescent dye capable of exchanging energy with another fluorescent dye (e.g., fluorescence resonance energy transfer (FRET) chromophores).

The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy®3). In embodiments, the cyanine moiety has 5 methine structures (i.e., cyanine 5 or Cy®5). In embodiments, the cyanine moiety has 7 methine structures (i.e., cyanine 7 or Cy®7).

The term “chelator,” “chelating agent,” or “complexing agent” is used in accordance with its plain ordinary meaning and refers to any agent capable of donating a pair of electrons to a metal to form a chelate. An example of a commonly used chelating agent is ethylenediaminetetraacetic acid (EDTA) to chelate calcium, magnesium, lead, and iron. Chelating agents are also found in nuclear medicine for the generation of theranostics and include, but are not limited to, macrocyclic and acyclic chelators. A chelating moiety is a monovalent chelating agent. Examples of chelating agents used for theranostics include, but are not limited to, DOTA (1,4,7,10-tetra-azacyclododecane-1,4,7,10-tetraacetic acid; used for theranostics containing ¹¹¹In, ¹⁷⁷Lu, ^{86/90}Y ²²⁵Ac, ^{44/47}Sc, or ⁶⁸Ga), NOTA (1,4,7-triazacyclononane-1,4,7-triacetic acid; used for theranostics containing ^{67/68}Ga, ⁶⁴Cu, ¹⁷⁷Lu, ^{86/90}Y or ^{212/213}Bi), TETA (1,4,8,11-tetra-azacyclotetradecane-1,4,8,11-tetraacetic acid; used for theranostics containing ⁶⁴Cu), and DTPA (diethylenetriaminepentaacetic acid; used for theranostics containing ¹¹¹In or ¹⁷⁷Lu) (Price et al. Chem Soc Rev. 2014 Jan. 7; 43(1):260-90).

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” “analogue” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Descriptions of compounds (e.g., nucleotide analogues) of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A polypeptide or a cell is “recombinant” when it is artificial or engineered or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

As used herein, the terms “specific”, “specifically”, “specificity”, or the like of a compound refers to the compound's ability to cause a particular action, such as binding, to a particular molecular target with minimal or no action to other proteins in the cell.

The terms “attached,” “bind,” “binding,” and “bound” as used herein are used in accordance with their plain and ordinary meanings and refer to an association between atoms or molecules. The association can be direct or indirect. For example, attached molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

An “antibody” (Ab) is a protein that binds specifically to a particular substance, known as an “antigen” (Ag). An “antibody” or “antigen-binding fragment” is an immunoglobulin that binds a specific “epitope.” The term “antigen-binding site” of an antibody (or simply “antibody portion”), as used herein, refers to one or more fragments of an antibody that retains the ability to specifically bind to an antigen. It has been shown that the antigen-binding function of an antibody can be performed by fragments of a full-length antibody. The term “antibody” encompasses polyclonal, monoclonal, and chimeric antibodies. In nature, antibodies are generally produced by lymphocytes in response to immune challenge, such as by infection or immunization. An antibody may include the entire antibody as well as any antibody fragments capable of binding the antigen or antigenic fragment of interest. Examples of antibody functional fragments include, but are not limited to, include complete antibody molecules, antibody fragments, such as Fv, single chain Fv (scFv), complementarity determining regions (CDRs), VL (light chain variable region), VH (heavy chain variable region), Fab, F(ab′)₂ and any combination of those or any other functional portion of an immunoglobulin peptide capable of binding to target antigen (see, e.g., FUNDAMENTAL IMMUNOLOGY (Paul ed., 4th ed. 2001). Antibodies used herein are immunospecific for, and therefore specifically and selectively bind to, for example, proteins either detected (i.e., biological targets of interest) or used for detection (i.e., probes containing oligonucleotide barcodes) in the methods and devices as described herein.

A “monoclonal antibody” comprises a collection of identical molecules produced by a single B cell lymphocyte clone which are directed against a single antigenic determinant. Monoclonal antibodies can be distinguished from polyclonal antibodies in that monoclonal antibodies must be individually selected whereas polyclonal antibodies are selected in groups of more than one or, in other words, in bulk. Large amounts of monoclonal antibodies can be produced by immortalization of a polyclonal B cell population using hybridoma technology. Each immortalized B cell can divide, presumably indefinitely, and gives rise to a clonal population of cells that each expresses an identical antibody molecule. The individual immortalized B cell clones, the hybridomas, are segregated and cultured separately.

The term “polyclonal antibody” refers to an antibody that is produced from a different B cell lineages within the body. A polyclonal antibody is directed to many different antigenic determinants on the target cell surface and would bind with sufficient density to allow the effector mechanisms of the immune system to work efficiently.

An immunoglobulin (antibody) structural unit are typically tetrameric glycosylated proteins. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V” or “VL” refer to the variable region of an immunoglobulin light chain, including of an Fv, scFv, dsFv or Fab.

Thus, the term antibody, as used herein, includes antibody fragments either produced by the modification of whole antibodies, or those synthesized de novo either chemically or using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301. As appreciated by one of skill in the art, various antibody fragments can be obtained by a variety of methods, for example, digestion of an intact antibody with an enzyme, such as pepsin; or de novo synthesis.

The term “aptamer” refers to oligonucleotide or peptide molecules that bind to a specific target molecule. An aptamer can include any suitable number of nucleotides. “Aptamers” refer to more than one such set of molecules. Different aptamers can have either the same or different numbers of nucleotides. Aptamers may be DNA or RNA and may be single stranded, double stranded, or contain double stranded or triple stranded regions. In embodiments, peptide aptamers consist of one (or more) short variable peptide domains, attached at both ends to a protein scaffold. Aptamers may be designed with any combination of the base modified nucleotides desired. Aptamers to a given target include nucleic acids that are identified from a candidate mixture of nucleic acids, where the aptamer is a ligand of the target, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers of the target molecule are identified. It is recognized that affinity interactions are a matter of degree; however, in this context, the “specific binding affinity” of an aptamer for its target means that the aptamer binds to its target with a much higher degree of affinity than it binds to other, non-target, components in a mixture or sample. An aptamer can be identified using any known method, including the SELEX process. See, e.g., U.S. Pat. No. 5,475,096 entitled “Nucleic Acid Ligands”. Once identified, an aptamer can be prepared or synthesized in accordance with any known method, including chemical synthetic methods and enzymatic synthetic methods.

Nucleic acid aptamers are nucleic acid species that are typically the product of engineering through repeated rounds of in vitro selection, such as SELEX (systematic evolution of ligands by exponential enrichment), to bind to various molecular targets such as small molecules, proteins, nucleic acids, and even cells, tissues and organisms. At the molecular level, aptamers bind to its target site through non-covalent interactions. Aptamers bind to these specific targets because of electrostatic interactions, hydrophobic interactions, and their +complementary shapes. In embodiments, peptide aptamers are artificial proteins selected or engineered to bind specific target molecules. These proteins may include or consist of one or more peptide loops of variable sequence displayed by a protein scaffold. They are typically isolated from combinatorial libraries and often subsequently improved by directed mutation or rounds of variable region mutagenesis and selection.

An “antigen” (Ag) is any substance that reacts specifically with antibodies or T lymphocytes (T cells). In general, antigens include molecules or portions thereof that trigger an immune response in a host (e.g., in a subject), and may be recognized by an antibody. Antigens may be foreign to a subject (e.g., as in viral or bacterial proteins, polysaccharides, or other molecules), or native to the subject (e.g., as in an autoimmune response to self-proteins, which optionally may be mutant forms of a native protein). Examples of antigens include, without limitation, viral antigens, bacterial antigens, fungal antigens, cancer or tumor antigens, and allergens. Examples of viral antigens include, but are not limited to, env, gag, rev, tar, tat, nucleocapsid proteins and reverse transcriptase from immunodeficiency viruses (e.g., HIV, FIV), such as HIV-1 gag, HIV-1 env, HIV-1 pol, HIV-1 tat, HIV-1 nef; HBV surface antigen and core antigen, HbsAG, HbcAg; HCV antigens such as hepatitis C core antigen; influenza nucleocapsid proteins; parainfluenza nucleocapsid proteins; HPV E6 and E7 such as human papilloma type 16 E6 and E7 proteins; Epstein-Barr virus LMP-1, LMP-2 and EBNA-2; herpes LAA and glycoprotein D such as HSV glycoprotein D; as well as similar proteins from other viruses. In embodiments, the biomolecule-specific binding moiety is an antibody that is reactive to a plurality of viral antigens within the same viral group. For example, a flavivirus group-reactive antibody such as the monoclonal antibody MAb 6B6C-1, dengue 4G2, or Murray Valley 4A1B-9 is reactive with arbovirus antigens within the flavivirus genus, which includes the West Nile virus, Saint Louis encephalitis virus, Japanese encephalitis virus, and dengue virus. Similarly, for example, an alphavirus group-reactive antibody such as EEE 1A4B-6 or WEE 2A2C-3 is reactive with alphavirus antigens within the alphavirus genus, which includes eastern equine encephalitis virus, western equine encephalitis virus, and Venezuelan equine encephalitis virus. Similarly, for example, a bunyavirus group-reactive antibody such as LAC 10G5.4 is reactive with bunyavirus antigens within the bunyavirus genus, which includes the California serogroup of bunyaviruses, which includes La Crosse virus. Examples of bacterial antigens include, but are not limited, to capsule antigens (e.g., protein or polysaccharide antigens such as CP5 or CP8 from the S. aureus capsule); cell wall (including outer membrane) antigens such as peptidoglycan (e.g., mucopeptides, glycopeptides, mureins, muramic acid residues, and glucose amine residues) polysaccharides, teichoic acids (e.g., ribitol teichoic acids and glycerol teichoic acids), phospholipids, hopanoids, and lipopolysaccharides (e.g., the lipid A or O-polysaccharide moieties of bacteria such as Pseudomonas aeruginosa serotype 011); plasma membrane components including phospholipids, hopanoids, and proteins; proteins and peptidoglycan found within the periplasm; fimbrae antigens, pili antigens, flagellar antigens, and S-layer antigens. S. aureus antigens can be a serotype 5 capsular antigen, a serotype 8 capsular antigen, and antigen shared by serotypes 5 and 8 capsular antigens, a serotype 336 capsular antigen, protein A, coagulase, clumping factor A, clumping factor B, a fibronectin binding protein, a fibrinogen binding protein, a collagen binding protein, an elastin binding protein, a MHC analogous protein, a polysaccharide intracellular adhesion, alpha hemolysin, beta hemolysin, delta hemolysin, gamma hemolysin, Panton-Valentine Leukocidin, exfoliative toxin A, exfoliative toxin B, V8 protease, hyaluronate lyase, lipase, staphylokinase, LukED leukocidin, an enterotoxin, toxic shock syndrome toxin-1, poly-N-succinyl beta-1→6 glucosamine, catalase, beta-lactamase, teichoic acid, peptidoglycan, a penicillin binding protein, chemotaxis inhibiting protein, complement inhibitor, Sbi, and von Willebrand factor binding protein. Non-limiting examples of fungal antigens include, but are not limited to, Candida fungal antigen components; Histoplasma fungal antigens such as heat shock protein 60 (HSP60) and other Histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components. Examples of cancer antigens include, but are not limited to, MAGE, MART-1/Melan-A, gplOO, dipeptidyl peptidase IV (DPPIV), adenosine deaminase-binding protein (ADAbp), cyclophilin b, colorectal associated antigen (CRC)-COI 7-1 A/GA733, carcinoembryonic antigen (CEA) and its immunogenic epitopes CAP-1 and CAP-2, etvβ, aml1, prostate specific antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21 ras, RCAS1, α-fetoprotein, E-cadherin, α-catenin, β-catenin, γ-catenin, p120ctn, gp100/p mel 17, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, Smad family of tumor antigens, lmp-1, P1 A, EBV-encoded nuclear antigen (EBNA)-I, brain glycogen phosphorylase, SSX-1, SSX-2 (HOM-MEL-40), SSX-1, SSX-4, SSX-5, SCP-1, CT-7, and c-erbB-2. Examples of allergens include, but are not limited to, dust, pollen, pet dander, food such as peanuts, nuts, shellfish, fish, wheat milk, eggs, soy and their derivatives, and sulphites. These lists are not meant to be limiting.

A “biomolecule-specific binding moiety” or an “analyte-specific binding moiety” is a substance that allows for selective binding to another substance (e.g. an analyte). A particular example of specific binding is that which occurs between an antibody and an antigen. Another example of specific binding is that which occurs between the prodrug described herein and the population of cells targeted by the prodrug. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. A binding agent is typically a biological or synthetic molecule that has high affinity for another molecule or macromolecule, through covalent or non-covalent bonding. Examples of a binding agent can include streptavidin, antibody, antigen, enzyme, enzyme cofactor or inhibitor, hormone, or hormone receptor. This binding agent can bind to an analyte (e.g., a protein), often in a heterogeneous population of proteins and other biologics. Thus, under designated immunoassay conditions, the specified binding agents bind to a particular analyte at least two times the background and more typically more than 10 to 100 times background.

As used herein, the terms “biomolecule” or “analyte” refer to an agent (e.g., a compound, macromolecule, or small molecule), and the like derived from a biological system (e.g., an organism, a cell, or a tissue). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, peptides, proteins, antigen binding fragments, antibodies, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). The biomolecule may facilitate target specific delivery of the payload comprised by the compound described herein. Biomolecules of particular interest may thus include proteinaceous molecules such as peptides, polypeptides, proteins or prions or any molecule which includes a protein or polypeptide component, etc., or fragments thereof. The biomolecule may be a single molecule or a complex that contains two or more molecular subunits, which may or may not be covalently bound to one another, and which may be the same or different. Thus, in addition to cells or microorganisms, such a complex biomolecule may also be a protein complex. Such a complex may thus be a homo- or hetero-multimer. Aggregates of molecules e.g., proteins may also be target analytes, for example aggregates of the same protein or different proteins. The biomolecule may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules. Examples of biomolecules conjugated to the compounds described herein to facilitate target specificity include, but are not limited to, an antibody, a biomolecule-binding portion of a receptor, a biomolecule for a receptor, an aptamer, a carbohydrate-based biomolecule, a group including galactose, a multivalent galactose, N-acetyl-galactosamine (GalNAc), multivalent GalNAc, a mannose, multivalent mannose, lactose, multivalent lactose, N-acetyl-glucosamine (GlcNAc), multivalent GlcNAc, glucose, multivalent glucose, fucose, multivalent fucose, a fatty acid, a lipoprotein, folate, thyrotropin, melanotropin, surfactant protein A, mucin, glycosylated polyaminoacids, transferrin, bisphosphonate, polyglutamate, polyaspartate, a lipophilic moiety, a cholesterol, a steroid, bile acid, vitamin B12, biotin, a fluorophore, and a peptide.

An “affimer” is a protein that binds to target proteins with affinity in the nanomolar range. It behaves similarly to an antibody by binding tightly to its target molecule. Affimers are recombinant proteins that are typically engineered to mimic molecular recognition characteristics of monoclonal antibodies.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. Nucleic acids and polynucleotides are polymers of any length, including longer lengths, e.g., 200, 300, 500, 1000, 2000, 3000, 5000, 7000, 10,000, etc. In certain embodiments the nucleic acids herein contain phosphodiester bonds. In other embodiments, nucleic acid analogs are included that may have alternate backbones, comprising, e.g., phosphoramidite, phosphorothioate, phosphorodithioate, or O-methylphosphoroamidite linkages (see, Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. A residue of a nucleic acid, as referred to herein, is a monomer of the nucleic acid (e.g., a nucleotide). The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like. A “nucleic acid moiety” as used herein is a monovalent form of a nucleic acid. In embodiments, the nucleic acid moiety is attached to the 3′ or 5′ position of a nucleotide or nucleoside.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

“Nucleotide,” as used herein, refers to a nucleoside-5′-phosphate (e.g., polyphosphate) compound, or a structural analog thereof, which can be incorporated (e.g., partially incorporated as a nucleoside-5′-monophosphate or derivative thereof) by a nucleic acid polymerase to extend a growing nucleic acid chain (such as a primer). Nucleotides may comprise bases such as adenine (A), cytosine (C), guanine (G), thymine (T), uracil (U), or analogues thereof, and may comprise 1, 2, 3, 4, 5, 6, 7, 8, or more phosphates in the phosphate group. Nucleotides may be modified at one or more of the base, sugar, or phosphate group. A nucleotide may have a label or tag attached (a “labeled nucleotide” or “tagged nucleotide”). In an embodiment, the nucleotide is a deoxyribonucleotide. In another embodiment, the nucleotide is a ribonucleotide. In embodiments, nucleotides comprise 3 phosphate groups (e.g., a triphosphate group).

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidite, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

In embodiments, “nucleotide analogue,” “nucleotide analog,” or “nucleotide derivative” shall mean an analogue of adenine (A), cytosine (C), guanine (G), thymine (T), or uracil (U) (that is, an analogue or derivative of a nucleotide comprising the base A, G, C, T or U), comprising a phosphate group, which may be recognized by DNA or RNA polymerase (whichever is applicable) and may be incorporated into a strand of DNA or RNA (whichever is appropriate). Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the —OH group at the 3′-position of deoxyribose. Nucleotide analogues and DNA polymerase-based DNA sequencing are also described in U.S. Pat. No. 6,664,079, which is incorporated herein by reference in its entirety for all purposes.

The term “nucleobase” or “base” as used herein refers to a purine or pyrimidine compound, or a derivative thereof, that may be a constituent of nucleic acid (i.e., DNA or RNA, or a derivative thereof). In embodiments, the nucleobase is a divalent purine or pyrimidine, or derivative thereof. In embodiments, the nucleobase is a monovalent purine or pyrimidine, or derivative thereof. In embodiments, the base is a derivative of a naturally occurring DNA or RNA base (e.g., a base analogue). In embodiments the base is a hybridizing base. In embodiments the base hybridizes to a complementary base. In embodiments, the base is capable of forming at least one hydrogen bond with a complementary base (e.g., adenine hydrogen bonds with thymine, adenine hydrogen bonds with uracil, guanine pairs with cytosine). Non-limiting examples of a base includes cytosine or a derivative thereof (e.g., cytosine analogue), guanine or a derivative thereof (e.g., guanine analogue), adenine or a derivative thereof (e.g., adenine analogue), thymine or a derivative thereof (e.g., thymine analogue), uracil or a derivative thereof (e.g., uracil analogue), hypoxanthine or a derivative thereof (e.g., hypoxanthine analogue), xanthine or a derivative thereof (e.g., xanthine analogue), 7-methylguanine or a derivative thereof (e.g., 7-methylguanine analogue), deaza-adenine or a derivative thereof (e.g., deaza-adenine analogue), deaza-guanine or a derivative thereof (e.g., deaza-guanine), deaza-hypoxanthine or a derivative thereof, 5,6-dihydrouracil or a derivative thereof (e.g., 5,6-dihydrouracil analogue), 5-methylcytosine or a derivative thereof (e.g., 5-methylcytosine analogue), or 5-hydroxymethylcytosine or a derivative thereof (e.g., 5-hydroxymethylcytosine analogue) moieties. In embodiments, the base is adenine, guanine, uracil, cytosine, thymine, hypoxanthine, xanthine, theobromine, caffeine, uric acid, or isoguanine, which may be optionally substituted or modified. In embodiments, the base is adenine, guanine, hypoxanthine, xanthine, theobromine, caffeine, uric acid, or isoguanine, which may be optionally substituted or modified.

The term “cleavable linker,” “cleavable tether,” or “cleavable moiety” as used herein refers to a divalent or monovalent, respectively, moiety which is capable of being separated (e.g., detached, split, disconnected, hydrolyzed, a stable bond within the moiety is broken) into distinct entities. In embodiments, a cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). In embodiments, a cleavable linker is a self-immolative linker, a trivalent linker, or a linker capable of dendritic amplification of signal, or a self-immolative dendrimer containing linker (e.g., all as described in US 2007/0009980, US 2006/0003383, and US 2009/0047699, which are incorporated by reference in their entirety for any purpose). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), hydrazine (N₂H₄)). A chemically cleavable linker is non-enzymatically cleavable. In embodiments, the cleavable linker is cleaved by contacting the cleavable linker with a cleaving agent. In embodiments, the cleaving agent is sodium dithionite (Na₂S₂O₄), weak acid, hydrazine (N₂H₄), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase (Fpg). In embodiments, the scissile linkage site includes a sequence-specific nicking site having a nucleotide sequence that is recognized and nicked by a nicking endonuclease enzyme or a uracil DNA glycosylase. The term “self-immolative” referring to a linker is used in accordance with its well understood meaning in Chemistry and Biology as used in US 2007/0009980, US 2006/0003383, and US 2009/0047699, which are incorporated by reference in their entirety for any purpose. In embodiments, “self-immolative” referring to a linker refers to a linker that is capable of additional cleavage following initial cleavage by an external stimuli. The term dendrimer is used in accordance with its well understood meaning in Chemistry. In embodiments, the term “self-immolative dendrimer” is used as described in US 2007/0009980, US 2006/0003383, and US 2009/0047699, which are incorporated by reference in their entirety for any purpose and in embodiments refers to a dendrimer that is capable of releasing all of its tail units through a self-immolative fragmentation following initial cleavage by an external stimulus.

The term “polymer” refers to a molecule including structurally unique repeating subunits (e.g., polymerized monomers). The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. For example, polymeric molecules may be based upon polyethylene glycol (PEG), tetraethylene glycol (TEG), polyvinylpyrrolidone (PVP), poly(xylene), or poly(p-xylylene). The term “polymerizable monomer” is used in accordance with its meaning in the art of polymer chemistry and refers to a compound that may covalently bind chemically to other monomer molecules (such as other polymerizable monomers that are the same or different) to form a polymer.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or

“\*MERGEFORMAT\*MERGEFORMAT —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds described herein. Thus, the compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. In embodiments, compounds may be presented with a positive charge, and it is understood an appropriate counter-ion (e.g., chloride ion, fluoride ion, or acetate ion) may also be present, though not explicitly shown. Likewise, for compounds having a negative charge (e.g.,

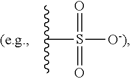

it is understood an appropriate counter-ion (e.g., a proton, sodium ion, potassium ion, or ammonium ion) may also be present, though not explicitly shown. The protonation state of the compound (e.g., a compound described herein) depends on the local environment (i.e., the pH of the environment), therefore, in embodiments, the compound may be described as having a moiety in a protonated state

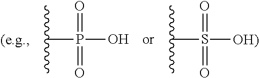

or an ionic state

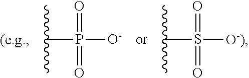

and it is understood these are interchangeable. In embodiments, the counter-ion is represented by the symbol M (e.g., M⁺ or M⁻).

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Prodrugs of the compounds described herein may be converted in vivo after administration. Additionally, prodrugs can be converted to the compounds of the present invention by chemical or biochemical methods in an ex vivo environment, such as, for example, when contacted with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist.

The terms “diagnosing” or “diagnosis” refers to the identification of a disease, condition, or injury based on symptoms or evidence presented by means that includes, but are not limited to, biopsies, imaging tests, or physical exams.

The term “monitoring” refers to observing the disease state or condition presented in a patient as a means to manage the disease or condition by methods that includes, but are not limited to, exams and biopsies.

The terms “treating” or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal) compatible with the preparation. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “prodrug” is used in accordance with its plain and ordinary meaning and refers a therapeutic agent precursor (e.g., a pharmacologically inactive molecule) that requires an enzymatic, chemical, or a combination of both enzymatic and chemical transformation in vivo to release the pharmacological active molecule or therapeutic moiety. Prodrugs contain a prodrug moiety (e.g., a chemical masking moiety) to render the therapeutic agent inactive prior to the removal of the prodrug moiety, and prodrug moieties are also often employed to improve physicochemical and pharmacokinetic properties of pharmacologically active compounds. Examples of notable prodrugs include prednisone and irinotecan, which undergo conversion in vivo to provide their active pharmacological forms, prednisolone and SN-38 (a camptothecin analog), respectively. Additional examples of prodrug include compounds as described herein, which require cleavage of the cleavable linking moiety (i.e., CLM) described herein to release the activated therapeutic moiety.

The term “drug” is used in accordance with its plain and ordinary meaning and refers to a substance which has a physiological effect (e.g., beneficial effect, is useful for treating a subject) when introduced into or to a subject (e.g., in or on the body of a subject or patient). For example, a drug can have a therapeutic effect useful for treating a condition or disease state. A drug moiety is a radical of a drug.

As used herein, the term “antibacterial drug” is used in accordance with its plain and ordinary meaning and refers to any agent capable of killing bacteria or preventing its growth. Exemplary examples of an antibacterial drug include vancomycin and cefuroxime axetil.

As used herein, the term “immunomodulator drug” is used in accordance with its plain and ordinary meaning and refers to any agent that inhibits (e.g., immunosuppressive) or accentuates (e.g., immunostimulant) the activity of immune responsive cells for the treatment of autoimmune and inflammatory disorders. Therapeutic modalities for immunomodulator drugs can include small molecules and biologics. Examples of immunomodulator drugs include, but are not limited to vaccines, monoclonal/polyclonal antibodies, pomalidomide, and prednisone.

As used herein, the term “BCS class IV drug” is used in accordance with its plain and ordinary meaning and refers to a drug defined by biopharmaceutical classification system (BCS). BCS class IV drugs may be characterized by low aqueous solubility and low intestinal permeability. Examples of BCS class IV drugs include, but are not limited to, chemotherapeutic drugs (e.g., doxorubicin, paclitaxel, and campothecin), neprilysin inhibitors (e.g., sacubitril), and anti-inflammatory drugs (e.g., mesalamine).

As used herein, the term “chemotherapeutic drug” is used in accordance with its plain and ordinary meaning and refers any agent used for the eradication of cancer cells through various mechanisms (e.g., damaging DNA, inhibiting DNA replication, interfering with proteins involved in the mitotic cycle). Types and examples of chemotherapeutic drugs include, but are not limited to, alkylating agents (e.g., cisplatin), antimetabolites (e.g., cytidine analogs such as gemicitabine, folate antagonists, purine analogs, and pyrimidine analogs), antimicrotubular agents (e.g., taxanes such as paclitaxel and topoisomerase I/II inhibitors such as irinotecan), and anti-cancer antibiotics (e.g., doxorubicin).

“Anti-cancer agent” or “anti-cancer drug” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent approved by the FDA or similar regulatory agency of a country other than the USA, for treating cancer. Examples of anti-cancer agents include, but are not limited to, anti-androgens (e.g., Casodex, Flutamide, MDV3100, or ARN-509), MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002), mTOR inhibitors, antibodies (e.g., rituxan), 5-aza-2′-deoxycytidine, doxorubicin, vincristine, etoposide, gemcitabine, imatinib (Gleevec®), geldanamycin, 17-N-Allylamino-17-Demethoxygeldanamycin (17-AAG), bortezomib, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (ICOS); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-1 receptor inhibitor; interferon agonists; interferons; interleukins; letrozole; leukemia inhibiting factor; leukocyte alpha interferon; leuprolide+estrogen+progesterone; leuprorelin; matrilysin inhibitors; matrix metalloproteinase inhibitors; MIF inhibitor; mifepristone; mismatched double stranded RNA; monoclonal antibody; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein tyrosine phosphatase inhibitors; purine nucleoside phosphorylase inhibitors; ras farnesyl protein transferase inhibitors; ras inhibitors; ras-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Gudrin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, pyrrolo benzodiazepines (e.g. tomaymycin), carboplatin, CC-1065 and CC-1065 analogs including amino-CBIs, nitrogen mustards (such as chlorambucil and melphalan), dolastatin and dolastatin analogs (including auristatins: eg. monomethyl auristatin E), anthracycline antibiotics (such as doxorubicin, daunorubicin, etc.), duocarmycins and duocarmycin analogs, enediynes (such as neocarzinostatin and calicheamicins), leptomycin derivaties, maytansinoids and maytansinoid analogs (e.g. mertansine), methotrexate, mitomycin C, taxoids, vinca alkaloids (such as vinblastine and vincristine), epothilones (e.g. epothilone B), camptothecin and its clinical analogs topotecan and irinotecan, or the like.

As used herein, the term “photosensitizer drug” is used in accordance with its plain and ordinary meaning and refers any agent that absorbs a specific wavelength of light, resulting in the generation of reactive oxygen species (ROS) from diseased cells and ultimately, cell death. Examples of photosensitizer drugs include, but are not limited to, temoporfin, radachlorin, and verteporfin.

As used herein, the term “nanoparticle” is used in accordance with its plain and ordinary meaning and refers any particle where the longest diameter of the particle is less than or equal to 1000 nanometers. Nanoparticles may be composed of any appropriate material. For example, nanoparticle cores may include appropriate metals and metal oxides thereof (e.g., a metal nanoparticle core), carbon (e.g., an organic nanoparticle core) silicon and oxides thereof (e.g., a silicon nanoparticle core) or boron and oxides thereof (e.g., a boron nanoparticle core), or mixtures thereof. Nanoparticles are used for targeted drug delivery of various payloads and differ based on their drug delivery platform, which includes lipid-based nanoparticles, inorganic nanoparticles, and polymeric nanoparticles. Examples of FDA approved nanoparticle therapeutics include, but are not limited to, Doxil® (liposomal doxorubicin), Vyxeos® (cytarabine/daunorubicin liposomal), Abraxane® (albumin-bound paclitaxel), and Venofer® (iron sucrose).

The term “disease” refer to a state of being or health status of a patient or subject capable of being treated with the compounds or methods provided herein, and the anatomical sites affected by disease is referred to as “pathological entity” or “pathological site.” Examples of diseases, disorders, or conditions include, but are not limited to cancer. Additional examples of diseases and conditions to be treated with compounds or methods provided herein include adenocarcinomas, ALS, bacterial infections, benign tumors, diseases affecting bone and/or bone marrow, brain cancer, breast tumors, diseases affecting the cerebellum, carcinomas, chronic inflammation, chronic wounds, dopaminergic dysfunction, gastric cancer and gastrointestinal diseases, diseases affecting gastric glands, glioma, graft-versus-host disease, head injury, hepatic tumors, Hodgkin's lymphoma, hypoxia and ischemia, inaccessible hypoxic tumor regions, infections, inflamed central nervous system, Kaposi's sarcoma, leukemias, liver cancers, lung tumors, lymphoid cancers, lymphomas, malignant tumors, medulloblastoma, melanoma, metastatic tumors, microsatellite tumors, multiple sclerosis, multiple myeloma, muscular dystrophy, myocardial infarction, osteogenesis imperfect, ovarian cancers, primary tumors, prostate tumors, radiation damages, sarcomas, solid tumors, spinal chord dysfunction, diseases affecting spleen, stroke, subarachnoid space for autoimmune diseases, subcutaneous wounds, diseases affecting the thymus, and tissue damage. Examples of pathological entities or pathological sites capable of being treated with the compounds or methods provided herein include kidney, breast, lung, bladder, colon, ovarian, prostate, pancreas, stomach, brain, head and neck, skin, uterine, testicular, esophagus, and liver.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” and “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refers to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

The term “tropic cell” is used in accordance with its plain and ordinary meaning and refers any cell with a migratory affinity for specific organs. An exemplary example includes but is not limited to, brain-tropic breast cancer cells, which are breast cancer cells with the ability to develop brain metastases.

The term “thiolation reagent” is used in accordance with its plain ordinary meaning in the arts and refers to a substance (e.g., a compound or solution) which participates in chemical reaction and results in the formation of a thio containing moiety (e.g., —SS— or

“\*MERGEFORMAT\*MERGEFORMAT —SSCH₃) (e.g., between bioconjugate reactive moieties, between a bioconjugate reactive moiety and the thiolation reagent) in aqueous solution. In embodiments, the thiolation reagent is capable of converting —SH to —SSMe in water. In embodiments, the thiolation reagent is

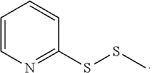

In embodiments the thiolation reagent is

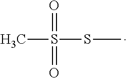

In embodiments, the thiolation reagent is

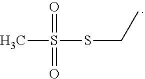

In embodiments, the thiolation reagent is a compound (e.g., a reagent) described in Mandal and Basu (RSC Adv., 2014, 4, 13854) or Musiejuk and Witt (Organic Preparations and Procedures International, 47:95-131, 2015), which are incorporated by reference in their entirety for all purposes. Non-limiting examples of a thiolation reagent include 1,2-propanediol, 3-(2-pyridinyldithio)-; 1,4,7,10-tetraazacyclododecane, 1-[2-(2-pyridinyldithio)ethyl]-; 1,4,7,10-tetraazacyclododecane-1,4,7-triacetic acid, α1,α4,α7,2,5,8,11-heptamethyl-10-[(is)-1-methyl-2-oxo-2-[[2-(2-pyridinyldithio)ethyl]amino]ethyl]-, (α1s,α4s,α7s,2s,5s,8s,11s)-; 1-butanol, 4-(2-pyridinyldithio)-; 1h,7h-pyrazolo[1,2-a]pyrazole-1,7-dione, 2,3,6-trimethyl-5-[(2-pyridinyldithio)methyl]-; 1-hexanol, 6-(2-pyridinyldithio)-; 1h-thieno[3,4-d]imidazole-4-pentanamide, hexahydro-2-oxo-n-[2-(2-pyridinyldithio)ethyl]-, (4s)-; 1h-thieno[3,4-d]imidazole-4-pentanamide, hexahydro-2-oxo-n-[2-(2-pyridinyldithio)ethyl]-, [3as-(3aα,4β,6aα)]-(9ci); 1h-thieno[3,4-d]imidazole-4-pentanamide, hexahydro-2-oxo-n-[6-[[1-oxo-3-(2-pyridinyldithio)propyl]amino]hexyl]-, (3as,4s,6ar)-; 1h-thieno[3,4-d]imidazole-4-pentanamide, hexahydro-2-oxo-n-[6-[[1-oxo-3-(2-pyridinyldithio)propyl]amino]hexyl]-, (4s)-; 1-propanamine, 2-methyl-2-(2-pyridinyldithio)-; 1-propanamine, 3-(2-pyridinyldithio)-; 1-propanol, 3-(2-pyridinyldithio)-; 2(1h)-pyridinone, 6-(2-pyridinyldithio)-; 2-buten-1-ol, 3-methyl-4-(2-pyridinyldithio)-, (2e)-; 2-naphthalenol, 6-(2-pyridinyldithio)-; 2-propenamide, n-[2-(2-pyridinyldithio)ethyl]-; 2-pyridinamine, 6-(2-pyridinyldithio)-; 2-pyridinecarbonitrile, 6-(2-pyridinyldithio)-; 3,6,9,12,15-pentaoxaheptadecan-1-amine, 17-(2-pyridinyldithio)-; 3-buten-1-ol, 2-(2-pyridinyldithio)-; 3-pyridinamine, 2-(2-pyridinyldithio)-; 3-pyridinamine, 6-(2-pyridinyldithio)-; 3-pyridinecarbonitrile, 2-(2-pyridinyldithio)-; 3-pyridinecarbonitrile, 6-(2-pyridinyldithio)-; 3-pyridinol, 2-(2-pyridinyldithio)-; 3-pyridinol, 6-(2-pyridinyldithio)-; 4,7,10,13,16,19-hexaoxa-22-azapentacosanoic acid, 23-oxo-25-(2-pyridinyldithio)-, 1,1-dimethylethyl ester; 4,7,10,13-tetraoxa-16-azanonadecanoic acid, 17-oxo-19-(2-pyridinyldithio)-, 2,5-dioxo-1-pyrrolidinyl ester; 4,7,10,13-tetraoxapentadecanoic acid, 15-(2-pyridinyldithio)-, 2,5-dioxo-1-pyrrolidinyl ester; 4-pyridinamine, 2-(2-pyridinyldithio)-; 4-pyridinecarbonitrile, 2-(2-pyridinyldithio)-; 4-pyridinol, 2-(2-pyridinyldithio)-; acetamide, 2-(aminooxy)-n-[2-(2-pyridinyldithio)ethyl]-; acetamide, 2-amino-n-[2-(2-pyridinyldithio)ethyl]-; acetamide, 2-chloro-n-[2-(2-pyridinyldithio)ethyl]-; acetamide, n-[(2-pyridinyldithio)methyl]-; acetamide, n-[2-(2-pyridinyldithio)ethyl]-; acetic acid, 2-[[16-oxo-18-(2-pyridinyldithio)-3,6,9,12-tetraoxa-15-azaoctadec-1-yl]oxy]-, 2,5-dioxo-1-pyrrolidinyl ester; adenosine, 3′-deoxy-3′-(2-pyridinyldithio)-(9ci); benzamide, 4-hydrazinyl-n-[2-(2-pyridinyldithio)ethyl]-; benzenamine, 2-(2-pyridinyldithio)-; benzenamine, 2,4-dinitro-n-[2-(2-pyridinyldithio)ethyl]-; benzenemethanol, 2-(2-pyridinyldithio)-; benzenemethanol, 3-(2-pyridinyldithio)-; benzenemethanol, 4-(2-pyridinyldithio)-; benzenepropanamide, α-(acetylamino)-n-[2-(2-pyridinyldithio)ethyl]-, (as)-; benzenesulfonamide, 4-methyl-n-[2-(2-pyridinyldithio)ethyl]-; benzoic acid, 2-(2-pyridinyldithio)-, 1,1′-(3-oxospiro[isobenzofuran-1(3h),9′-[9h]xanthene]-3′,6′-diyl) ester; benzoic acid, 2-(2-pyridinyldithio)-, 6′-methoxy-3-oxospiro[isobenzofuran-1(3h),9′-[9h]xanthen]-3′-yl ester; benzoic acid, 2-[[3,5-dichloro-4-[[14-(2-pyridinyldithio)-3,6,9,12-tetraoxatetradec-1-yl]oxy]phenyl]amino]-; benzoic acid, 2-[[3,5-dichloro-4-[[17-(2-pyridinyldithio)-3,6,9,12,15-pentaoxaheptadec-1-yl]oxy]phenyl]amino]-; benzoic acid, 2-[[3,5-dichloro-4-[2-(2-pyridinyldithio)ethoxy]phenyl]amino]-; benzoic acid, 2-[[3,5-dichloro-4-[2-[2-(2-pyridinyldithio)ethoxy]ethoxy]phenyl]amino]-; benzoic acid, 2-[[3,5-dichloro-4-[2-[2-[2-(2-pyridinyldithio)ethoxy]ethoxy]ethoxy]phenyl]amino]-; benzoic acid, 2-[[3,5-dichloro-4-[2-[2-[2-[2-(2-pyridinyldithio)ethoxy]ethoxy]ethoxy]ethoxy]phenyl]amino]-; benzoic acid, 4-[(2-pyridinyldithio)methyl]-; benzoic acid, 4-[(2-pyridinyldithio)methyl]-, 2,5-dioxo-1-pyrrolidinyl ester; benzoic acid, 4-[1-(2-pyridinyldithio)ethyl]-, 2,5-dioxo-1-pyrrolidinyl ester; benzothiazole, 2-(2-pyridinyldithio)-; butanamide, 2-(acetylamino)-4-(2-pyridinyldithio)-, (s)-(9ci); butanamide, 2-[(1-oxo-2-propenyl)amino]-4-(2-pyridinyldithio)-, (s)-(9ci); butanimidamide, 4-(2-pyridinyldithio)-; butanoic acid, 2-(dimethylamino)-4-(2-pyridinyldithio)-; butanoic acid, 2-(dimethylamino)-4-(2-pyridinyldithio)-, 2,5-dioxo-1-pyrrolidinyl ester; butanoic acid, 3-(2-pyridinyldithio)-, (r)-(9ci); butanoic acid, 3-(2-pyridinyldithio)-, 2,5-dioxo-1-pyrrolidinyl ester; butanoic acid, 3-methyl-3-(2-pyridinyldithio)-; butanoic acid, 3-methyl-3-(2-pyridinyldithio)-, 2,5-dioxo-1-pyrrolidinyl ester; butanoic acid, 3-methyl-3-(2-pyridinyldithio)-, hydrazide; butanoic acid, 4-(2-pyridinyldithio)-; butanoic acid, 4-(2-pyridinyldithio)-, 2,3,4,5,6-pentafluorophenyl ester; butanoic acid, 4-(2-pyridinyldithio)-, 2,5-dioxo-1-pyrrolidinyl ester; butanoic acid, 4-(2-pyridinyldithio)-, 2,5-dioxo-3-sulfo-1-pyrrolidinyl ester; butanoic acid, 4-(2-pyridinyldithio)-, hydrazide; butanoic acid, 4-(2-pyridinyldithio)-2-sulfo-; butanoic acid, 4-(2-pyridinyldithio)-2-sulfo-, 1-(2,5-dioxo-1-pyrrolidinyl) ester; butanoic acid, 4-[[1-oxo-3-(2-pyridinyldithio)propyl]amino]-, 2,5-dioxo-1-pyrrolidinyl ester; butanoic acid, 4-[2-[2-(2-pyridinyldithio)ethoxy]ethoxy]-, 2,5-dioxo-1-pyrrolidinyl ester; butanoic acid, 4-oxo-4-[[4-(2-pyridinyldithio)phenyl]amino]-, hydrazide; carbamic acid, [2-(2-pyridinyldithio)ethyl]-, 1,1-dimethylethyl ester (9ci); carbamic acid, [2-oxo-2-[[2-(2-pyridinyldithio)ethyl]amino]ethoxy]-, 1,1-dimethylethyl ester (9ci); carbamic acid, [3-(methylthio)-1-[(2-pyridinyldithio)methyl]propyl]-, 1,1-dimethylethyl ester, (s)-(9ci); carbamic acid, n-methyl-n-[2-(2-pyridinyldithio)ethyl]-, 1,1-dimethylethyl ester; carbonic acid, 1h-benzotriazol-1-yl 2-(2-pyridinyldithio)ethyl ester; carbonic acid, 2-methyl-2-(2-pyridinyldithio)propyl 4-nitrophenyl ester; carbonic acid, 4-nitrophenyl 2-(2-pyridinyldithio)ethyl ester; carbonic acid, 4-nitrophenyl 2-(2-pyridinyldithio)propyl ester; carbonochloridic acid, 2-(2-pyridinyldithio)ethyl ester; carbonochloridic acid, 3-(2-pyridinyldithio)propyl ester; cas index name; cytidine, n-[1-oxo-3-(2-pyridinyldithio)propyl]-(9ci); d-phenylalanine, n-acetyl-, 2-(2-pyridinyldithio)ethyl ester; ethanamine, 2-(2-pyridinyldithio)-; ethanamine, n,n-dimethyl-2-(2-pyridinyldithio)-; ethanol, 2-(2-pyridinyldithio)-; ethanol, 2-[2-(2-pyridinyldithio)ethoxy]-; ethanol, 2-[2-[2-[2-(2-pyridinyldithio)ethoxy]ethoxy]ethoxy]-; glycine, n-[2-[bis(carboxymethyl)amino]ethyl]-n-[2-oxo-2-[[2-(2-pyridinyldithio)ethyl]amino]ethyl]-; guanosine, 3′-deoxy-3′-(2-pyridinyldithio)-(9ci); heptanoic acid, 7-[[1-oxo-3-(2-pyridinyldithio)propyl]amino]-, 2,5-dioxo-1-pyrrolidinyl ester; hexadecanoic acid, 16-(2-pyridinyldithio)-; hexanamide, 2,6-diamino-n-[2-(2-pyridinyldithio)ethyl]-, (2s)-; hexanamide, n-(2-ethoxy-1,3-dioxan-5-yl)-6-(2-pyridinyldithio)-; hexanamide, n-(cis-2-methoxy-1,3-dioxan-5-yl)-6-(2-pyridinyldithio)-; hexanamide, n-(trans-2-methoxy-1,3-dioxan-5-yl)-6-(2-pyridinyldithio)-; hexanamide, n-[2-(2-propyn-1-yloxy)-1,1-bis[(2-propyn-1-yloxy)methyl]ethyl]-6-(2-pyridinyldithio)-; hexanamide, n-[2-hydroxy-1-(hydroxymethyl)ethyl]-6-(2-pyridinyldithio)-; hexanoic acid, 6-(2-pyridinyldithio)-; hexanoic acid, 6-(2-pyridinyldithio)-, 2,5-dioxo-1-pyrrolidinyl ester; hexanoic acid, 6-[[1-oxo-3-(2-pyridinyldithio)propyl]amino]-; hexanoic acid, 6-[[1-oxo-3-(2-pyridinyldithio)propyl]amino]-, 2,5-dioxo-1-pyrrolidinyl ester; hexanoic acid, 6-[[1-oxo-3-(2-pyridinyldithio)propyl]amino]-, 2,5-dioxo-3-sulfo-1-pyrrolidinyl ester; hydrazinecarboxylic acid, 2-(2-pyridinyldithio)ethyl ester; hydrazinecarboxylic acid, 2-[1-oxo-3-(2-pyridinyldithio)propyl]-, 1,1-dimethylethyl ester; index name not yet assigned; 1-alanine, 3-(2-pyridinyldithio)-; 1-ornithinamide, l-valyl-n5-(aminocarbonyl)-n-[4-[[[[[2-(2-pyridinyldithio)ethyl]amino]carbonyl]oxy]methyl]phenyl]-; octanoic acid, 6,8-bis(2-pyridinyldithio)-; pentanamide, 2-amino-4-methyl-n-[2-(2-pyridinyldithio)ethyl]-, (2s)-; pentanediamide, 2-amino-n1-[2-(2-pyridinyldithio)ethyl]-, (2s)-; pentanoic acid, 4-(2-pyridinyldithio)-; pentanoic acid, 4-(2-pyridinyldithio)-, (4s)-; pentanoic acid, 4-(2-pyridinyldithio)-, 2,3,4,5,6-pentafluorophenyl ester; pentanoic acid, 4-(2-pyridinyldithio)-, 2,5-dioxo-1-pyrrolidinyl ester; pentanoic acid, 4-(2-pyridinyldithio)-, 2,5-dioxo-1-pyrrolidinyl ester, (4s)-; pentanoic acid, 4-(2-pyridinyldithio)-, 2,5-dioxo-3-sulfo-1-pyrrolidinyl ester; pentanoic acid, 4-methyl-4-(2-pyridinyldithio)-; pentanoic acid, 4-methyl-4-(2-pyridinyldithio)-, 2,3,4,5,6-pentafluorophenyl ester; pentanoic acid, 4-methyl-4-(2-pyridinyldithio)-, 2,5-dioxo-1-pyrrolidinyl ester; pentanoic acid, 4-methyl-4-(2-pyridinyldithio)-, 2,5-dioxo-3-sulfo-1-pyrrolidinyl ester; pentanoic acid, 5-(2-pyridinyldithio)-; pentanoic acid, 5-(2-pyridinyldithio)-, 2,5-dioxo-1-pyrrolidinyl ester; phenol, 2-(2-pyridinyldithio)-; propanamide, 3-(2-pyridinyldithio)-; propanamide, 3-(2-pyridinyldithio)-n-[(3,3a,7,7a-tetrahydro-1,3-dioxo-4,7-epoxyisobenzofuran-4(1h)-yl)methyl]-; propanamide, n-(2-aminoethyl)-3-(2-pyridinyldithio)-; propanamide, n-(2-hydroxyethyl)-3-(2-pyridinyldithio)-; propanamide, n-(3-aminopropyl)-3-(2-pyridinyldithio)-; propanamide, n-(6-aminohexyl)-3-(2-pyridinyldithio)-; propanamide, n,n′-1,4-butanediylbis[3-(2-pyridinyldithio)-; propanamide, n,n′-1,6-hexanediylbis[3-(2-pyridinyldithio)-; propanamide, n-[[(3as,4r,7s,7ar)-1,3,3a,4,7,7a-hexahydro-1,3-dioxo-4,7-epoxyisobenzofuran-4-yl]methyl]-3-(2-pyridinyldithio)-; propanamide, n-octadecyl-3-(2-pyridinyldithio)-; propanenitrile, 3-(2-pyridinyldithio)-; propanoic acid, 2-bromo-2-methyl-, 2-(2-pyridinyldithio)ethyl ester; propanoic acid, 2-bromo-2-methyl-, 3-(2-pyridinyldithio)propyl ester; propanoic acid, 3-(2-pyridinyldithio)-; propanoic acid, 3-(2-pyridinyldithio)-, 2,3,4,5,6-pentafluorophenyl ester; propanoic acid, 3-(2-pyridinyldithio)-, 2,5-dihydro-2,5-dioxo-1h-pyrrol-1-yl ester; propanoic acid, 3-(2-pyridinyldithio)-, 2,5-dioxo-1-pyrrolidinyl ester; propanoic acid, 3-(2-pyridinyldithio)-, 2,5-dioxo-3-sulfo-1-pyrrolidinyl ester; propanoic acid, 3-(2-pyridinyldithio)-, hydrazide; propanoic acid, 3-(2-pyridinyldithio)-, methyl ester; propanoic acid, 3-[[13-oxo-15-(2-pyridinyldithio)-3,6,9-trioxa-12-azapentadec-1-yl]oxy]-; propanoic acid, 3-[[19-oxo-21-(2-pyridinyldithio)-3,6,9,12,15-pentaoxa-18-azaheneicos-1-yl]oxy]-; propanoic acid, 3-[[19-oxo-21-(2-pyridinyldithio)-3,6,9,12,15-pentaoxa-18-azaheneicos-1-yl]oxy]-, 2,5-dioxo-1-pyrrolidinyl ester; propanoic acid, 3-[[25-oxo-27-(2-pyridinyldithio)-3,6,9,12,15,18,21-heptaoxa-24-azaheptacos-1-yl]oxy]-; propanoic acid, 3-[[25-oxo-27-(2-pyridinyldithio)-3,6,9,12,15,18,21-heptaoxa-24-azaheptacos-1-yl]oxy]-, 2,5-dioxo-1-pyrrolidinyl ester; propanoic acid, 3-[2-[2-(2-pyridinyldithio)ethoxy]ethoxy]-, 1,1-dimethylethyl ester; propanoic acid, 3-[2-[2-[2-(2-pyridinyldithio)ethoxy]ethoxy]ethoxy]-; propanoic acid, 3-[2-[2-[2-[[1-oxo-3-(2-pyridinyldithio)propyl]amino]ethoxy]ethoxy]ethoxy]-; propanoic acid, 3-[2-[2-[2-[[1-oxo-3-(2-pyridinyldithio)propyl]amino]ethoxy]ethoxy]ethoxy]-, 2,5-dioxo-1-pyrrolidinyl ester; pyridine, 2-(2-naphthalenyldithio)-; pyridine, 2-(2-propen-1-yldithio)-; pyridine, 2-(3,6,9,12-tetraoxapentadec-14-yn-1-yldithio)-; pyridine, 2-(butyldithio)-; pyridine, 2-(cyclohexyldithio)-; pyridine, 2-(ethyldithio)-; pyridine, 2-(hexyldithio)-; pyridine, 2-(methyldithio)-; pyridine, 2-(phenyldithio)-; pyridine, 2-(propyldithio)-; pyridine, 2,2′-[1,2-ethanediylbis(dithio)]bis-(9ci); pyridine, 2,2′-[1,2-ethanediylbis(oxy-2,1-ethanediyldithio)]bis-; pyridine, 2,2′-dithiobis-; pyridine, 2-[(1,1-diethylpropyl)dithio]-; pyridine, 2-[(1,1-dimethylethyl)dithio]-; pyridine, 2-[(1-methylethyl)dithio]-; pyridine, 2-[(1-methylpropyl)dithio]-; pyridine, 2-[(2,4,6-trimethylphenyl)dithio]-; pyridine, 2-[(2,4-dinitrophenyl)dithio]-; pyridine, 2-[(2-isocyanatoethyl)dithio]-; pyridine, 2-[(2-nitrophenyl)dithio]-; pyridine, 2-[(3p)-cholest-5-en-3-yldithio]-; pyridine, 2-[(4-chlorophenyl)dithio]-; pyridine, 2-[(4-methylphenyl)dithio]-; pyridine, 2-[(4-nitrophenyl)dithio]-; pyridine, 2-[(phenylmethyl)dithio]-; pyridine, 2-[[2-(tetrahydro-2h-thiopyran-2-yl)ethyl]dithio]-; pyridine, 2-[[2-[2-(2-methoxyethoxy)ethoxy]ethyl]dithio]-; pyridine, 2-[[3-(trimethoxysilyl)propyl]dithio]-; pyridine, 2-bromo-6-(2-pyridinyldithio)-; pyridine, 3-bromo-2-(2-pyridinyldithio)-; pyridine, 4-bromo-2-(2-pyridinyldithio)-; pyridine, 4-chloro-2-(2-pyridinyldithio)-; pyridine, 5-bromo-2-(2-pyridinyldithio)-; thymidine 5′-(tetrahydrogen triphosphate), 3′-deoxy-3′-[[1-oxo-3-(2-pyridinyldithio)propyl]amino]-(9ci); uridine 5′-(tetrahydrogen triphosphate), 2′-deoxy-5-[2-[[1-oxo-3-(2-pyridinyldithio)propyl]amino]ethenyl]-; uridine 5′-(tetrahydrogen triphosphate), 2′-deoxy-5-[2-oxo-2-[[6-[[1-oxo-3-(2-pyridinyldithio)propyl]amino]hexyl]amino]ethyl]-(9ci); uridine 5′-(tetrahydrogen triphosphate), 2′-deoxy-5-[3-[[1-oxo-3-(2-pyridinyldithio)propyl]amino]-1-propen-1-yl]-; uridine, 2′-amino-2′,3′-dideoxy-3′-(2-pyridinyldithio)-(9ci); uridine, 2′-deoxy-5-[3-oxo-3-[[2-[[1-oxo-3-(2-pyridinyldithio)propyl]amino]ethyl]amino]propyl]-(9ci); uridine, 5′-o-[bis(4-methoxyphenyl)phenylmethyl]-2′-deoxy-5-[3-oxo-3-[[2-[[1-oxo-3-(2-pyridinyldithio)propyl]amino]ethyl]amino]propyl]-(9ci); or β-alanine, n-[1-oxo-3-(2-pyridinyldithio)propyl]-2,5-dioxo-1-pyrrolidinyl ester.

### II. Compositions & Kits

In an aspect is provided a prodrug having the formula

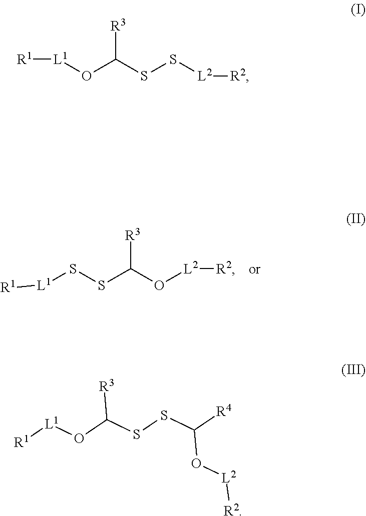

R¹ is a first drug moiety. R² is a second drug moiety, protein moiety, nucleic acid moiety, a polymer moiety, nanoparticle, detectable agent, or a tropic cell. R³ and R⁴ are independently hydrogen, halogen, —CCl₃, —CBr₃, —CF₃, —CI₃,

“\*MERGEFORMAT\*MERGEFORMAT —CHCl₂, —CHBr₂, —CHF₂, —CHI₂, —CH₂Cl, —CH₂Br, —CH₂F, —CH₂I, —CN, —OH, —NH₂, —COOH,

“\*MERGEFORMAT\*MERGEFORMAT —CONH₂, —NO₂, —SH, —SO₃H, —SO₄H, —SO₂NH₂, —NHNH₂, —ONH₂, —NHC(O)NHNH₂,

“\*MERGEFORMAT\*MERGEFORMAT —NHC(O)NH₂, —NHSO₂H, —NHC(O)H, —NHC(O)OH, —NHOH, —OCCl₃, —OCF₃, —OCBr₃, —OCI₃,

“\*MERGEFORMAT\*MERGEFORMAT —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl groups. ^{L} and L² are independently covalent linkers.

In embodiments, prodrugs readily undergo chemical changes under physiological conditions to provide a therapeutic compound (i.e., a drug). Prodrugs described herein may be converted in vivo after administration. Additionally, in embodiments, prodrugs can be converted to compounds by chemical or biochemical methods in an ex vivo environment, such as, for example, when contacted with a suitable enzyme or chemical reagent.

In embodiments, R¹ is a drug moiety (e.g., a monovalent compound described herein). In embodiments, R¹ is an antibacterial drug moiety. In embodiments, R¹ is an immunomodulator drug moiety. In embodiments, R¹ is a photosensitizer drug moiety. In embodiments, R¹ is a chemotherapeutic drug moiety.

In embodiments, R¹ is a BCS class IV drug moiety (e.g., a BCS class IV drug). In embodiments, R¹ is a drug moiety characterized by low aqueous solubility and low intestinal permeability.

In embodiments, R¹ is a temozolomide moiety. In embodiments, R¹ is a carboplatin moiety. In embodiments, R¹ is a cyclophosphamide moiety. In embodiments, R¹ is a docetaxel moiety. In embodiments, R¹ is a doxorubicin moiety. In embodiments, R¹ is a gemcitabine moiety. In embodiments, R¹ is a methotrexate moiety. In embodiments, R¹ is a paclitaxel moiety. In embodiments, R¹ is a sunitinib moiety. In embodiments, R¹ is a cisplatin moiety. In embodiments, R¹ is a 5-fluorouracil moiety. In embodiments, R¹ is a 7-ethyl-10-hydroxycamptothecin (SN-38) moiety.

In embodiments, R¹ is a DNA alkylator. In embodiments, R¹ is a DNA alkylator capable of binding to the minor groove of DNA. An example of a DNA alkylator capable of binding to the minor groove of DNA is duocarmycin. Disulfide-containing prodrugs of duocarmycins exploit the in situ formation of an activated cyclopropane following the bioreduction of the disulfide moiety, which facilitates DNA alkylation via a reaction between the activated cyclopropane of duocarmycins with a nucleobase of DNA as described by Felber, J. G. et al. (ACS Cent Sci. 2023 Mar. 28; 9(4):763-776). In embodiments, R¹ is a duocarmycin molecule or an analogue thereof. In embodiments, the prodrug has the formula:

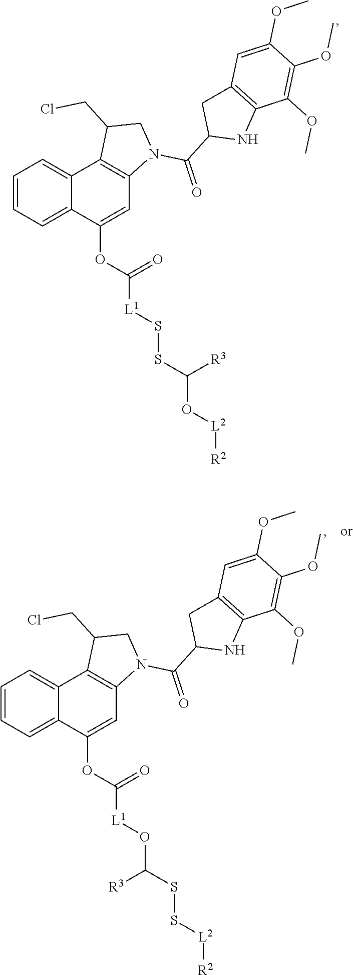

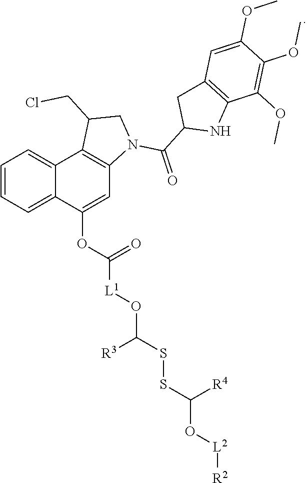

In embodiments, the prodrug has the formula:

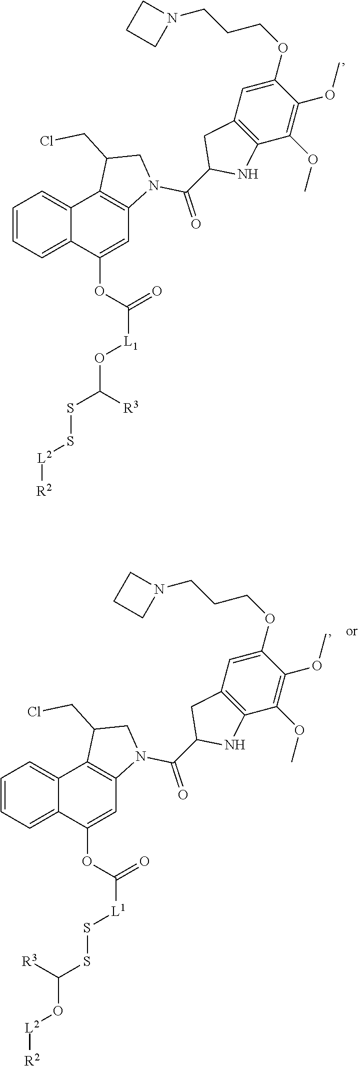

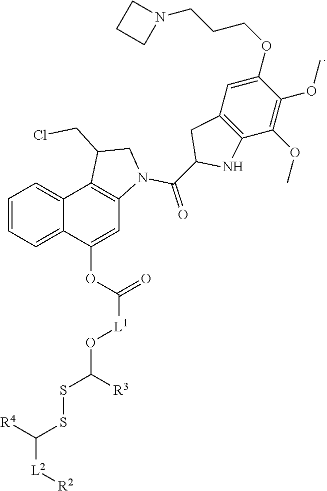

In embodiments, R¹ is a monovalent doxorubicin (DOX), monovalent paclitaxel (PTX), monovalent camptothecin (CPT), monovalent gemcitabine (GEM), monovalent AZD8055 (i.e., having the structure

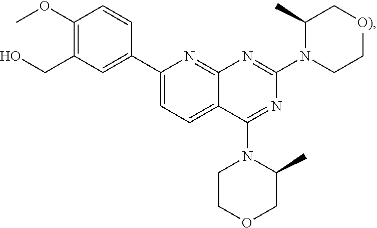

or a derivative thereof.

In embodiments, R¹ is a monovalent doxorubicin (DOX), where the compound described herein is attached to the monovalent doxorubicin moiety or derivative thereof at the amine of the daunosamine sugar of the doxorubicin (DOX) moiety as described in Pereira et al. Mol Pharm. 2019 Apr. 1; 16(4):1573-1585. In embodiments, R¹ is a monovalent paclitaxel (PTX), where the compound described herein is attached to the monovalent paclitaxel moiety or derivative thereof at 2′ position as described in Skwarczynski et al. J Med Chem. 2006 Dec. 14; 49(25):7253-69. In embodiments, R¹ is a monovalent camptothecin (CPT), where the compound described herein is attached to the monovalent camptothecin moiety or derivative thereof at C20 position as described in Checa-Chavarria et al. Mol Pharm. 2021 Apr. 5; 18(4): 1558-1572. In embodiments, R¹ is a monovalent gemcitabine (GEM), where the compound described herein is attached to the monovalent gemcitabine moiety or derivative thereof at 5′ position of the deoxycytidine or at C4 nitrogen of the cytosine as described in Pandit et al. Genes (Basel). 2022 March; 13(3): 466.

In embodiments, R² is a second drug moiety. In embodiments, R² is a protein moiety. In embodiments, R² is a nucleic acid moiety. In embodiments, R² is a polymer moiety. In embodiments, R² is a nanoparticle. In embodiments, R² is a detectable agent moiety.

In embodiments, R² is a second drug moiety. In embodiments, R² is an antibacterial drug moiety. In embodiments, R² is an immunomodulator drug moiety. In embodiments, R² is a photosensitizer drug moiety. In embodiments, R² is a chemotherapeutic drug moiety.

In embodiments, R² is a temozolomide moiety. In embodiments, R² is a carboplatin moiety. In embodiments, R² is a cyclophosphamide moiety. In embodiments, R² is a docetaxel moiety. In embodiments, R² is a doxorubicin moiety. In embodiments, R² is a gemcitabine moiety. In embodiments, R² is a methotrexate moiety. In embodiments, R² is a paclitaxel moiety. In embodiments, R² is a sunitinib moiety. In embodiments, R² is a cisplatin moiety. In embodiments, R² is a 5-fluorouracil moiety. In embodiments, R² is a 7-ethyl-10-hydroxycamptothecin (SN-38) moiety.

In embodiments, R² is a DNA alkylator. In embodiments, R² is a DNA alkylator capable of binding to the minor groove of DNA. An example of a DNA alkylator capable of binding to the minor groove of DNA is duocarmycin. Disulfide-containing prodrugs of duocarmycins exploit the in situ formation of an activated cyclopropane following the bioreduction of the disulfide moiety, which facilitates DNA alkylation via a reaction between the activated cyclopropane of duocarmycins with a nucleobase of DNA as described by Felber, J. G. et al. (ACS Cent Sci. 2023 Mar. 28; 9(4):763-776). In embodiments, R² is a duocarmycin molecule or an analogue thereof and includes the embodiments of R¹.

In embodiments, R² is a monovalent doxorubicin (DOX), monovalent paclitaxel (PTX), monovalent camptothecin (CPT), monovalent gemcitabine (GEM), monovalent AZD8055 (i.e., having the structure

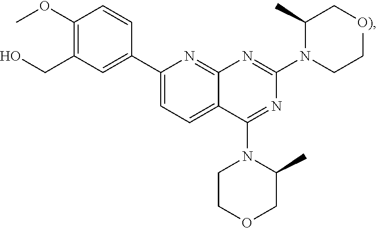

or a derivative thereof.

In embodiments, R² is a monovalent doxorubicin (DOX), where the compound described herein is attached to the monovalent doxorubicin moiety or derivative thereof at the amine of the daunosamine sugar of the doxorubicin (DOX) moiety as described in Pereira et al. Mol Pharm. 2019 Apr. 1; 16(4):1573-1585. In embodiments, R² is a monovalent paclitaxel (PTX), where the compound described herein is attached to the monovalent paclitaxel moiety or derivative thereof at 2′ position as described in Skwarczynski et al. J Med Chem. 2006 Dec. 14; 49(25):7253-69. In embodiments, R² is a monovalent camptothecin (CPT), where the compound described herein is attached to the monovalent camptothecin moiety or derivative thereof at C20 position as described in Checa-Chavarria et al. Mol Pharm. 2021 Apr. 5; 18(4): 1558-1572. In embodiments, R² is a monovalent gemcitabine (GEM), where the compound described herein is attached to the monovalent gemcitabine moiety or derivative thereof at 5′ position of the deoxycytidine or at C4 nitrogen of the cytosine as described in Pandit et al. Genes (Basel). 2022 March; 13(3): 466.

In embodiments, R² is a protein moiety. In embodiments, the protein moiety is an antibody or antigen-binding antibody fragment. In embodiments, R² is a protein, wherein the protein is an antibody or antigen-binding antibody fragment. In embodiments, R² is IgG1 antibody, IgG2a antibody, IgG3 antibody, IgG4 antibody, Fab, F(ab′)₂, scFv, or VHH. In embodiments, R² is IgG1 antibody. In embodiments, R² is an IgG2 antibody. In embodiments, R² is an IgG2a antibody. In embodiments, R² is an IgG3 antibody. In embodiments, R² is an IgG4 antibody. In embodiments, R² is a Fab. In embodiments, R² is a F(ab′)₂. In embodiments, R² is a scFv. In embodiments, R² is or a VHH.

In embodiments, R² is an antibody or antigen-binding antibody fragment capable of specifically binding to c-Met, 5T4, ADAM9, AG7, AXL, B7-H3, BCMA, CCR7, CD123, CD166, CD19, CD20, CD205, CD22, CD228, CD25, CD276, CD30, CD33, CD37, CD38, CD46, CD56, CD71, CD74, CD79, CDH6, CEACAM5, CEACAM6, EGFR, ENPP3, folate receptor alpha, Globo H, gpNMB, GPR20, HER2, HER3, HLA-DR, IGF-1, IGFR, integrin beta-6, LIV-1, mesothelin, MUC1, NaPi2b, nectin-4, PSMA, ROR1, STn, TF, tissue factor, and Trop-2.

In embodiments, R² is an antibody or antigen-binding antibody fragment capable of specifically binding to Trop-2, CEACAM5, CEACAM6, CD20, CD22, CD30, CD46, CD74, HER2, folate receptor, or HLA-DR.

In embodiments, R² is nucleic acid moiety. In embodiments, R² includes a nucleobase and a 5-membered ring sugar (e.g., either ribose or deoxyribose). In embodiments, R² includes a nucleobase that is a cytosine or a derivative thereof. In embodiments, R² includes a nucleobase that is guanine or a derivative thereof. In embodiments, R² includes a nucleobase that is adenine or a derivative thereof. In embodiments, R² includes a nucleobase that is thymine or a derivative thereof. In embodiments, R² includes a nucleobase that is uracil or a derivative thereof. In embodiments, R² includes a nucleobase that is hypoxanthine or a derivative thereof. In embodiments, R² includes a nucleobase that is xanthine or a derivative thereof. In embodiments, R² includes a nucleobase that is 7-methylguanine or a derivative thereof. In embodiments, R² includes a nucleobase that is 5,6-dihydrouracil or a derivative thereof. In embodiments, R² includes a nucleobase that is 5-methylcytosine or a derivative thereof. In embodiments, R² includes a nucleobase that is or 5-hydroxymethylcytosine or a derivative thereof. In embodiments, R² includes a monovalent nucleobase. In embodiments, R² includes a divalent nucleobase. In an embodiment, the nucleoside is a deoxyribonucleoside. In another embodiment, the nucleoside is a ribonucleoside. In embodiments, R² includes modifications in the nucleobase and/or sugar.

In embodiments, R² is a polymer moiety composed of repeating subunits. In embodiments, R² is a polymer moiety composed of amino acid residues. In embodiments, R² is a polymer moiety composed of naturally occurring, non-natural amino acid residues, or a combination thereof. In embodiments, R² is a polymer moiety composed of nucleic acids in single- or double-stranded form.

In embodiments, R² is a biomolecule that is capable of targeting a biomolecule-specific binding moiety in or on a cell to facilitate the delivery of the compound described herein. In embodiments, the biomolecule-specific binding moiety is an extracellular target. In embodiments, the biomolecule-specific binding moiety is an intracellular target. In embodiments, the biomolecule-specific binding moiety is a cell surface receptor. In embodiments, the biomolecule described herein is capable of targeting an antibody. In embodiments, the biomolecule described herein is capable of targeting a protein. In embodiments, the biomolecule described herein is capable of targeting a polynucleotide. In embodiments, the biomolecule described herein is capable of targeting an oligonucleotide. In embodiments, the biomolecule described herein is capable of targeting a DNA molecule. In embodiments, the biomolecule described herein is capable of targeting a RNA molecule. In embodiments, the biomolecule described herein is capable of targeting a guide RNA molecule. In embodiments, the biomolecule is capable of targeting a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, a carbohydrate, or a sterol moiety.

In embodiments, R² is a nanoparticle. In embodiments, R² is a nanoparticle with a metal nanoparticle core. In embodiments, R² is a nanoparticle with an organic nanoparticle core. In embodiments, R² is a nanoparticle with a silicon nanoparticle core. In embodiments, R² is a nanoparticle with a boron nanoparticle core. In embodiments, R² is a lipid-based nanoparticle, inorganic nanoparticle, or polymeric nanoparticle. In embodiments, a lipid-based nanoparticle is a liposome, lipid nanoparticle, or an emulsion. In embodiments, an inorganic nanoparticle is a silica nanoparticle, quantum dot nanoparticle, iron oxide nanoparticle, or a gold nanoparticle. In embodiments, a polymeric nanoparticle is a polymersome, dendrimer, polymeric micelle, or a nanosphere.

In embodiments, R² is a detectable agent moiety. In embodiments, R² is a radioactive substance moiety. In embodiments, R² is a detectable agent moiety containing ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. In embodiments, R² is a detectable agent moiety containing a paramagnetic ion from the transition and lanthanide metal groups.

In embodiments, R² is a chelating moiety formed from a detectable agent moiety and a chelating agent. In embodiments, R² is a chelating moiety formed from the chelating agent, DOTA (1,4,7,10-tetra-azacyclododecane-1,4,7,10-tetraacetic acid), and the following metals: ¹¹¹In, ¹⁷⁷Lu, ^{86/90}Y ²²⁵Ac, ⁴⁴/4⁷Sc, or ⁶⁸Ga. In embodiments, R² is a chelate formed from DOTA and ¹⁷⁷Lu. In embodiments, R² is a chelate formed from NOTA (1,4,7-triazacyclononane-1,4,7-triacetic acid) and the following metals: ^{67/68}Ga, ⁶⁴Cu, ¹⁷⁷Lu, ^{86/90}Y, or ^{212/213}Bi. In embodiments, R² is a chelate formed from TETA (1,4,8,11-tetra-azacyclotetradecane-1,4,8,11-tetraacetic acid) and ⁶⁴Cu. In embodiments, R² is a chelate formed from DTPA (diethylenetriaminepentaacetic acid) and ¹¹¹In or ¹⁷⁷Lu.

In embodiments, R² is a detectable agent. In embodiments, R² is fluorescent or luminescent substance or molecule. In embodiments, R² is a fluorescent moiety or fluorescent dye moiety. In embodiments, the R² is a fluorescent molecule (e.g., acridine dye, cyanine, dye, fluorine dye, oxazine dye, phenanthridine dye, or rhodamine dye). In embodiments, the R² is a fluorescent molecule (e.g., acridine dye, cyanine, dye, fluorine dye, oxazine dye, phenanthridine dye, or rhodamine dye). In embodiments, the R² is a fluorescent moiety or fluorescent dye moiety. In embodiments, the R² is a Janelia Fluor® dye moiety, naphthalimide moiety, fluorescein isothiocyanate moiety, tetramethylrhodamine-5-(and 6)-isothiocyanate moiety, Cy®2 moiety, Cy®3 moiety, Cy®5 moiety, Cy®7 moiety, 4′,6-diamidino-2-phenylindole moiety, Hoechst 33258 moiety, Hoechst 33342 moiety, Hoechst 34580 moiety, propidium-iodide moiety, or acridine orange moiety. In embodiments, the R² is a Indo-1, Ca saturated moiety, Indo-1 Ca²⁺ moiety, Cascade Blue® BSA pH 7.0 moiety, Cascade Blue® moiety, LysoTracker® Blue moiety, Alexa Fluor® 405 moiety, LysoSensor® Blue pH 5.0 moiety, LysoSensor® Blue moiety, DyLight™ 405 moiety, DyLight™ 350 moiety, BFP (Blue Fluorescent Protein) moiety, Alexa Fluor® 350 moiety, 7-Amino-4-methylcoumarin pH 7.0 moiety, Amino Coumarin moiety, AMCA conjugate moiety, Coumarin moiety, 7-Hydroxy-4-methylcoumarin moiety, 7-Hydroxy-4-methylcoumarin pH 9.0 moiety, 6,8-Difluoro-7-hydroxy-4-methylcoumarin pH 9.0 moiety, Hoechst 33342 moiety, Pacific Blue™ moiety, Hoechst 33258 moiety, Hoechst 33258-DNA moiety, Pacific Blue™ antibody conjugate pH 8.0 moiety, PO-PRO™-1 moiety, PO-PRO™-1-DNA moiety, POPO™-1 moiety, POPO™-1-DNA moiety, DAPI-DNA moiety, DAPI moiety, Marina Blue® moiety, SYTOX™ Blue-DNA moiety, CFP (Cyan Fluorescent Protein) moiety, eCFP (Enhanced Cyan Fluorescent Protein) moiety, 1-Anilinonaphthalene-8-sulfonic acid (1,8-ANS) moiety, Indo-1, Ca free moiety, 1,8-ANS (1-Anilinonaphthalene-8-sulfonic acid) moiety, BO-PRO™-1-DNA moiety, BOPRO™-1 moiety, BOBO™-1-DNA moiety, SYTO™ 45-DNA moiety, evoglow-Pp1 moiety, evoglow-Bs1 moiety, evoglow-Bs2 moiety, Auramine O moiety, DiO moiety, LysoSensor® Green pH 5.0 moiety, Cy® 2 moiety, LysoSensor® Green moiety, Fura-2, high Ca moiety, Fura-2 Ca²⁺ moiety, SYTO™ 13-DNA moiety, YO-PRO™-1-DNA moiety, YOYO™-1-DNA moiety, eGFP (Enhanced Green Fluorescent Protein) moiety, LysoTracker® Green moiety, GFP (S65T) moiety, BODIPY® FL, MeOH moiety, Sapphire moiety, BODIPY® FL conjugate moiety, MitoTracker™ Green moiety, MitoTracker™ Green FM, Fluorescein 0.1 M NaOH moiety, Calcein pH 9.0 moiety, Fluorescein pH 9.0 moiety, Calcein moiety, Fluo-4 moiety, FDA moiety, DTAF moiety, Fluorescein moiety, CFDA moiety, FITC moiety, Alexa Fluor® 488 hydrazide-water moiety, DyLight™ 488 moiety, 5-FAM pH 9.0 moiety, Alexa Fluor® 488 moiety, Rhodamine 110 moiety, Rhodamine 110 pH 7.0 moiety, Acridine Orange moiety, BCECF pH 5.5 moiety, PicoGreen® dsDNA quantitation reagent moiety, SYBR® Green I moiety, Rhodamine Green pH 7.0 moiety, CyQUANT™ GR-DNA moiety, NeuroTrace™ 500/525, green fluorescent Nissl stain-RNA moiety, DansylCadaverine moiety, Fluoro-Emerald moiety, Nissl moiety, Fluorescein dextran pH 8.0 moiety, Rhodamine Green moiety, 5-(and-6)-Carboxy-2′,7′-dichlorofluorescein pH 9.0 moiety, DansylCadaverine, eYFP (Enhanced Yellow Fluorescent Protein) moiety, Oregon Green™ 488 moiety, Fluo-3 moiety, BCECF pH 9.0 moiety, SBFI-Na⁺ moiety, Fluo-3 Ca²⁺ moiety, Rhodamine 123 MeOH moiety, FlAsH moiety, Calcium Green-1 Ca²⁺ moiety, Magnesium Green moiety, DM-NERF pH 4.0 moiety, Calcium Green moiety, Citrine moiety, LysoSensor® Yellow pH 9.0 moiety, TO-PRO™-1-DNA moiety, Magnesium Green Mg²⁺ moiety, Sodium Green Na⁺ moiety, TOTO™-1-DNA moiety, Oregon Green™ 514 moiety, Oregon Green™ 514 antibody conjugate pH 8.0 moiety, NBD-X moiety, DM-NERF pH 7.0 moiety, NBD-X, MeOH moiety, CI-NERF pH 6.0 moiety, Alexa Fluor® 430 moiety, CI-NERF pH 2.5 moiety, Lucifer Yellow, CH moiety, LysoSensor® Yellow pH 3.0 moiety, 6-TET, SE pH 9.0 moiety, Eosin antibody conjugate pH 8.0 moiety, Eosin moiety, 6-Carboxyrhodamine 6G pH 7.0 moiety, 6-Carboxyrhodamine 6G hydrochloride moiety, BODIPY® R6G SE moiety, BODIPY® R6G MeOH moiety, 6 JOE moiety, Cascade Yellow® moiety, mBanana moiety, Alexa Fluor® 532 moiety, Erythrosin-5-isothiocyanate pH 9.0 moiety, 6-HEX, SE pH 9.0 moiety, mOrange moiety, mHoneydew moiety, Cy®3 moiety, Rhodamine B moiety, DiI moiety, 5-TAMRA™-MeOH moiety, Alexa Fluor® 555 moiety, DyLight™ 549 moiety, BODIPY® TMR-X, SE moiety, BODIPY® TMR-X MeOH moiety, PO-PRO™-3-DNA moiety, PO-PRO™-3 moiety, Rhodamine moiety, POPO™-3 moiety, Alexa Fluor® 546 moiety, Calcium Orange Ca²⁺ moiety, TRITC moiety, Calcium Orange moiety, Rhodaminephalloidin pH 7.0 moiety, MitoTracker™ Orange moiety, MitoTracker™ Orange MeOH moiety, Phycoerythrin moiety, Magnesium Orange moiety, R-Phycoerythrin pH 7.5 moiety, 5-TAMRA™ pH 7.0 moiety, 5-TAMRA™ moiety, Rhod-2 moiety, FM® 1-43 moiety, Rhod-2 Ca²⁺ moiety, FM® 1-43 lipid moiety, LOLO™-1-DNA moiety, dTomato moiety, DsRed moiety, Dapoxyl (2-aminoethyl) sulfonamide moiety, Tetramethylrhodamine dextran pH 7.0 moiety, Fluor-Ruby moiety, Resorufin moiety, Resorufin pH 9.0 moiety, mTangerine moiety, LysoTracker® Red moiety, Lissaminerhodamine moiety, Cy® 3.5 moiety, Rhodamine Red-X antibody conjugate pH 8.0 moiety, Sulforhodamine 101 EtOH moiety, JC-1 pH 8.2 moiety, JC-1 moiety, mStrawberry moiety, MitoTracker™ Red moiety, MitoTracker™ Red, MeOH moiety, X-Rhod-1 Ca²⁺ moiety, Alexa Fluor® 568 moiety, 5-ROX pH 7.0 moiety, 5-ROX (5-Carboxy-X-rhodamine, triethylammonium salt) moiety, BO-PRO™-3-DNA moiety, BOPRO™-3 moiety, BOBO™-3-DNA moiety, Ethidium Bromide moiety, ReAsH moiety, Calcium Crimson moiety, Calcium Crimson Ca²⁺ moiety, mRFP moiety, mCherry moiety, HcRed moiety, DyLight™ 594 moiety, Ethidium homodimer-1-DNA moiety, Ethidiumhomodimer moiety, Propidium Iodide moiety, SYPRO® Ruby moiety, Propidium Iodide-DNA moiety, Alexa Fluor® 594 moiety, BODIPY® TR-X, SE moiety, BODIPY® TR-X, MeOH moiety, BODIPY® TR-X phallacidin pH 7.0 moiety, Alexa Fluor® 610 R-phycoerythrin streptavidin pH 7.2 moiety, YO-PRO™-3-DNA moiety, Di-8 ANEPPS moiety, Di-8-ANEPPS-lipid moiety, YOYO™-3-DNA moiety, Nile Red-lipid moiety, Nile Red moiety, DyLight™ 633 moiety, mPlum moiety, TO-PRO-3-DNA moiety, DDAO pH 9.0 moiety, Fura Red high Ca moiety, Allophycocyanin pH 7.5 moiety, APC (allophycocyanin) moiety, Nile Blue, TOTO-3-DNA moiety, Cy® 5 moiety, BODIPY® 650/665-X MeOH moiety, Alexa Fluor® 647 R-phycoerythrin streptavidin pH 7.2 moiety, DyLight™ 649 moiety, Alexa Fluor® 647 moiety, Fura Red Ca²⁺ moiety, ATTO™ 647 moiety, Fura Red, low Ca moiety, Carboxynaphthofluorescein pH 10.0 moiety, Alexa Fluor® 660 moiety, Cy® 5.5 moiety, Alexa Fluor® 680 moiety, DyLight™ 680 moiety, Alexa Fluor® 700 moiety, FM® 4-64, 2% CHAPS moiety, or FM® 4-64 moiety. In embodiments, the detectable moiety is a moiety of 1,1-Diethyl-4,4-carbocyanine iodide, 1,2-Diphenylacetylene, 1,4-Diphenylbutadiene, 1,4-Diphenylbutadiyne, 1,6-Diphenylhexatriene, 1,6-Diphenylhexatriene, 1-anilinonaphthalene-8-sulfonic acid, 2,7-Dichlorofluorescein, 2,5-DIPHENYLOXAZOLE, 2-Di-1-ASP, 2-dodecylresorufin, 2-Methylbenzoxazole, 3,3-Diethylthiadicarbocyanine iodide, 4-Dimethylamino-4-Nitrostilbene, 5(6)-Carboxyfluorescein, 5(6)-Carboxynaphtofluorescein, 5(6)-Carboxytetramethylrhodamine B, 5-(and-6)-carboxy-2′,7′-dichlorofluorescein, 5-(and-6)-carboxy-2,7-dichlorofluorescein, 5-(N-hexadecanoyl)aminoeosin, 5-(N-hexadecanoyl)aminoeosin, 5-chloromethylfluorescein, 5-FAM, 5-ROX™, 5-TAMRA™, 6,8-difluoro-7-hydroxy-4-methylcoumarin, 6-carboxyrhodamine 6G, 6-HEX, 6-JOE, 6-JOE, 6-TET, 7-aminoactinomycin D, 7-Benzylamino-4-Nitrobenz-2-Oxa-1,3-Diazole, 7-Methoxycoumarin-4-Acetic Acid, 8-Benzyloxy-5,7-diphenylquinoline, 8-Benzyloxy-5,7-diphenylquinoline, 9,10-Bis(Phenylethynyl)Anthracene, 9,10-Diphenylanthracene, 9-METHYLCARBAZOLE, (CS)2Ir(μ-Cl)2Ir(CS)2, AAA, Acridine Orange, Acridine Yellow, Adams Apple Red 680, Adirondack Green 520, Alexa Fluor® 350, Alexa Fluor® 405, Alexa Fluor® 430, Alexa Fluor® 480, Alexa Fluor® 488, Alexa Fluor® 488 hydrazide, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 610-R-PE, Alexa Fluor® 633, Alexa Fluor® 635, Alexa Fluor® 647, Alexa Fluor® 647-R-PE, Alexa Fluor® 660, Alexa Fluor® 680, Alexa Fluor® 680-APC, Alexa Fluor® 680-R-PE, Alexa Fluor® 700, Alexa Fluor® 750, Alexa Fluor® 790, Allophycocyanin, AmCyan1, Aminomethylcoumarin, Amplex Gold (product), Amplex Red Reagent, Amplex UltraRed, Anthracene, APC, APC-Seta-750, AsRed2, ATTO™ 390, ATTO™ 425, ATTO™ 430LS, ATTO™ 465, ATTO™ 488, ATTO™ 490LS, ATTO™ 495, ATTO™ 514, ATTO™ 520, ATTO™ 532, ATTO™ 550, ATTO™ 565, ATTO™ 590, ATTO™ 594, ATTO™ 610, ATTO™ 620, ATTO™ 633, ATTO™ 635, ATTO™ 647, ATTO™ 647N, ATTO™ 655, ATTO™ 665, ATTO™ 680, ATTO™ 700, ATTO™ 725, ATTO™ 740, ATTO™ Oxa12, ATTO™ Rho3B, ATTO™ Rho6G, ATTO™ Rho11, ATTO™ Rhol2, ATTO™ Rhol3, ATTO™ Rhol4, ATTO™ Rhol01, ATTO™ Thio12, Auramine O, Azami Green, Azami Green monomeric, B-phycoerythrin, BCECF, BCECF, Bexi, Biphenyl, Birch Yellow 580, Blue-green algae, BO-PRO™-1, BO-PRO™-3, BOBO™-1, BOBO™-3, BODIPY® 630 650-X, BODIPY® 650/665-X, BODIPY® FL, BODIPY® R6G, BODIPY® TMR-X, BODIPY® TR-X, BODIPY® TR-X Ph 7.0, BODIPY® TR-X phallacidin, BODIPY®-DiMe, BODIPY®-Phenyl, BODIPY®-TMSCC, C3-Indocyanine, C3-Indocyanine, C3-Oxacyanine, C3-Thiacyanine Dye (EtOH), C3-Thiacyanine Dye (PrOH), C5-Indocyanine, C5-Oxacyanine, C5-Thiacyanine, C7-Indocyanine, C7-Oxacyanine, C545T, C-Phycocyanin, Calcein, Calcein red-orange, Calcium Crimson, Calcium Green-1, Calcium Orange, Calcofluor white 2MR, Carboxy SNARF-1 pH 6.0, Carboxy SNARF-1 pH 9.0, Carboxynaphthofluorescein, Cascade Blue®, Cascade Yellow, Catskill Green 540, CBQCA, CellMask™ Orange, CellTrace BODIPY TR methyl ester, CellTrace calcein violet, CellTrace™ Far Red, CellTracker™ Blue, CellTracker™ Red CMTPX, CellTracker™ Violet BMQC, CF®405M, CF®405S, CF®488A, CF®543, CF®555, CFP, CFSE, CF™ 350, CF™ 485, Chlorophyll A, Chlorophyll B, Chromeo™ 488, Chromeo™ 494, Chromeo™ 505, Chromeo™ 546, Chromeo™ 642, Citrine, ClOH butoxy aza-BODIPY®, ClOH C12 aza-BODIPY®, CM-H2DCFDA, Coumarin 1, Coumarin 6, Coumarin 6, Coumarin 30, Coumarin 314, Coumarin 334, Coumarin 343, Coumarine 545T, Cresyl Violet Perchlorate, CryptoLight CF1, CryptoLight CF2, CryptoLight CF3, CryptoLight CF4, CryptoLight CF5, CryptoLight CF6, Crystal Violet, Coumarin153, Cy®2, Cy®3, Cy®3.5, Cy®3B, Cy®3Cy®5 ET, Cy®5, Cy®5.5, Cy®7, Cyanine3 NHS ester, Cyanine5 carboxylic acid, Cyanine5 NHS ester, Cyclotella meneghiniana Kützing, CypHer5, CypHer5 pH 9.15, CyQUANT™ GR, CyTrak Orange™, Dabcyl SE, DAF-FM, DAMC (Weiss), dansyl cadaverine, Dansyl Glycine (Dioxane), Dapoxyl (2-aminoethyl)sulfonamide, DDAO, Deep Purple, di-8-ANEPPS, DiA, Dichlorotris(1,10-phenanthroline) ruthenium(II), DiClOH C12 aza-BODIPY®, DiClOHbutoxy aza-BODIPY®, DiD, DiI, DiIC18(3), DiO, DiR, Diversa Cyan-FP, Diversa Green-FP, DM-NERF pH 4.0, DOCI, Doxorubicin, DPP pH-Probe 590-7.5, DPP pH-Probe 590-9.0, DPP pH-Probe 590-11.0, DPP pH-Probe 590-11.0, Dragon Green, DRAQ5™, DsRed, DsRed-Express, DsRed-Express2, DsRed-Express T1, dTomato, DY-350XL, DY-480, DY-480XL MegaStokes, DY-485, DY-485XL MegaStokes, DY-490, DY-490XL MegaStokes, DY-500, DY-500XL MegaStokes, DY-520, DY-520XL MegaStokes, DY-547, DY-549P1, DY-554, DY-555, DY-557, DY-590, DY-615, DY-630, DY-631, DY-633, DY-635, DY-636, DY-647, DY-649P1, DY-650, DY-651, DY-656, DY-673, DY-675, DY-676, DY-680, DY-681, DY-700, DY-701, DY-730, DY-731, DY-750, DY-751, DY-776, DY-782, Dye-28, Dye-33, Dye-45, Dye-304, Dye-1041, DyLight® 488, DyLight® 549, DyLight® 633, DyLight® 649, DyLight® 680, E2-Crimson, E2-Orange, E2-Red/Green, EBFP, ECF, ECFP, ECL Plus, eGFP, ELF 97, Emerald, Envy Green, Eosin, Eosin Y, epicocconone, EqFP611, Erythrosin-5-isothiocyanate, Ethidium bromide, ethidium homodimer-1, Ethyl Eosin, Ethyl Nile Blue A, Ethyl-p-Dimethylaminobenzoate, Ethyl-p-Dimethylaminobenzoate, Eu2O3 nanoparticles, Eu (Soini), Eu(tta)3DEADIT, EvaGreen®, EVOblue-30, EYFP, FAD, FITC, FITC, FlAsH (Adams), Flash Red EX, FlAsH-CCPGCC, FlAsH-CCXXCC, Fluo-3, Fluo-4, Fluo-5F, Fluorescein, Fluorescein 0.1 NaOH, Fluorescein-Dibase, fluoro-emerald, Fluorol 5G, FluoSpheres® blue, FluoSpheres® crimson, FluoSpheres® dark red, FluoSpheres® orange, FluoSpheres® red, FluoSpheres® yellow-green, FM®4-64 in CTC, FM®4-64 in SDS, FM® 1-43, FM® 4-64, Fort Orange 600, Fura Red, Fura Red Ca free, fura-2, Fura-2 Ca free, Gadodiamide, Gd-Dtpa-Bma, Gadodiamide, Gd-Dtpa-Bma, GelGreen™, Ge1Red™, H9-40, HcRed1, Hemo Red 720, HiLyte™ Fluor 488, HiLyte™ Fluor 555, HiLyte™ Fluor 647, HiLyte™ Fluor 680, HiLyte™ Fluor 750, HiLyte™ Plus 555, HiLyte™ Plus 647, HiLyte™ Plus 750, HmGFP, Hoechst 33258, Hoechst 33342, Hops Yellow 560, HPTS, indo-1, Indo-1 Ca free, Ir(Cn)2(acac), Ir(Cs)2(acac), IR-775 chloride, IR-806, Ir-OEP-CO-Cl, IRDye® 650 Alkyne, IRDye® 650 Azide, IRDye® 650 Carboxylate, IRDye® 650 DBCO, IRDye® 650 Maleimide, IRDye® 650 NHS Ester, IRDye® 680LT Carboxylate, IRDye® 680LT Maleimide, IRDye® 680LT NHS Ester, IRDye® 680RD Alkyne, IRDye® 680RD Azide, IRDye® 680RD Carboxylate, IRDye® 680RD DBCO, IRDye® 680RD Maleimide, IRDye® 680RD NHS Ester, IRDye® 700 phosphoramidite, IRDye® 700DX, IRDye® 700DX Carboxylate, IRDye® 700DX NHS Ester, IRDye® 750 Carboxylate, IRDye® 750 Maleimide, IRDye® 750 NHS Ester, IRDye® 800 phosphoramidite, IRDye® 800CW, IRDye® 800CW Alkyne, IRDye® 800CW Azide, IRDye® 800CW Carboxylate, IRDye® 800CW DBCO, IRDye® 800CW Maleimide, IRDye® 800CW NHS Ester, IRDye® 800RS, IRDye® 800RS Carboxylate, IRDye® 800RS NHS Ester, IRDye® QC-1 Carboxylate, IRDye® QC-1 NHS Ester, JC-1, JOJO™-1, Jonamac Red Evitag T2, Kaede Green, Kaede Red, kusabira orange, Lake Placid 490, LDS 751, Lissamine Rhodamine (Weiss), LOLO™-1, Lucifer Yellow CH, Lucifer Yellow CH Dilitium salt, Lumio Green, Lumio Red, Lumogen® F Orange, Lumogen® Red F300, Lumogen® Red F300, LysoSensor® Blue DND-192, LysoSensor® Green DND-153, LysoSensor® Green DND-153, LysoSensor® Yellow/Blue DND-160 pH 3, LysoSensor® YellowBlue DND-160, LysoTracker® Blue DND-22, LysoTracker® Green DND-26, LysoTracker® Red DND-99, LysoTracker® Yellow HCK-123, Macoun Red Evitag T2, Macrolex® Fluorescence Red G, Macrolex® Fluorescence Yellow 10GN, Macrolex® Fluorescence Yellow 10GN, Magnesium Green, Magnesium Octaethylporphyrin, Magnesium Orange, Magnesium Phthalocyanine, Magnesium Tetramesitylporphyrin, Magnesium Tetraphenylporphyrin, malachite green isothiocyanate, Maple Red-Orange 620, Marina Blue®, mBanana, mBBr, mCherry, Merocyanine 540, Methyl green, Methylene Blue, mHoneyDew, MitoTracker™ Deep Red 633, MitoTracker™ Green FM, MitoTracker™ Orange CMTMRos, MitoTracker™ Red CMXRos, monobromobimane, Monochlorobimane, Monoraphidium, mOrange, mOrange2, mPlum, mRaspberry, mRFP, mRFP1, mRFP1.2 (Wang), mStrawberry (Shaner), mTangerine (Shaner), N,N-Bis(2,4,6-trimethylphenyl)-3,4:9,10-perylenebis(dicarboximide), Naphthalene, Naphthofluorescein, Naphthofluorescein, NBD-X, NeuroTrace™ 500525, Nilblau perchlorate, Nile Blue (EtOH), Nile red, Nileblue A, NIR1, NIR2, NIR3, NIR4, NIR820, Octaethylporphyrin, OH butoxy aza-BODIPY®, OHC12 aza-BODIPY®, Orange Fluorescent Protein, Oregon Green™ 488, Oregon Green™ 488 DHPE, Oregon Green™ 514, Oxazin1, Oxazin 750, Oxazine 1, Oxazine 170, P4-3, P-Quaterphenyl, P-Terphenyl, PA-GFP (post-activation), PA-GFP (pre-activation), Pacific Orange, Palladium(II) meso-tetraphenyl-tetrabenzoporphyrin, PdOEPK, PdTFPP, PerCP-Cy®5.5, Perylene, Perylene bisimide pH-Probe 550-5.0, Perylene bisimide pH-Probe 550-5.5, Perylene bisimide pH-Probe 550-6.5, Perylene Green pH-Probe 720-5.5, Perylene Green Tag pH-Probe 720-6.0, Perylene Orange pH-Probe 550-2.0, Perylene Orange Tag 550, Perylene Red pH-Probe 600-5.5, Perylenediimid, Perylne Green pH-Probe 740-5.5, Phenol, Phenylalanine, pHrodo, succinimidyl ester, Phthalocyanine, PicoGreen® dsDNA quantitation reagent, Pinacyanol-Iodide, Piroxicam, Platinum(II) tetraphenyltetrabenzoporphyrin, Plum Purple, PO-PRO™-1, PO-PRO™-3, POPO™-1, POPO™-3, POPOP, Porphin, Proflavin, PromoFluor-350, PromoFluor-405, PromoFluor-415, PromoFluor-488, PromoFluor-488 Premium, PromoFluor-488LSS, PromoFluor-500LSS, PromoFluor-505, PromoFluor-510LSS, PromoFluor-514LSS, PromoFluor-520LSS, PromoFluor-532, PromoFluor-546, PromoFluor-555, PromoFluor-590, PromoFluor-610, PromoFluor-633, PromoFluor-647, PromoFluor-670, PromoFluor-680, PromoFluor-700, PromoFluor-750, PromoFluor-770, PromoFluor-780, PromoFluor-840, Protoporphyrin IX, PTIR475/UF, PTIR545/UF, PtOEP, PtOEPK, PtTFPP, Pyrene, QD525, QD565, QD585, QD605, QD655, QD705, QD800, QD903, QD PbS 950, QDot™ 525, QDot™ 545, QDot™ 565, Qdot™ 585, Qdot™ 605, Qdot™ 625, Qdot™ 655, Qdot™ 705, Qdot™ 800, QpyMe2, QSY 7, QSY 7, QSY 9, QSY 21, QSY 35, quinine, Quinine sulfate, R-phycoerythrin, ReAsH-CCPGCC, ReAsH-CCXXCC, Red Beads (Weiss), Redmond Red, Resorufin, rhod-2, Rhodamin 700 perchlorate, rhodamine, Rhodamine 6G, Rhodamine 101, rhodamine 110, Rhodamine 123, rhodamine 123, Rhodamine B, Rhodamine Green, Rhodamine pH-Probe 585-7.0, Rhodamine pH-Probe 585-7.5, Rhodamine phalloidin, Rhodamine Red-X, Rhodamine Tag pH-Probe 585-7.0, Rhodol Green, Riboflavin, Rose Bengal, Sapphire, SBFI, SBFI Zero Na, Scenedesmus sp., SensiLight PBXL-1, SensiLight PBXL-3, Seta 633-NHS, Seta-633-NHS, SeTau-380-NHS, SeTau-647-NHS, Snake-Eye Red 900, SNIR1, SNIR2, SNIR3, SNIR4, Sodium Green, Solophenyl flavine 7GFE 500, Spectrum Aqua, Spectrum Blue, Spectrum FRed, Spectrum Gold, Spectrum Green, Spectrum Orange, Spectrum Red, Squarylium dye III, Stains All, Stilben derivate, Stilbene, Styry18 perchlorate, Sulfo-Cyanine3 carboxylic acid, Sulfo-Cyanine3 NHS ester, Sulfo-Cyanine5 carboxylic acid, Sulforhodamine 101, Sulforhodamine B, Sulforhodamine G, Suncoast Yellow, SuperGlo BFP, SuperGlo GFP, Surf Green EX, SYBR Gold nucleic acid gel stain, SYBR® Green I, SYPRO® Ruby, SYTO™ 9, SYTO™ 11, SYTO™ 13, SYTO™ 16, SYTO™ 17, SYTO™ 45, SYTO™ 59, SYTO™ 60, SYTO™ 61, SYTO™ 62, SYTO™ 82, SYTO™ RNASelect, SYTO™ RNASelect, SYTOX™ Blue, SYTOX™ Green, SYTOX™ Orange, SYTOX™ Red, T-Sapphire, CF® Dye TCO, tdTomato, Terrylen, Terrylendiimid, Tetra-t-Butylazaporphine, Tetra-t-Butylnaphthalocyanine, Tetracen, Tetrakis(o-Aminophenyl)Porphyrin, Tetramesitylporphyrin, Tetramethylrhodamine, Tetraphenylporphyrin, Texas Red, Texas Red DHPE, Texas Red-X, ThiolTracker Violet, Thionin acetate, TMRE, TO-PRO™-1, TO-PRO™-3, Toluene, Topaz (Tsien1998), TOTO™-1, TOTO™-3, Tris(2,2-Bipyridyl)Ruthenium(II) chloride, Tris(4,4-diphenyl-2,2-bipyridine) ruthenium(II) chloride, Tris(4,7-diphenyl-1,10-phenanthroline) ruthenium(II) TMS, TRITC (Weiss), TRITC Dextran (Weiss), Tryptophan, Tyrosine, Vex1, Vybrant™ DyeCycle Green stain, Vybrant™ DyeCycle Orange stain, Vybrant™ DyeCycle Violet stain, WEGFP (post-activation), WellRED D2, WellRED D3, WellRED D4, WtGFP, WtGFP (Tsien1998), X-rhod-1, Yakima Yellow, YFP, YO-PRO™-1, YO-PRO™-3, YOYO™-1, YoYo™-1, YoYo™-1 dsDNA, YoYo™-1 ssDNA, YOYO™-3, Zinc Octaethylporphyrin, Zinc Phthalocyanine, Zinc Tetramesitylporphyrin, Zinc Tetraphenylporphyrin, ZsGreen1, or ZsYellow1. In embodiments, the detectable moiety is a moiety of a derivative of one of the detectable moieties described immediately above, wherein the derivative differs from one of the detectable moieties immediately above by a modification resulting from the conjugation of the detectable moiety to the compound described herein. Janelia Fluor® is a registered trademark of Howard Hughes Medical Institute. Cascade Blue®, SYPRO®, and Oregon Green® are registered trademarks of Life Technologies. LysoTracker™, FluoSpheres™, FM™, Fura Red™, LysoSensor®, SYBR®, TO-PRO®, TOTO™, and Marina Blue® are trademarks of Invitrogen. Pacific Blue™, PO-PRO®, POPO®, SYTOX Blue™, BO-PRO™, BOBO™, YO-PRO™ YOYO™ MitoTracker™, PicoGreen®, NeuroTrace™, Fura Red™, CellTrace™, CellMask™ LOLO™-1, JOJO™-1, Qdot™, QSY™, CyQUANT™, DyLight® dyes, SYTO™, and SYTOX Blue™ are trademarks of Thermo Fisher, BODIPY® is a registered trademark of Molecular Probes, TAMRA™ is a trademark of Appelera, Chromeo™ is a trademark of Active Motif Chromeon GmbH, CyTRACK Orange™ and DRAQ5™ are trademarks of Biostatus Limited, EvaGreen®, GelGreen®, GelRed®, CF®, and FM™ are trademarks of Biotium. Macrolex® is a trademark of Lanxess, SpectrumFRed™, SpectrumRed™, SpectrumGold™, SpectrumOrange™, SpectrumGreen™, SpectrumAqua™, and SpectrumBlue™ Series Vysis™ SpectrumFRed™, SpectrumRed™, SpectrumGold™, SpectrumOrange™, SpectrumGreen™, SpectrumAqua™ and SpectrumBlue™ are trademarks of Abbott Molecular Inc. HiLyte™ is a trademark of Anaspec, Inc. IRDye® is a trademark of Li-Cor Biosciences, Inc. Rox™ is a trademark of Applied Biosystems, Atto™ is a trademark of ATTO-TEC GmbH. Cy® is a registered trademark of Cytiva.

In embodiments, the detectable agent has a maximum excitation wavelength between 350-400 nm, between 400-450 nm, between 450-500 nm, between 500-550 nm, between 550-600 nm, between 600-650 nm, between 650-700 nm, 700-750 nm, or between 750-800 nm. In embodiments, the detectable agent has a maximum excitation wavelength of about 325 nm, 343 nm, 350 nm, 353 nm, 359 nm, 360 nm, 395 nm, 400 nm, 401 nm, 402 nm, 403 nm, 425 nm, 434 nm, 440 nm, 466 nm, 480 nm, 485 nm, 489 nm, 490 nm, 492 nm, 493 nm, 494 nm, 495 nm, 496 nm, 498 nm, 499 nm, 500 nm, 502 nm, 503 nm, 505 nm, 517 nm, 518 nm, 520 nm, 525 nm, 528 nm, 530 nm, 531 nm, 535 nm, 542 nm, 544 nm, 547 nm, 550 nm, 553 nm, 554 nm, 558 nm, 560 nm, 561 nm, 562 nm, 565 nm, 567 nm, 570 nm, 572 nm, 579 nm, 581 nm, 589 nm, 590 nm, 591 nm, 593 nm, 596 nm, 610 nm, 631 nm, 632 nm, 638 nm, 650 nm, 652 nm, 654 nm, 663 nm, 675 nm, 680 nm, 692 nm, 696 nm, 743 nm, 752 nm, 777 nm, or 782 nm.

In embodiments, the detectable agent has a maximum emission wavelength between 400-450 nm, between 450-500 nm, between 500-550 nm, between 550-600 nm, between 600-650 nm, between 650-700 nm, between 700-750 nm, between 750-800 nm, or between 800-850 nm. In embodiments, the detectable agent has a maximum emission of about 410 nm, 420 nm, 421 nm, 423 nm, 432 nm, 442 nm, 445 nm, 455 nm, 506 nm, 512 nm, 514 nm, 517 nm, 518 nm, 519 nm, 520 nm, 521 nm, 523 nm, 525 nm, 528 nm, 533 nm, 537 nm, 539 nm, 540 nm, 542 nm, 548 nm, 550 nm, 551 nm, 554 nm, 555 nm, 556 nm, 565 nm, 568 nm, 570 nm, 572 nm, 573 nm, 574 nm, 575 nm, 576 nm, 578 nm, 580 nm, 590 nm, 591 nm, 594 nm, 595 nm, 596 nm, 603 nm, 605 nm, 613 nm, 615 nm, 617 nm, 618 nm, 619 nm, 620 nm, 629 nm, 630 nm, 640 nm, 647 nm, 648 nm, 658 nm, 660 nm, 668 nm, 670 nm, 673 nm, 675 nm, 691 nm, 694 nm, 695 nm, 702 nm, 712 nm, 719 nm, 767 nm, 776 nm, 778 nm, 794 nm, or 804 nm.

In embodiments, R² is a tropic cell. In embodiments, R² is an embryonic stem cell (ESC). In embodiments, R² is an embryonic germ cell (ESG). In embodiments, R² is an induced pluripotent stem cell (iPSC). In embodiments, R² is an embryonic carcinoma cell (ECC). In embodiments, R² is a bone marrow stem cell. In embodiments, R² is an adult stem cell. In embodiments, R² is a hematopoietic stem cell. In embodiments, R² is a neural stem cell or mesenchymal stem cell.

In embodiments, R² is a neural stem cell. In embodiments, R² is mesenchymal stem cell. In embodiments, R² is a mesenchymal stromal cell. In embodiments, R² is a hematopoietic stem cell. In embodiments, R² is an adoptively transferred T-lymphocyte. In embodiments, R² is macrophage. In embodiments, R² is a liver stem cell. In embodiments, R² is an embryoid body.

In embodiments, R³ is a substituted (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group. In embodiments, when R³ is substituted, it is substituted with 1 to 10 substituent groups. In embodiments, when R³ is substituted, it is substituted with 1 to 10 size-limited substituent groups. In embodiments, when R³ is substituted, it is substituted with 1 to 10 lower substituent groups. In embodiments, when R³ is substituted, it is substituted with 1 to 5 substituent groups. In embodiments, when R³ is substituted, it is substituted with 1 to 5 size-limited substituent groups. In embodiments, when R³ is substituted, it is substituted with 1 to 5 lower substituent groups. In embodiments, when R³ is substituted, it is substituted with a substituent group. In embodiments, when R³ is substituted, it is substituted with a size-limited substituent group. In embodiments, when R³ is substituted, it is substituted with a lower substituent group.

In embodiments, R³ is hydrogen, halogen, —CCl₃, —CBr₃, —CF₃, —CI₃, —CHCl₂, —CHBr₂,

“\*MERGEFORMAT\*MERGEFORMAT —CHF₂, —CHI₂, —CH₂Cl, —CH₂Br, —CH₂F, —CH₂I, —CN, —OH, —NH₂, —COOH, —CONH₂, —NO₂, —SH,

“\*MERGEFORMAT\*MERGEFORMAT —SO₃H, —SO₄H, —SO₂NH₂, —NHNH₂, —ONH₂, —NHC(O)NHNH₂, —NHC(O)NH₂, —NHSO₂H,

“\*MERGEFORMAT\*MERGEFORMAT —NHC(O)H, —NHC(O)OH, —NHOH, —OCCl₃, —OCF₃, —OCBr₃, —OCI₃, —OCHCl₂, —OCHBr₂,

“\*MERGEFORMAT\*MERGEFORMAT —OCHI₂, —OCHF₂, —OCH₂Cl, —OCH₂Br, —OCH₂I, —OCH₂F, —N₃, —SF₅, R^{3A}-substituted or unsubstituted alkyl (e.g., C₁-C₂₀, C₁₀-C₂₀, C₁-C₈, C₁-C₆, or C₁-C₄), R^{3A}-substituted or unsubstituted heteroalkyl (e.g., 2 to 20, 8 to 20, 2 to 10, 2 to 8, 2 to 6, or 2 to 4 membered), R^{3A}-substituted or unsubstituted cycloalkyl (e.g., C₃-C₈, C₃-C₆, or C₅-C₆), R^{3A}-substituted or unsubstituted heterocycloalkyl (e.g., 3 to 8, 3 to 6, or 5 to 6 membered), R^{3A}-substituted or unsubstituted aryl (e.g., C₆-C₁₀, C₁₀, or phenyl), or R^{3A}-substituted or unsubstituted heteroaryl (e.g., 5 to 10, 5 to 9, or 5 to 6 membered). In embodiments, R³ is not hydrogen. In embodiments, R³ is hydrogen, halogen, —CCl₃, —CBr₃, —CF₃, —CI₃, —CHCl₂, —CHBr₂, —CHF₂, —CHI₂, —CH₂Cl,

“\*MERGEFORMAT\*MERGEFORMAT —CH₂Br, —CH₂F, —CH₂I, —CN, —OH, —NH₂, —COOH, —CONH₂, —NO₂, —SH, —SO₃H, —SO₄H,

“\*MERGEFORMAT\*MERGEFORMAT —SO₂NH₂, —NHNH₂, —ONH₂, —NHC(O)NHNH₂, —NHC(O)NH₂, —NHSO₂H, —NHC(O)H,

“\*MERGEFORMAT\*MERGEFORMAT —NHC(O)OH, —NHOH, —OCCl₃, —OCF₃, —OCBr₃, —OCI₃, —OCHCl₂, —OCHBr₂, —OCHI₂, —OCHF₂,

“\*MERGEFORMAT\*MERGEFORMAT —OCH₂Cl, —OCH₂Br, —OCH₂I, —OCH₂F, —N₃, —SF₅, substituted or unsubstituted alkyl (e.g., C₁-C₂₀, C₁₀-C₂₀, C₁-C₈, C₁-C₆, or C₁-C₄), substituted or unsubstituted heteroalkyl (e.g., 2 to 20, 8 to 20, 2 to 10, 2 to 8, 2 to 6, or 2 to 4 membered), substituted or unsubstituted cycloalkyl (e.g., C₃-C₈, C₃-C₆, or C₅-C₆), substituted or unsubstituted heterocycloalkyl (e.g., 3 to 8, 3 to 6, or 5 to 6 membered), substituted or unsubstituted aryl (e.g., C₆-C₁₀, C₁₀, or phenyl), or substituted or unsubstituted heteroaryl (e.g., 5 to 10, 5 to 9, or 5 to 6 membered). R^{3A} is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F,

“\*MERGEFORMAT\*MERGEFORMAT —CH₂I, —CN, —OH, —NH₂, —COOH, —CONH₂, —NO₂, —SH, —SO₃H, —SO₄H, —SO₂NH₂, —NHNH₂,

“\*MERGEFORMAT\*MERGEFORMAT —ONH₂, —NHC(O)NHNH₂, —NHC(O)NH₂, —NHSO₂H, —NHC(O)H, —NHC(O)OH, —NHOH,

“\*MERGEFORMAT\*MERGEFORMAT —OCCl₃, —OCF₃, —OCBr₃, —OCI₃, —OCHCl₂, —OCHBr₂, —OCHI₂, —OCHF₂, —OCH₂Cl, —OCH₂Br,

“\*MERGEFORMAT\*MERGEFORMAT —OCH₂I, —OCH₂F, —N₃, —SF₅, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, R³ is hydrogen. In embodiments, R³ is not hydrogen. In embodiments, R³ is substituted or unsubstituted alkyl (e.g., C₁-C₂₀, C₁₀-C₂₀, C₁-C₈, C₁-C₆, or C₁-C₄). In embodiments, R³ is substituted or unsubstituted C₁-C₂₀ alkyl. In embodiments, R³ is substituted or unsubstituted C₁₀-C₂₀ alkyl. In embodiments, R³ is substituted or unsubstituted C₁-C₈ alkyl. In embodiments, R³ is substituted or unsubstituted C₁-C₆ alkyl. In embodiments, R³ is substituted or unsubstituted C₁-C₄ alkyl. In embodiments, R³ is substituted or unsubstituted heteroalkyl (e.g., 2 to 20, 8 to 20, 2 to 10, 2 to 8, 2 to 6, or 2 to 4 membered). In embodiments, R³ is substituted or unsubstituted 2 to 20 membered heteroalkyl. In embodiments, R³ is substituted or unsubstituted 8 to 20 membered heteroalkyl. In embodiments, R³ is substituted or unsubstituted 2 to 10 membered heteroalkyl. In embodiments, R³ is substituted or unsubstituted 2 to 8 membered heteroalkyl. In embodiments, R³ is substituted or unsubstituted 2 to 6 heteroalkyl. In embodiments, R³ is substituted or unsubstituted 2 to 4 membered heteroalkyl. In embodiments, R³ is substituted or unsubstituted cycloalkyl (e.g., C₃-C₈, C₃-C₆, or C₅-C₆). In embodiments, R³ is substituted or unsubstituted C₃-C₈ cycloalkyl. In embodiments, R³ is substituted or unsubstituted C₃-C₆ cycloalkyl. In embodiments, R³ is substituted or unsubstituted C₅-C₈ cycloalkyl. In embodiments, R³ is substituted or unsubstituted heterocycloalkyl (e.g., 3 to 8, 3 to 6, or 5 to 6 membered). In embodiments, R³ is substituted or unsubstituted 3 to 8 heterocycloalkyl. In embodiments, R³ is substituted or unsubstituted 3 to 6 heterocycloalkyl. In embodiments, R³ is substituted or unsubstituted 5 to 6 heterocycloalkyl. In embodiments, R³ is substituted or unsubstituted aryl (e.g., C₆-C₁₀, C₁₀, or phenyl). In embodiments, R³ is substituted or unsubstituted C₆ aryl. In embodiments, R³ is substituted or unsubstituted C₇ aryl. In embodiments, R³ is substituted or unsubstituted C₈ aryl. In embodiments, R³ is substituted or unsubstituted C₉ aryl. In embodiments, R³ is substituted or unsubstituted C₁₀ aryl. In embodiments, R³ is substituted or unsubstituted heteroaryl (e.g., 5 to 10, 5 to 9, or 5 to 6 membered). In embodiments, R³ is substituted or unsubstituted 5 to 10 membered heteroaryl. In embodiments, R³ is substituted or unsubstituted 5 to 9 membered heteroaryl. In embodiments, R³ is substituted or unsubstituted 5 to 6 membered heteroaryl. In embodiments, R³ is substituted or unsubstituted 5 membered heteroaryl. In embodiments, R³ is substituted or unsubstituted 6 membered heteroaryl. In embodiments, R³ is substituted or unsubstituted 7 membered heteroaryl. In embodiments, R³ is substituted or unsubstituted 8 membered heteroaryl. In embodiments, R³ is substituted or unsubstituted 9 membered heteroaryl. In embodiments, R³ is substituted or unsubstituted 10 membered heteroaryl.

In embodiments, R³ is hydrogen. In embodiments, R³ is —CH₃. In embodiments, R³ is unsubstituted C₁-C₆ alkyl. In embodiments, R³ is —F or —Cl. In embodiments, R³ is a halogen. In embodiments, R³ is —CN. In embodiments, R³ is phenyl.

In embodiments, R³ is substituted or unsubstituted C₁-C₆ alkyl. In embodiments, R³ is substituted or unsubstituted C₁-C₆ alkyl. In embodiments, R³ is substituted or unsubstituted C₁ alkyl. In embodiments, R³ is substituted or unsubstituted C₂ alkyl. In embodiments, R³ is substituted or unsubstituted C₃ alkyl. In embodiments, R³ is substituted or unsubstituted C₄ alkyl. In embodiments, R³ is substituted or unsubstituted C₅ alkyl. In embodiments, R³ is substituted or unsubstituted C₆ alkyl. In embodiments, R³ is a substituted C₁ alkyl. In embodiments, R³ is a substituted C₂ alkyl. In embodiments, R³ is a substituted C₃ alkyl. In embodiments, R³ is a substituted C₄ alkyl. In embodiments, R³ is a substituted C₅ alkyl. In embodiments, R³ is a substituted C₆ alkyl. In embodiments, R³ is an unsubstituted C₁ alkyl. In embodiments, R³ is an unsubstituted C₂ alkyl. In embodiments, R³ is an unsubstituted C₃ alkyl. In embodiments, R³ is an unsubstituted C₄ alkyl. In embodiments, R³ is an unsubstituted C₅ alkyl. In embodiments, R³ is an unsubstituted C₆ alkyl.

In embodiments, R³ is substituted or unsubstituted 2 to 8 membered heteroalkyl. In embodiments, R³ is substituted or unsubstituted 2 to 6 membered heteroalkyl. In embodiments, R³ is substituted or unsubstituted 2 to 4 membered heteroalkyl. In embodiments, R³ is a substituted 2 to 8 membered heteroalkyl. In embodiments, R³ is a substituted 2 to 6 membered heteroalkyl. In embodiments, R³ is a substituted 2 to 4 membered heteroalkyl. In embodiments, R³ is an unsubstituted 2 to 8 membered heteroalkyl. In embodiments, R³ is an unsubstituted 2 to 6 membered heteroalkyl. In embodiments, R³ is an unsubstituted 2 to 4 membered heteroalkyl.

In embodiments, R³ is substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R³ is substituted or unsubstituted cycloalkyl (e.g., C₃-C₈, C₃-C₆, C₄-C₆, or C₅-C₆). In embodiments, R³ is substituted cycloalkyl (e.g., C₃-C₈, C₃-C₆, C₄-C₆, or C₅-C₆). In embodiments, R³ is an unsubstituted cycloalkyl (e.g., C₃-C₈, C₃-C₆, C₄-C₆, or C₅-C₆). In embodiments, R³ is substituted or unsubstituted heterocycloalkyl (e.g., 3 to 8 membered, 3 to 6 membered, 4 to 6 membered, 4 to 5 membered, or 5 to 6 membered). In embodiments, R³ is substituted heterocycloalkyl (e.g., 3 to 8 membered, 3 to 6 membered, 4 to 6 membered, 4 to 5 membered, or 5 to 6 membered). In embodiments, R³ is an unsubstituted heterocycloalkyl (e.g., 3 to 8 membered, 3 to 6 membered, 4 to 6 membered, 4 to 5 membered, or 5 to 6 membered).

In embodiments, R³ is substituted or unsubstituted aryl (e.g., C₆-C₁₀, C₁₀, or phenyl). In embodiments, R³ is substituted aryl (e.g., C₆-C₁₀, C₁₀, or phenyl). In embodiments, R³ is unsubstituted aryl (e.g., C₆-C₁₀, C₁₀, or phenyl). In embodiments, R³ is unsubstituted phenyl. In embodiments, R³ is substituted or unsubstituted heteroaryl (e.g., 5 to 10, 5 to 9, or 5 to 6 membered). In embodiments, R³ is substituted heteroaryl (e.g., 5 to 10, 5 to 9, or 5 to 6 membered). In embodiments, R³ is unsubstituted heteroaryl (e.g., 5 to 10, 5 to 9, or 5 to 6 membered). In embodiments, R³ is a substituted or unsubstituted 5 membered heteroaryl. In embodiments, R³ is a substituted or unsubstituted 6 membered heteroaryl. In embodiments, R³ is a substituted or unsubstituted 5 membered heteroaryl. In embodiments, R³ is an unsubstituted 5 membered heteroaryl. In embodiments, R³ is an unsubstituted 6 membered heteroaryl. In embodiments, R³ is an unsubstituted 7 membered heteroaryl.

In embodiments, R³ is substituted or unsubstituted alkyl. In embodiments, R³ is substituted or unsubstituted aryl, or substituted or unsubstituted heteroaryl. In embodiments, R³ is substituted or unsubstituted aryl. In embodiments, R³ is,

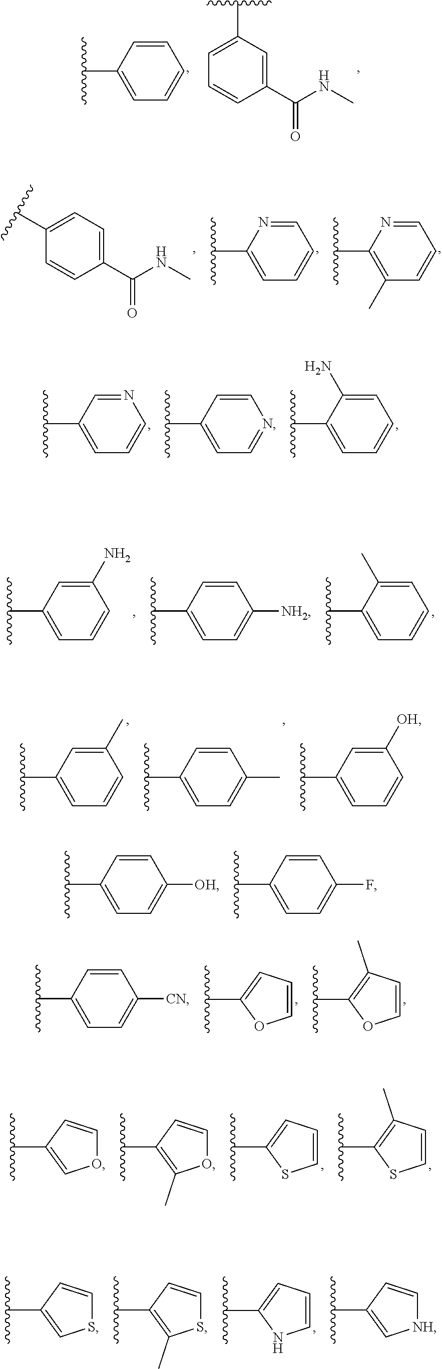

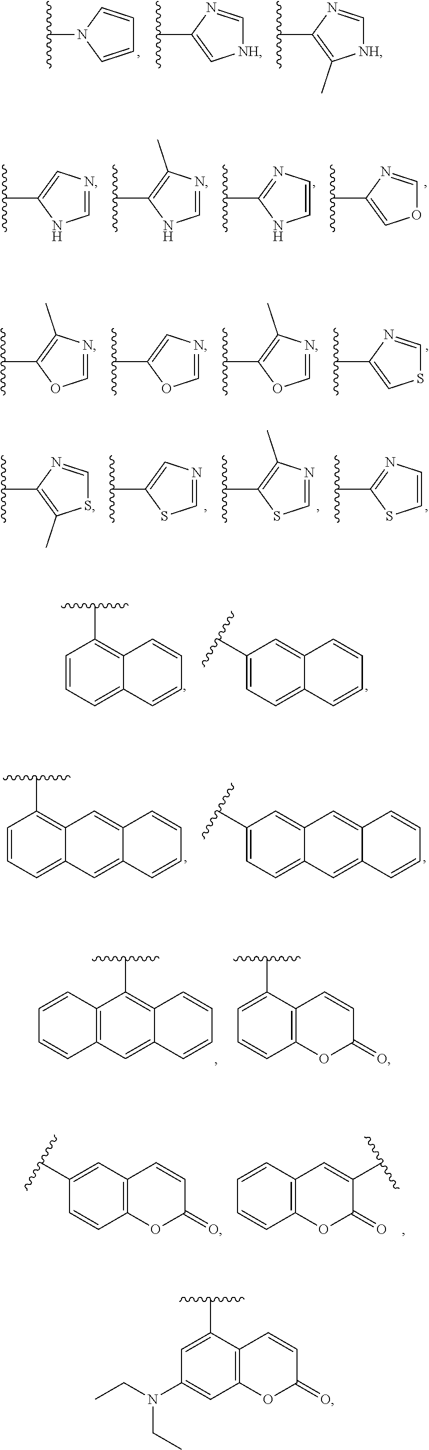

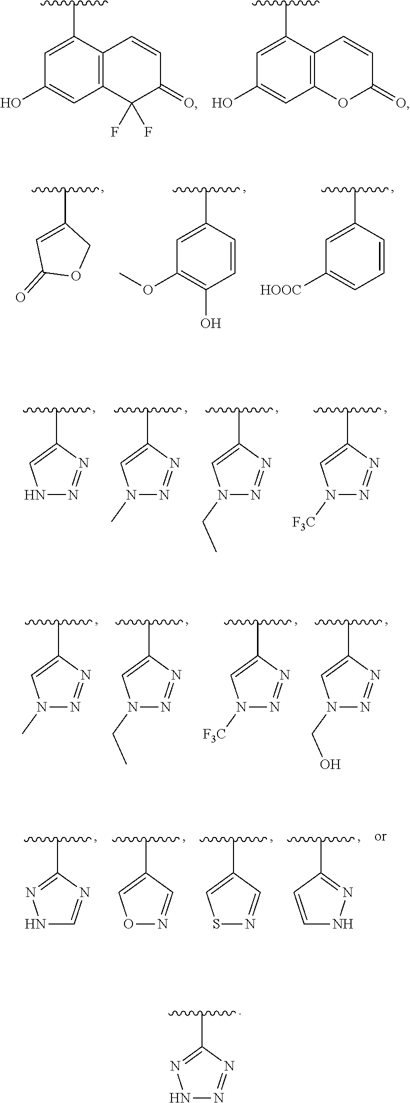

In embodiments, R³ is

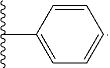 [SMILES: *c1ccccc1]

In embodiments, R³ is

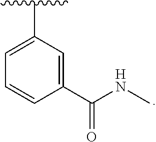 [SMILES: *c1cccc(C(=O)NC)c1]

In embodiments, R³ is

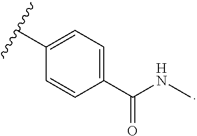 [SMILES: *c1ccc(C(=O)NC)cc1]

In embodiments, R³ is

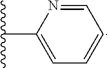 [SMILES: *c1ccccn1]

In embodiments, R³ is

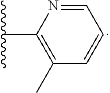 [SMILES: *c1ncccc1C]

In embodiments, R³ is

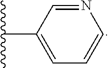 [SMILES: *c1cccnc1]

In embodiments, R³ is

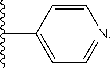 [SMILES: *c1ccncc1]

In embodiments, R³ is

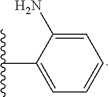 [SMILES: *c1ccccc1N]

In embodiments, R³ is

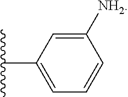 [SMILES: *c1cccc(N)c1]

In embodiments, R³ is

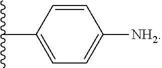 [SMILES: *c1ccc(N)cc1]

In embodiments, R³ is

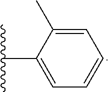 [SMILES: *c1ccccc1C]

In embodiments, R³ is

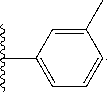 [SMILES: *c1cccc(C)c1]

In embodiments, R³ is

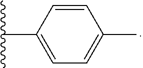 [SMILES: *c1ccc(C)cc1]

In embodiments, R³ is

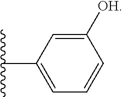 [SMILES: *c1cccc(O)c1]

In embodiments, R³ is

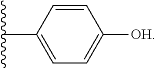 [SMILES: *c1ccc(O)cc1]

In embodiments, R³ is

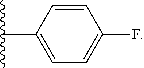 [SMILES: *c1ccc(F)cc1]

In embodiments, R³ is

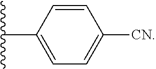 [SMILES: *c1ccc(C#N)cc1]

In embodiments, R³ is

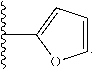 [SMILES: *c1ccco1]

In embodiments, R³ is

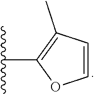 [SMILES: *c1occc1C]

In embodiments, R³ is

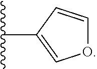 [SMILES: *c1ccoc1]

In embodiments, R³ is

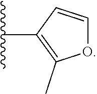 [SMILES: *c1ccoc1C]

In embodiments, R³ is

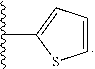 [SMILES: *c1cccs1]

In embodiments, R³ is

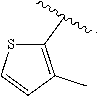 [SMILES: *c1sccc1C]

In embodiments, R³ is

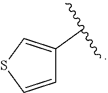 [SMILES: *c1ccsc1]

In embodiments, R³ is

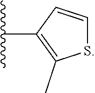 [SMILES: *c1ccsc1C]

In embodiments, R³ is

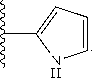 [SMILES: *c1ccc[nH]1]

In embodiments, R³ is

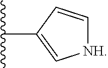 [SMILES: *c1cc[nH]c1]

In embodiments, R³ is

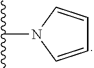 [SMILES: *n1cccc1]

In embodiments, R³ is

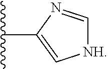 [SMILES: *c1c[nH]cn1]

In embodiments, R³ is

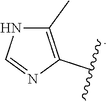 [SMILES: *c1nc[nH]c1C]

In embodiments, R³ is

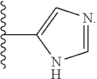 [SMILES: *c1cnc[nH]1]

In embodiments, R³ is

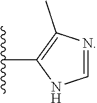 [SMILES: *c1[nH]cnc1C]

In embodiments, R³ is

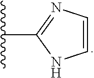 [SMILES: *c1ncc[nH]1]

In embodiments, R³ is

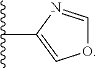 [SMILES: *c1cocn1]

In embodiments, R³ is

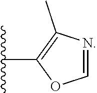 [SMILES: *c1ocnc1C]

In embodiments, R³ is

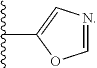 [SMILES: *c1cnco1]

In embodiments, R³ is

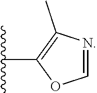 [SMILES: *c1ocnc1C]

In embodiments, R³ is

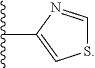 [SMILES: *c1cscn1]

In embodiments, R³ is

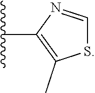 [SMILES: *c1ncsc1C]

In embodiments, R³ is

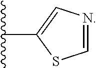 [SMILES: *c1cncs1]

In embodiments, R³ is

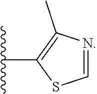 [SMILES: *c1scnc1C]

In embodiments, R³ is

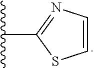 [SMILES: *c1nccs1]

In embodiments, R³ is

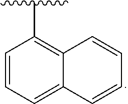 [SMILES: *c1cccc2ccccc12]

In embodiments, R³ is

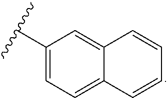 [SMILES: *c1ccc2ccccc2c1]

In embodiments, R³ is

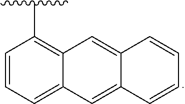 [SMILES: *c1cccc2cc3ccccc3cc12]

In embodiments, R³ is

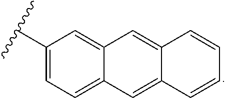 [SMILES: *c1ccc2cc3ccccc3cc2c1]

In embodiments, R³ is

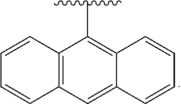 [SMILES: *c1c2ccccc2cc2ccccc12]

In embodiments, R³ is

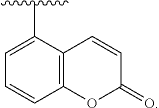 [SMILES: *c1cccc2oc(=O)ccc12]

In embodiments, R³ is

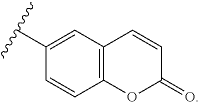 [SMILES: *c1ccc2oc(=O)ccc2c1]

In embodiments, R³ is

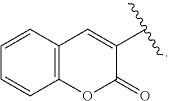 [SMILES: *c1cc2ccccc2oc1=O]

In embodiments, R³ is

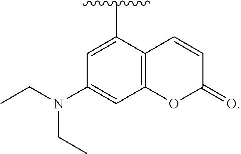 [SMILES: *c1cc(N(CC)CC)cc2oc(=O)ccc12]

In embodiments, R³ is

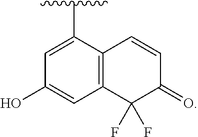 [SMILES: *c1cc(O)cc2c1C=CC(=O)C2(F)F]

In embodiments, R³ is

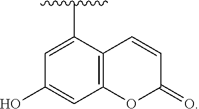 [SMILES: *c1cc(O)cc2oc(=O)ccc12]

In embodiments, R³ is

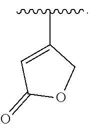 [SMILES: *C1=CC(=O)OC1]

In embodiments, R³ is

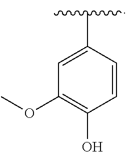 [SMILES: *c1ccc(O)c(OC)c1]

In embodiments, R³ is

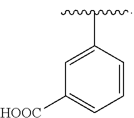 [SMILES: *c1cccc(C(=O)O)c1]

In embodiments, R³ is

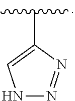 [SMILES: *c1c[nH]nn1]

In embodiments, R³ is

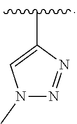 [SMILES: *c1cn(C)nn1]

In embodiments, R³ is

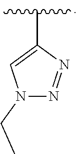 [SMILES: *c1cn(CC)nn1]

In embodiments, R³ is

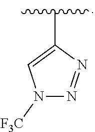 [SMILES: *c1cn(C(F)(F)F)nn1]

In embodiments, R³ is

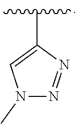 [SMILES: *c1cn(C)nn1]

In embodiments, R³ is

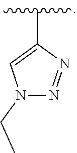 [SMILES: *c1cn(CC)nn1]

In embodiments, R³ is

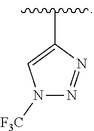 [SMILES: *c1cn(C(F)(F)F)nn1]

In embodiments, R³ is

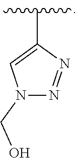 [SMILES: *c1cn(CO)nn1]

In embodiments, R³ is

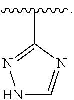 [SMILES: *c1nc[nH]n1]

In embodiments, R³ is

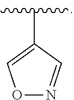 [SMILES: *c1cnoc1]

In embodiments, R³ is

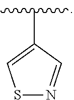 [SMILES: *c1cnsc1]

In embodiments, R³ is

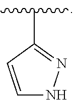 [SMILES: *c1cc[nH]n1]

In embodiments, R³ is

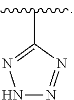 [SMILES: *c1nn[nH]n1]

In embodiments, R³ is halogen, —CCl₃, —CBr₃, —CF₃, —CI₃, —CHCl₂, —CHBr₂, —CHF₂,

“\*MERGEFORMAT\*MERGEFORMAT —CHI₂, —CH₂Cl, —CH₂Br, —CH₂F, —CH₂I, —CN, —OH, —NH₂, —COOH, —CONH₂, —NO₂, —SH, —SO₃H,

“\*MERGEFORMAT\*MERGEFORMAT —SO₄H, —SO₂NH₂, —NHNH₂, —ONH₂, —NHC(O)NHNH₂, —NHC(O)NH₂, —NHSO₂H, —NHC(O)H,

“\*MERGEFORMAT\*MERGEFORMAT —NHC(O)OH, —NHOH, —OCCl₃, —OCF₃, —OCBr₃, —OCI₃, —OCHCl₂, —OCHBr₂, —OCHI₂, —OCHF₂,

“\*MERGEFORMAT\*MERGEFORMAT —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is a substituted or unsubstituted alkyl. In embodiments, R³ is a substituted or unsubstituted aryl, or substituted or unsubstituted heteroaryl. In embodiments, R³ is a substituted or unsubstituted aryl.

In embodiments, R⁴ is a substituted (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group. In embodiments, when R⁴ is substituted, it is substituted with 1 to 10 substituent groups. In embodiments, when R⁴ is substituted, it is substituted with 1 to 10 size-limited substituent groups. In embodiments, when R⁴ is substituted, it is substituted with 1 to 10 lower substituent groups. In embodiments, when R⁴ is substituted, it is substituted with 1 to 5 substituent groups. In embodiments, when R⁴ is substituted, it is substituted with 1 to 5 size-limited substituent groups. In embodiments, when R⁴ is substituted, it is substituted with 1 to 5 lower substituent groups. In embodiments, when R⁴ is substituted, it is substituted with a substituent group. In embodiments, when R⁴ is substituted, it is substituted with a size-limited substituent group. In embodiments, when R⁴ is substituted, it is substituted with a lower substituent group.

In embodiments, R⁴ is hydrogen, halogen, —CCl₃, —CBr₃, —CF₃, —CI₃, —CHCl₂, —CHBr₂,

“\*MERGEFORMAT\*MERGEFORMAT —CHF₂, —CHI₂, —CH₂Cl, —CH₂Br, —CH₂F, —CH₂I, —CN, —OH, —NH₂, —COOH, —CONH₂, —NO₂, —SH,

“\*MERGEFORMAT\*MERGEFORMAT —SO₃H, —SO₄H, —SO₂NH₂, —NHNH₂, —ONH₂, —NHC(O)NHNH₂, —NHC(O)NH₂, —NHSO₂H,

“\*MERGEFORMAT\*MERGEFORMAT —NHC(O)H, —NHC(O)OH, —NHOH, —OCCl₃, —OCF₃, —OCBr₃, —OCI₃, —OCHCl₂, —OCHBr₂,

“\*MERGEFORMAT\*MERGEFORMAT —OCHI₂, —OCHF₂, —OCH₂Cl, —OCH₂Br, —OCH₂I, —OCH₂F, —N₃, —SF₅, R^{4A}-substituted or unsubstituted alkyl (e.g., C₁-C₂₀, C₁₀-C₂₀, C₁-C₈, C₁-C₆, or C₁-C₄), R^{4A}-substituted or unsubstituted heteroalkyl (e.g., 2 to 20, 8 to 20, 2 to 10, 2 to 8, 2 to 6, or 2 to 4 membered), R^{4A}-substituted or unsubstituted cycloalkyl (e.g., C₃-C₈, C₃-C₆, or C₅-C₆), R^{4A}-substituted or unsubstituted heterocycloalkyl (e.g., 3 to 8, 3 to 6, or 5 to 6 membered), R^{4A}-substituted or unsubstituted aryl (e.g., C₆-C₁₀, C₁₀, or phenyl), or R^{4A}-substituted or unsubstituted heteroaryl (e.g., 5 to 10, 5 to 9, or 5 to 6 membered). In embodiments, R⁴ is hydrogen, halogen, —CCl₃,

“*\MERGEFORMAT\*MERGEFORMAT —CBr₃, —CF₃, —CI₃, —CHCl₂, —CHBr₂, —CHF₂, —CHI₂, —CH₂Cl, —CH₂Br, —CH₂F, —CH₂I, —CN, —OH,

“*\MERGEFORMAT\*MERGEFORMAT —NH₂, —COOH, —CONH₂, —NO₂, —SH, —SO₃H, —SO₄H, —SO₂NH₂, —NHNH₂, —ONH₂,

“\*MERGEFORMAT\*MERGEFORMAT —NHC(O)NHNH₂, —NHC(O)NH₂, —NHSO₂H, —NHC(O)H, —NHC(O)OH, —NHOH, —OCCl₃,

“\*MERGEFORMAT\*MERGEFORMAT —OCF₃, —OCBr₃, —OCI₃, —OCHCl₂, —OCHBr₂, —OCHI₂, —OCHF₂, —OCH₂Cl, —OCH₂Br, —OCH₂I,

“\*MERGEFORMAT\*MERGEFORMAT —OCH₂F, —N₃, —SF₅, substituted or unsubstituted alkyl (e.g., C₁-C₂₀, C₁₀-C₂₀, C₁-C₈, C₁-C₆, or C₁-C₄), substituted or unsubstituted heteroalkyl (e.g., 2 to 20, 8 to 20, 2 to 10, 2 to 8, 2 to 6, or 2 to 4 membered), substituted or unsubstituted cycloalkyl (e.g., C₃-C₈, C₃-C₆, or C₅-C₆), substituted or unsubstituted heterocycloalkyl (e.g., 3 to 8, 3 to 6, or 5 to 6 membered), substituted or unsubstituted aryl (e.g., C₆-C₁₀, C₁₀, or phenyl), or substituted or unsubstituted heteroaryl (e.g., 5 to 10, 5 to 9, or 5 to 6 membered). R^{4A} is independently oxo, halogen, —CCl₃, —CBr₃, —CF₃, —CI₃,

“\*MERGEFORMAT\*MERGEFORMAT —CHCl₂, —CHBr₂, —CHF₂, —CHI₂, —CH₂Cl, —CH₂Br, —CH₂F, —CH₂I, —CN, —OH, —NH₂, —COOH,

“\*MERGEFORMAT\*MERGEFORMAT —CONH₂, —NO₂, —SH, —SO₃H, —SO₄H, —SO₂NH₂, —NHNH₂, —ONH₂, —NHC(O)NHNH₂,

“\*MERGEFORMAT\*MERGEFORMAT —NHC(O)NH₂, —NHSO₂H, —NHC(O)H, —NHC(O)OH, —NHOH, —OCCl₃, —OCF₃, —OCBr₃, —OCI₃,

“\*MERGEFORMAT\*MERGEFORMAT —OCHCl₂, —OCHBr₂, —OCHI₂, —OCHF₂, —OCH₂Cl, —OCH₂Br, —OCH₂I, —OCH₂F, —N₃, —SF₅, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, R⁴ is hydrogen. In embodiments, R⁴ is not hydrogen. In embodiments, R⁴ is substituted or unsubstituted alkyl (e.g., C₁-C₂₀, C₁₀-C₂₀, C₁-C₈, C₁-C₆, or C₁-C₄). In embodiments, R⁴ is substituted or unsubstituted C₁-C₂₀ alkyl. In embodiments, R⁴ is substituted or unsubstituted C₁₀-C₂₀ alkyl. In embodiments, R⁴ is substituted or unsubstituted C₁-C₈ alkyl. In embodiments, R⁴ is substituted or unsubstituted C₁-C₆ alkyl. In embodiments, R⁴ is substituted or unsubstituted C₁-C₄ alkyl. In embodiments, R⁴ is substituted or unsubstituted heteroalkyl (e.g., 2 to 20, 8 to 20, 2 to 10, 2 to 8, 2 to 6, or 2 to 4 membered). In embodiments, R⁴ is substituted or unsubstituted 2 to 20 membered heteroalkyl. In embodiments, R⁴ is substituted or unsubstituted 8 to 20 membered heteroalkyl. In embodiments, R⁴ is substituted or unsubstituted 2 to 10 membered heteroalkyl. In embodiments, R⁴ is substituted or unsubstituted 2 to 8 membered heteroalkyl. In embodiments, R⁴ is substituted or unsubstituted 2 to 6 heteroalkyl. In embodiments, R⁴ is substituted or unsubstituted 2 to 4 membered heteroalkyl. In embodiments, R⁴ is substituted or unsubstituted cycloalkyl (e.g., C₃-C₈, C₃-C₆, or C₅-C₆). In embodiments, R⁴ is substituted or unsubstituted C₃-C₈ cycloalkyl. In embodiments, R⁴ is substituted or unsubstituted C₃-C₆ cycloalkyl. In embodiments, R⁴ is substituted or unsubstituted C₅-C₈ cycloalkyl. In embodiments, R⁴ is substituted or unsubstituted heterocycloalkyl (e.g., 3 to 8, 3 to 6, or 5 to 6 membered). In embodiments, R⁴ is substituted or unsubstituted 3 to 8 heterocycloalkyl. In embodiments, R⁴ is substituted or unsubstituted 3 to 6 heterocycloalkyl. In embodiments, R⁴ is substituted or unsubstituted 5 to 6 heterocycloalkyl. In embodiments, R⁴ is substituted or unsubstituted aryl (e.g., C₆-C₁₀, C₁₀, or phenyl). In embodiments, R⁴ is substituted or unsubstituted C₆ aryl. In embodiments, R⁴ is substituted or unsubstituted C₇ aryl. In embodiments, R⁴ is substituted or unsubstituted C₈ aryl. In embodiments, R⁴ is substituted or unsubstituted C₉ aryl. In embodiments, R⁴ is substituted or unsubstituted C₁₀ aryl. In embodiments, R⁴ is substituted or unsubstituted heteroaryl (e.g., 5 to 10, 5 to 9, or 5 to 6 membered). In embodiments, R⁴ is substituted or unsubstituted 5 to 10 membered heteroaryl. In embodiments, R⁴ is substituted or unsubstituted 5 to 9 membered heteroaryl. In embodiments, R⁴ is substituted or unsubstituted 5 to 6 membered heteroaryl. In embodiments, R⁴ is substituted or unsubstituted 5 membered heteroaryl. In embodiments, R⁴ is substituted or unsubstituted 6 membered heteroaryl. In embodiments, R⁴ is substituted or unsubstituted 7 membered heteroaryl. In embodiments, R⁴ is substituted or unsubstituted 8 membered heteroaryl. In embodiments, R⁴ is substituted or unsubstituted 9 membered heteroaryl. In embodiments, R⁴ is substituted or unsubstituted 10 membered heteroaryl.

In embodiments, R⁴ is hydrogen. In embodiments, R⁴ is —CH₃. In embodiments, R⁴ is unsubstituted C₁-C₆ alkyl. In embodiments, R⁴ is —F or —Cl. In embodiments, R⁴ is a halogen. In embodiments, R⁴ is —CN. In embodiments, R⁴ is phenyl.

In embodiments, R⁴ is substituted or unsubstituted C₁-C₆ alkyl. In embodiments, R⁴ is substituted or unsubstituted C₁-C₆ alkyl. In embodiments, R⁴ is substituted or unsubstituted C₁ alkyl. In embodiments, R⁴ is substituted or unsubstituted C₂ alkyl. In embodiments, R⁴ is substituted or unsubstituted C₃ alkyl. In embodiments, R⁴ is substituted or unsubstituted C₄ alkyl. In embodiments, R⁴ is substituted or unsubstituted C₅ alkyl. In embodiments, R⁴ is substituted or unsubstituted C₆ alkyl. In embodiments, R⁴ is a substituted C₁ alkyl. In embodiments, R⁴ is a substituted C₂ alkyl. In embodiments, R⁴ is a substituted C₃ alkyl. In embodiments, R⁴ is a substituted C₄ alkyl. In embodiments, R⁴ is a substituted C₅ alkyl. In embodiments, R⁴ is a substituted C₆ alkyl. In embodiments, R⁴ is an unsubstituted C₁ alkyl. In embodiments, R⁴ is an unsubstituted C₂ alkyl. In embodiments, R⁴ is an unsubstituted C₃ alkyl. In embodiments, R⁴ is an unsubstituted C₄ alkyl. In embodiments, R⁴ is an unsubstituted C₅ alkyl. In embodiments, R⁴ is an unsubstituted C₆ alkyl.

In embodiments, R⁴ is substituted or unsubstituted 2 to 8 membered heteroalkyl. In embodiments, R⁴ is substituted or unsubstituted 2 to 6 membered heteroalkyl. In embodiments, R⁴ is substituted or unsubstituted 2 to 4 membered heteroalkyl. In embodiments, R⁴ is a substituted 2 to 8 membered heteroalkyl. In embodiments, R⁴ is a substituted 2 to 6 membered heteroalkyl. In embodiments, R⁴ is a substituted 2 to 4 membered heteroalkyl. In embodiments, R⁴ is an unsubstituted 2 to 8 membered heteroalkyl. In embodiments, R⁴ is an unsubstituted 2 to 6 membered heteroalkyl. In embodiments, R⁴ is an unsubstituted 2 to 4 membered heteroalkyl.

In embodiments, R⁴ is substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁴ is substituted or unsubstituted cycloalkyl (e.g., C₃-C₈, C₃-C₆, C₄-C₆, or C₅-C₆). In embodiments, R⁴ is substituted cycloalkyl (e.g., C₃-C₈, C₃-C₆, C₄-C₆, or C₅-C₆). In embodiments, R⁴ is an unsubstituted cycloalkyl (e.g., C₃-C₈, C₃-C₆, C₄-C₆, or C₅-C₆). In embodiments, R⁴ is substituted or unsubstituted heterocycloalkyl (e.g., 3 to 8 membered, 3 to 6 membered, 4 to 6 membered, 4 to 5 membered, or 5 to 6 membered). In embodiments, R⁴ is substituted heterocycloalkyl (e.g., 3 to 8 membered, 3 to 6 membered, 4 to 6 membered, 4 to 5 membered, or 5 to 6 membered). In embodiments, R⁴ is an unsubstituted heterocycloalkyl (e.g., 3 to 8 membered, 3 to 6 membered, 4 to 6 membered, 4 to 5 membered, or 5 to 6 membered).

In embodiments, R⁴ is substituted or unsubstituted aryl (e.g., C₆-C₁₀, C₁₀, or phenyl). In embodiments, R⁴ is substituted aryl (e.g., C₆-C₁₀, C₁₀, or phenyl). In embodiments, R⁴ is unsubstituted aryl (e.g., C₆-C₁₀, C₁₀, or phenyl). In embodiments, R⁴ is unsubstituted phenyl. In embodiments, R⁴ is substituted or unsubstituted heteroaryl (e.g., 5 to 10, 5 to 9, or 5 to 6 membered). In embodiments, R⁴ is substituted heteroaryl (e.g., 5 to 10, 5 to 9, or 5 to 6 membered). In embodiments, R⁴ is unsubstituted heteroaryl (e.g., 5 to 10, 5 to 9, or 5 to 6 membered). In embodiments, R⁴ is a substituted or unsubstituted 5 membered heteroaryl. In embodiments, R⁴ is a substituted or unsubstituted 6 membered heteroaryl. In embodiments, R⁴ is a substituted or unsubstituted 5 membered heteroaryl. In embodiments, R⁴ is an unsubstituted 5 membered heteroaryl. In embodiments, R⁴ is an unsubstituted 6 membered heteroaryl. In embodiments, R⁴ is an unsubstituted 7 membered heteroaryl.

In embodiments, R⁴ is substituted or unsubstituted alkyl. In embodiments, R⁴ is substituted or unsubstituted aryl, or substituted or unsubstituted heteroaryl. In embodiments, R⁴ is substituted or unsubstituted aryl. In embodiments, R⁴ is

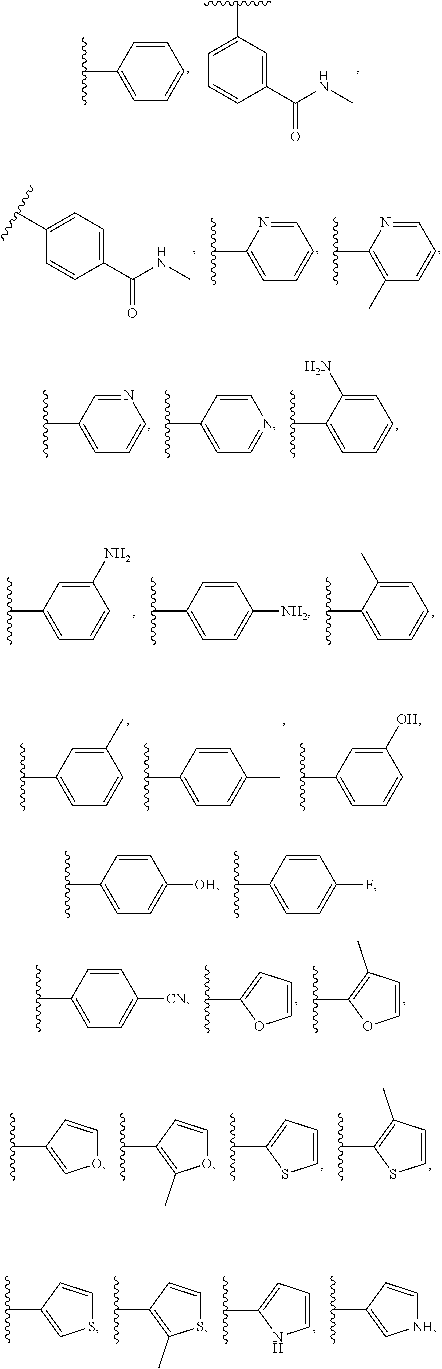

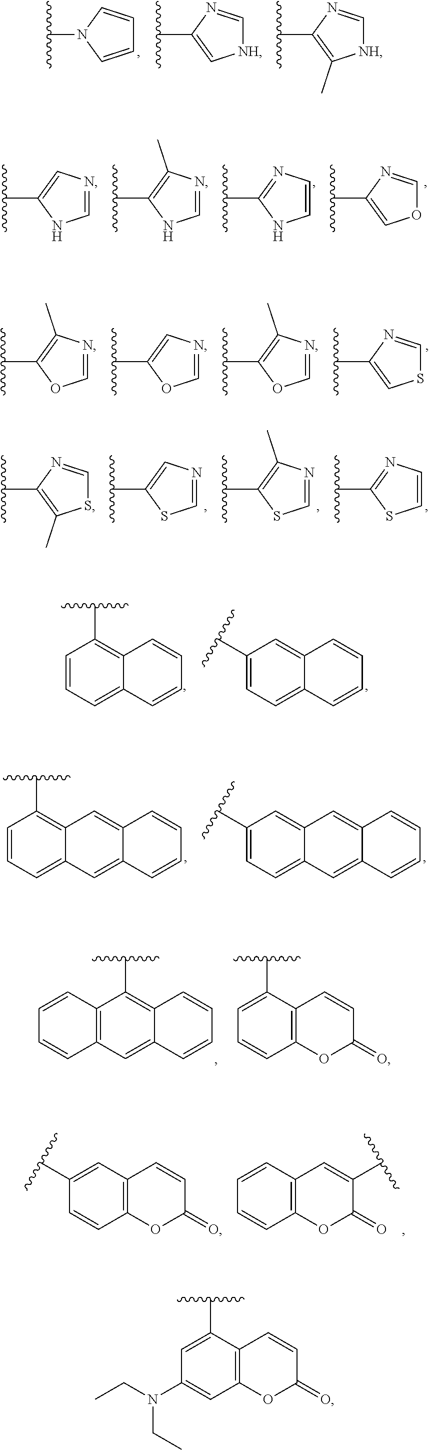

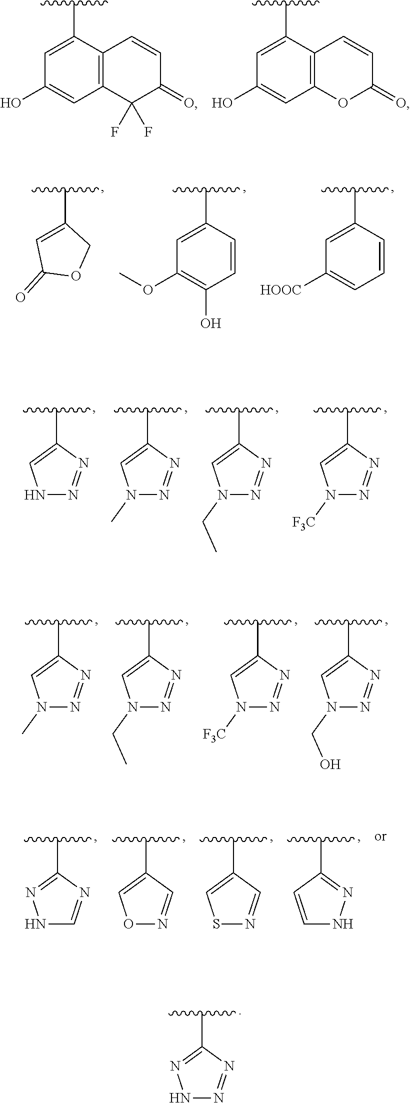

In embodiments, R⁴ is

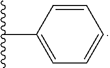 [SMILES: *c1ccccc1]

In embodiments, R⁴ is

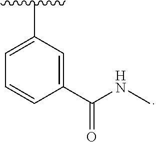 [SMILES: *c1cccc(C(=O)NC)c1]

In embodiments, R⁴ is

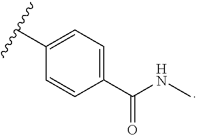 [SMILES: *c1ccc(C(=O)NC)cc1]

In embodiments, R⁴ is

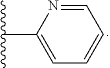 [SMILES: *c1ccccn1]

In embodiments, R⁴ is

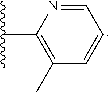 [SMILES: *c1ncccc1C]

In embodiments, R⁴ is

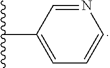 [SMILES: *c1cccnc1]

In embodiments, R⁴ is

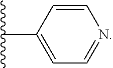 [SMILES: *c1ccncc1]

In embodiments, R⁴ is

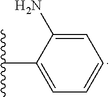 [SMILES: *c1ccccc1N]

In embodiments, R⁴ is

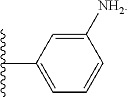 [SMILES: *c1cccc(N)c1]

In embodiments, R⁴ is

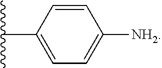 [SMILES: *c1ccc(N)cc1]

In embodiments, R⁴ is

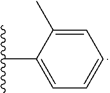 [SMILES: *c1ccccc1C]

In embodiments, R⁴ is

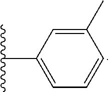 [SMILES: *c1cccc(C)c1]

In embodiments, R⁴ is

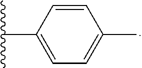 [SMILES: *c1ccc(C)cc1]

In embodiments, R⁴ is

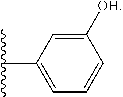 [SMILES: *c1cccc(O)c1]

In embodiments, R⁴ is

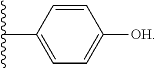 [SMILES: *c1ccc(O)cc1]

In embodiments, R⁴ is

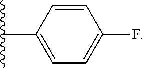 [SMILES: *c1ccc(F)cc1]

In embodiments, R⁴ is

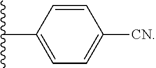 [SMILES: *c1ccc(C#N)cc1]

In embodiments, R⁴ is

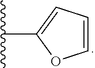 [SMILES: *c1ccco1]

In embodiments, R⁴ is

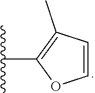 [SMILES: *c1occc1C]

In embodiments, R⁴ is

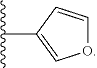 [SMILES: *c1ccoc1]

In embodiments, R⁴ is

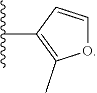 [SMILES: *c1ccoc1C]

In embodiments, R⁴ is

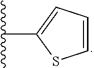 [SMILES: *c1cccs1]

In embodiments, R⁴ is

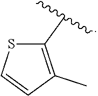 [SMILES: *c1sccc1C]

In embodiments, R⁴ is

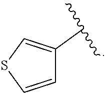 [SMILES: *c1ccsc1]

In embodiments, R⁴ is

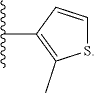 [SMILES: *c1ccsc1C]

In embodiments, R⁴ is

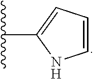 [SMILES: *c1ccc[nH]1]

In embodiments, R⁴ is

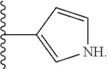 [SMILES: *c1cc[nH]c1]

In embodiments, R⁴ is

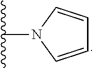 [SMILES: *n1cccc1]

In embodiments, R⁴ is

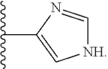 [SMILES: *c1c[nH]cn1]

In embodiments, R⁴ is

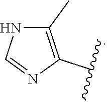 [SMILES: *c1nc[nH]c1C]

In embodiments, R⁴ is

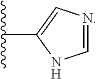 [SMILES: *c1cnc[nH]1]

In embodiments, R⁴ is

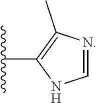 [SMILES: *c1[nH]cnc1C]

In embodiments, R⁴ is

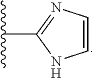 [SMILES: *c1ncc[nH]1]

In embodiments, R⁴ is

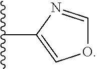 [SMILES: *c1cocn1]

In embodiments, R⁴ is

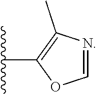 [SMILES: *c1ocnc1C]

In embodiments, R⁴ is

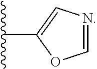 [SMILES: *c1cnco1]

In embodiments, R⁴ is

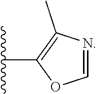 [SMILES: *c1ocnc1C]

In embodiments, R⁴ is

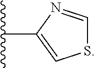 [SMILES: *c1cscn1]

In embodiments, R⁴ is

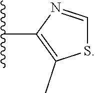 [SMILES: *c1ncsc1C]

In embodiments, R⁴ is

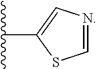 [SMILES: *c1cncs1]

In embodiments, R⁴ is

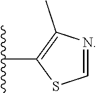 [SMILES: *c1scnc1C]

In embodiments, R⁴ is

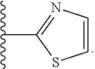 [SMILES: *c1nccs1]

In embodiments, R⁴ is

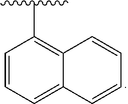 [SMILES: *c1cccc2ccccc12]

In embodiments, R⁴ is

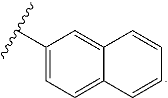 [SMILES: *c1ccc2ccccc2c1]

In embodiments, R⁴ is

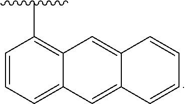 [SMILES: *c1cccc2cc3ccccc3cc12]

In embodiments, R⁴ is

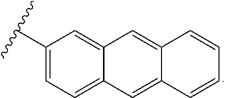 [SMILES: *c1ccc2cc3ccccc3cc2c1]

In embodiments, R⁴ is

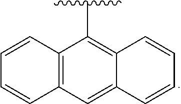 [SMILES: *c1c2ccccc2cc2ccccc12]

In embodiments, R⁴ is

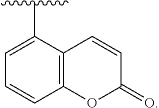 [SMILES: *c1cccc2oc(=O)ccc12]

In embodiments, R⁴ is

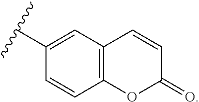 [SMILES: *c1ccc2oc(=O)ccc2c1]

In embodiments, R⁴ is

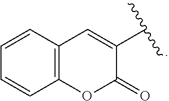 [SMILES: *c1cc2ccccc2oc1=O]

In embodiments, R⁴ is

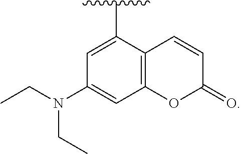 [SMILES: *c1cc(N(CC)CC)cc2oc(=O)ccc12]

In embodiments, R⁴ is

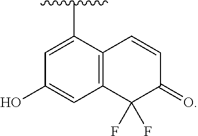 [SMILES: *c1cc(O)cc2c1C=CC(=O)C2(F)F]

In embodiments, R⁴ is

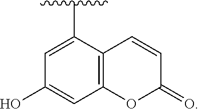 [SMILES: *c1cc(O)cc2oc(=O)ccc12]

In embodiments, R⁴ is

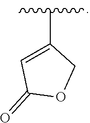 [SMILES: *C1=CC(=O)OC1]

In embodiments, R⁴ is

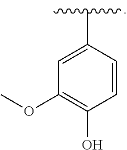 [SMILES: *c1ccc(O)c(OC)c1]

In embodiments, R⁴ is

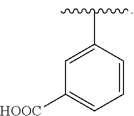 [SMILES: *c1cccc(C(=O)O)c1]

In embodiments, R⁴ is

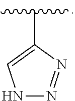 [SMILES: *c1c[nH]nn1]

In embodiments, R⁴ is

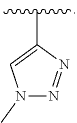 [SMILES: *c1cn(C)nn1]

In embodiments, R⁴ is

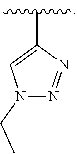 [SMILES: *c1cn(CC)nn1]

In embodiments, R⁴ is

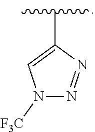 [SMILES: *c1cn(C(F)(F)F)nn1]

In embodiments, R⁴ is

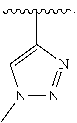 [SMILES: *c1cn(C)nn1]

In embodiments, R⁴ is

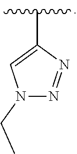 [SMILES: *c1cn(CC)nn1]

In embodiments, R⁴ is

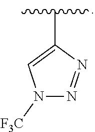 [SMILES: *c1cn(C(F)(F)F)nn1]

In embodiments, R⁴ is

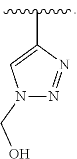 [SMILES: *c1cn(CO)nn1]

In embodiments, R⁴ is

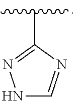 [SMILES: *c1nc[nH]n1]

In embodiments, R⁴ is

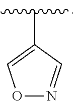 [SMILES: *c1cnoc1]

In embodiments, R⁴ is

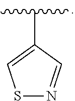 [SMILES: *c1cnsc1]

In embodiments, R⁴ is

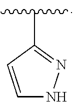 [SMILES: *c1cc[nH]n1]

In embodiments, R⁴ is

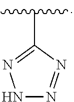 [SMILES: *c1nn[nH]n1]

In embodiments, R⁴ is hydrogen, halogen, —CCl₃, —CBr₃, —CF₃, —CI₃, —CHCl₂, —CHBr₂,

“\*MERGEFORMAT\*MERGEFORMAT —CHF₂, —CHI₂, —CH₂Cl, —CH₂Br, —CH₂F, —CH₂I, —CN, —OH, —NH₂, —COOH, —CONH₂, —NO₂, —SH,

“\*MERGEFORMAT\*MERGEFORMAT —SO₃H, —SO₄H, —SO₂NH₂, —NHNH₂, —ONH₂, —NHC(O)NHNH₂, —NHC(O)NH₂, —NHSO₂H,

“\*MERGEFORMAT\*MERGEFORMAT —NHC(O)H, —NHC(O)OH, —NHOH, —OCCl₃, —OCF₃, —OCBr₃, —OCI₃, —OCH Cl₂, —OCHBr₂,

“\*MERGEFORMAT\*MERGEFORMAT —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl groups.

In embodiments, R⁴ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is a substituted or unsubstituted alkyl. In embodiments, R⁴ is a substituted or unsubstituted aryl, or substituted or unsubstituted heteroaryl. In embodiments, R⁴ is a substituted or unsubstituted aryl.

In embodiments, L¹ and L² are independently covalent linkers.

In embodiments, L¹ is a bond, —NH, —S—, —O—, —C(O)—, —C(O)O—, —OC(O)—, —NHC(O)—,

“\*MERGEFORMAT\*MERGEFORMAT —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹ is not a bond. In embodiments, L¹ is a bond.

In embodiments, L¹ is -L^{1A}-L^{1B}-L^{1C}-, L^{1A}, L^{1B}, and L^{1C} are independently a bond, —NH—, —S—, —O—, —C(O)—, —C(O)O—, —OC(O)—, —NHC(O)—, —C(O)NH—, —NHC(O)NH—,

“\*MERGEFORMAT\*MERGEFORMAT —NHC(NH)NH—, —C(S)—, —N═N—, substituted or unsubstituted alkylene (e.g., —CH(OH)— or

“\*MERGEFORMAT\*MERGEFORMAT —C(CH₂)—), substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{1A}, L^{1B}, and L^{1C} are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{1A} is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L^{1A} is unsubstituted alkylene, unsubstituted heteroalkylene, unsubstituted cycloalkylene, unsubstituted heterocycloalkylene, unsubstituted arylene, or unsubstituted heteroarylene. In embodiments, L^{1B} is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L^{1B} is unsubstituted alkylene, unsubstituted heteroalkylene, unsubstituted cycloalkylene, unsubstituted heterocycloalkylene, unsubstituted arylene, or unsubstituted heteroarylene. In embodiments, L^{1C} is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L^{1C} is unsubstituted alkylene, unsubstituted heteroalkylene, unsubstituted cycloalkylene, unsubstituted heterocycloalkylene, unsubstituted arylene, or unsubstituted heteroarylene. In embodiments, L^{1A}, L^{1B}, and L^{1C} independently include PEG.

In embodiments, L¹ is R^{1A}-substituted or unsubstituted alkylene, R^{1A}-substituted or unsubstituted heteroalkylene, R^{1A}-substituted or unsubstituted cycloalkylene, R^{1A}-substituted or unsubstituted heterocycloalkylene, R^{1A}-substituted or unsubstituted arylene, or R^{1A}-substituted or unsubstituted heteroarylene. In embodiments, L¹ is R^{1A}-substituted or unsubstituted alkylene, or R^{1A}-substituted or unsubstituted heteroalkylene. In embodiments, L¹ is unsubstituted alkylene, or unsubstituted heteroalkylene.

R^{1A} is independently oxo, halogen, —CCl₃, —CBr₃, —CF₃, —CI₃, —CHCl₂, —CHBr₂, —CHF₂,

“\*MERGEFORMAT\*MERGEFORMAT —CHI₂, —CH₂Cl, —CH₂Br, —CH₂F, —CH₂I, —CN, —OH, —NH₂, —COOH, —CONH₂, —NO₂, —SH, —SO₃H,

“\*MERGEFORMAT\*MERGEFORMAT —SO₄H, —SO₂NH₂, —NHNH₂, —ONH₂, —NHC(O)NHNH₂, —NHC(O)NH₂, —NHSO₂H, —NHC(O)H,

“\*MERGEFORMAT\*MERGEFORMAT —NHC(O)OH, —NHOH, —OCCl₃, —OCF₃, —OCBr₃, —OCI₃, —OCHCl₂, —OCHBr₂, —OCHI₂, —OCHF₂,

“\*MERGEFORMAT\*MERGEFORMAT —OCH₂Cl, —OCH₂Br, —OCH₂I, —OCH₂F, —N₃, —SF₅, —NH₃⁺, —SO₃, —OPO₃H, —SCN, —ONO₂, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, L¹ is unsubstituted C₁-C₆ or C₁-C₄ alkylene. In embodiments, L¹ is unsubstituted C₁-C₄ alkylene. In embodiments, L¹ is unsubstituted C₁-C₆ alkylene. In embodiments, L¹ is unsubstituted methylene. In embodiments, L¹ is unsubstituted C₂ alkylene. In embodiments, L¹ is unsubstituted C₃ alkylene. In embodiments, L¹ is unsubstituted C₄ alkylene. In embodiments, L¹ is unsubstituted C₅ alkylene. In embodiments, L¹ is unsubstituted C₆ alkylene. In embodiments, L¹ is R^{1A}-substituted C₁-C₆ or C₁-C₄ alkylene. In embodiments, L¹ is R^{1A}-substituted C₁-C₄ alkylene. In embodiments, L¹ is R^{1A}-substituted C₁-C₆ alkylene. In embodiments, L¹ is R^{1A}-substituted methylene. In embodiments, L¹ is R^{1A}-substituted C₂ alkylene. In embodiments, L¹ is R^{1A}-substituted C₃ alkylene. In embodiments, L¹ is R^{1A}-substituted C₄ alkylene. In embodiments, L¹ is R^{1A}-substituted C₅ alkylene. In embodiments, L¹ is R^{1A}-substituted C₆ alkylene. In embodiments, L¹ is R^{1A}-substituted 2 to 10 membered heteroalkylene. In embodiments, L¹ is R^{1A}-substituted 2 to 8 membered heteroalkylene. In embodiments, L¹ is R^{1A}-substituted 2 to 6 membered heteroalkylene. In embodiments, L¹ is R^{1A}-substituted 2 to 4 membered heteroalkylene. In embodiments, L¹ is an unsubstituted 2 to 10 membered heteroalkylene. In embodiments, L¹ is an unsubstituted 2 to 8 membered heteroalkylene. In embodiments, L¹ is an unsubstituted 2 to 6 membered heteroalkylene. In embodiments, L¹ is an unsubstituted 2 to 4 membered heteroalkylene. In embodiments, L¹ is unsubstituted C₂-C₆ alkenylene or C₂-C₄ alkenylene. In embodiments, L¹ is unsubstituted C₂ alkenylene. In embodiments, L¹ is unsubstituted C₃ alkenylene. In embodiments, L¹ is unsubstituted C₄ alkenylene. In embodiments, L¹ is unsubstituted C₅ alkenylene. In embodiments, L¹ is unsubstituted C₆ alkenylene. In embodiments, L¹ is R^{1A}-substituted C₂-C₆ or C₂-C₄ alkenylene. In embodiments, L¹ is R^{1A}-substituted C₂-C₄ alkenylene. In embodiments, L¹ is R^{1A}-substituted C₂-C₆ alkenylene. In embodiments, L¹ is R^{1A}-substituted C₂ alkenylene. In embodiments, L¹ is R^{1A}-substituted C₃ alkenylene. In embodiments, L¹ is R^{1A}-substituted C₄ alkenylene. In embodiments, L¹ is R^{1A}-substituted C₅ alkenylene. In embodiments, L¹ is R^{1A}-substituted C₆ alkenylene. In embodiments, L¹ is R^{1A}-substituted 2 to 10 membered heteroalkenylene. In embodiments, L¹ is R^{1A}-substituted 2 to 8 membered heteroalkenylene. In embodiments, L¹ is R^{1A}-substituted 2 to 6 membered heteroalkenylene. In embodiments, L¹ is R^{1A}-substituted 2 to 4 membered heteroalkenylene. In embodiments, L¹ is an unsubstituted 2 to 10 membered heteroalkenylene. In embodiments, L¹ is an unsubstituted 2 to 8 membered heteroalkenylene. In embodiments, L¹ is an unsubstituted 2 to 6 membered heteroalkenylene. In embodiments, L¹ is an unsubstituted 2 to 4 membered heteroalkenylene.

In embodiments, L¹ is R^{1A}-substituted or unsubstituted alkylene (e.g., C₁-C₂₀, C₁₀-C₂₀, C₁-C₈, C₁-C₆, or C₁-C₄), or R^{1A}-substituted or unsubstituted heteroalkylene (e.g., 2 to 20, 8 to 20, 2 to 10, 2 to 8, 2 to 6, or 2 to 4 membered). In embodiments, L¹ is substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20, 8 to 20, 2 to 10, 2 to 8, 2 to 6, or 2 to 4 membered), or substituted or unsubstituted alkenylene (e.g., C₂-C₆ alkenylene or C₂-C₄ alkenylene).

In embodiments, L² is a bond, —NH, —S—, —O—, —C(O)—, —C(O)O—, —OC(O)—, —NHC(O)—,

“\*MERGEFORMAT\*MERGEFORMAT —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L² is a bond.

In embodiments, L² is -L^{2A}-L^{2B}-L^{2C}-. L^{2A}, L^{2B}, and L²c are independently a bond, —NH—, —S—, —O—, —C(O)—, —C(O)O—, —OC(O)—, —NHC(O)—, —C(O)NH—, —NHC(O)NH—,

“\*MERGEFORMAT\*MERGEFORMAT —NHC(NH)NH—, —C(S)—, —N═N—, substituted or unsubstituted alkylene (e.g., —CH(OH)— or

“\*MERGEFORMAT\*MERGEFORMAT —C(CH₂)—), substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{2A}, L^{2B}, and L^{2C} independently includes PEG. In embodiments, L^{2A}, L^{2B}, and L^{2C} are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{2A} is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L^{2A} is unsubstituted alkylene, unsubstituted heteroalkylene, unsubstituted cycloalkylene, unsubstituted heterocycloalkylene, unsubstituted arylene, or unsubstituted heteroarylene. In embodiments, L^{2B} is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L^{2B} is unsubstituted alkylene, unsubstituted heteroalkylene, unsubstituted cycloalkylene, unsubstituted heterocycloalkylene, unsubstituted arylene, or unsubstituted heteroarylene. In embodiments, L^{2C} is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L^{2C} is unsubstituted alkylene, unsubstituted heteroalkylene, unsubstituted cycloalkylene, unsubstituted heterocycloalkylene, unsubstituted arylene, or unsubstituted heteroarylene.

In embodiments, L² is R^{2A}-substituted or unsubstituted alkylene, R^{2A}-substituted or unsubstituted heteroalkylene, R^{2A}-substituted or unsubstituted cycloalkylene, R^{2A}-substituted or unsubstituted heterocycloalkylene, R^{2A} substituted or unsubstituted arylene, or R^{2A}-substituted or unsubstituted heteroarylene. In embodiments, L² is R^{2A}-substituted or unsubstituted alkylene, or R^{2A}-substituted or unsubstituted heteroalkylene. In embodiments, L² is unsubstituted alkylene, or unsubstituted heteroalkylene.

R^{2A} is independently oxo, halogen, —CCl₃, —CBr₃, —CF₃, —CI₃, —CHCl₂, —CHBr₂, —CHF₂,

“\*MERGEFORMAT*\MERGEFORMAT —CHI₂, —CH₂Cl, —CH₂Br, —CH₂F, —CH₂I, —CN, —OH, —NH₂, —COOH, —CONH₂, —NO₂, —SH, —SO₃H,

“\*MERGEFORMAT\*MERGEFORMAT —SO₄H, —SO₂NH₂, —NHNH₂, —ONH₂, —NHC(O)NHNH₂, —NHC(O)NH₂, —NHSO₂H, —NHC(O)H,

“\*MERGEFORMAT\*MERGEFORMAT —NHC(O)OH, —NHOH, —OCCl₃, —OCF₃, —OCBr₃, —OCI₃, —OCHCl₂, —OCHBr₂, —OCHI₂, —OCHF₂,

“\*MERGEFORMAT\*MERGEFORMAT —OCH₂Cl, —OCH₂Br, —OCH₂I, —OCH₂F, —N₃, —SF₅, —NH₃⁺, —SO₃, —OPO₃H, —SCN, —ONO₂, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, L² is unsubstituted C₁-C₆ or C₁-C₄ alkylene. In embodiments, L² is unsubstituted C₁-C₄ alkylene. In embodiments, L² is unsubstituted C₁-C₆ alkylene. In embodiments, L² is unsubstituted methylene. In embodiments, L² is unsubstituted C₂ alkylene. In embodiments, L² is unsubstituted C₃ alkylene. In embodiments, L² is unsubstituted C₄ alkylene. In embodiments, L² is unsubstituted C₅ alkylene. In embodiments, L² is unsubstituted C₆ alkylene. In embodiments, L² is R^{2A}-substituted C₁-C₆ or C₁-C₄ alkylene. In embodiments, L² is R^{2A}-substituted C₁-C₄ alkylene. In embodiments, L² is R^{2A}-substituted C₁-C₆ alkylene. In embodiments, L² is R^{2A}-substituted methylene. In embodiments, L² is R^{2A}-substituted C₂ alkylene. In embodiments, L² is R^{2A}-substituted C₃ alkylene. In embodiments, L² is R^{2A}-substituted C₄ alkylene. In embodiments, L² is R^{2A}-substituted C₅ alkylene. In embodiments, L² is R^{2A}-substituted C₆ alkylene. In embodiments, L² is R^{2A}-substituted 2 to 10 membered heteroalkylene. In embodiments, L² is R^{2A}-substituted 2 to 8 membered heteroalkylene. In embodiments, L² is R^{2A}-substituted 2 to 6 membered heteroalkylene. In embodiments, L² is R^{2A}-substituted 2 to 4 membered heteroalkylene. In embodiments, L² is an unsubstituted 2 to 10 membered heteroalkylene. In embodiments, L² is an unsubstituted 2 to 8 membered heteroalkylene. In embodiments, L² is an unsubstituted 2 to 6 membered heteroalkylene. In embodiments, L² is an unsubstituted 2 to 4 membered heteroalkylene. In embodiments, L² is unsubstituted C₂-C₆ alkenylene or C₂-C₄ alkenylene. In embodiments, L² is unsubstituted C₂ alkenylene. In embodiments, L² is unsubstituted C₃ alkenylene. In embodiments, L² is unsubstituted C₄ alkenylene. In embodiments, L² is unsubstituted C₅ alkenylene. In embodiments, L² is unsubstituted C₆ alkenylene. In embodiments, L² is R^{2A}-substituted C₂-C₆ or C₂-C₄ alkenylene. In embodiments, L² is R^{2A}-substituted C₂-C₄ alkenylene. In embodiments, L² is R^{2A}-substituted C₂-C₆ alkenylene. In embodiments, L² is R^{2A}-substituted C₂ alkenylene. In embodiments, L² is R^{2A}-substituted C₃ alkenylene. In embodiments, L² is R^{2A}-substituted C₄ alkenylene. In embodiments, L² is R^{2A}-substituted C₅ alkenylene. In embodiments, L² is R^{2A}-substituted C₆ alkenylene. In embodiments, L² is R^{2A}-substituted 2 to 10 membered heteroalkenylene. In embodiments, L² is R^{2A}-substituted 2 to 8 membered heteroalkenylene. In embodiments, L² is R^{2A}-substituted 2 to 6 membered heteroalkenylene. In embodiments, L² is R^{2A}-substituted 2 to 4 membered heteroalkenylene. In embodiments, L² is an unsubstituted 2 to 10 membered heteroalkenylene. In embodiments, L² is an unsubstituted 2 to 8 membered heteroalkenylene. In embodiments, L² is an unsubstituted 2 to 6 membered heteroalkenylene. In embodiments, L² is an unsubstituted 2 to 4 membered heteroalkenylene.

In embodiments, L² is R^{2A}-substituted or unsubstituted alkylene (e.g., C₁-C₂₀, C₁₀-C₂₀, C₁-C₅, C₁-C₆, or C₁-C₄), or R^{2A}-substituted or unsubstituted heteroalkylene (e.g., 2 to 20, 8 to 20, 2 to 10, 2 to 8, 2 to 6, or 2 to 4 membered). In embodiments, L² is substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20, 8 to 20, 2 to 10, 2 to 8, 2 to 6, or 2 to 4 membered), or substituted or unsubstituted alkenylene (e.g., C₂-C₆ alkenylene or C₂-C₄ alkenylene).

In an aspect is provided a composition and method including a prodrug having formula (I)

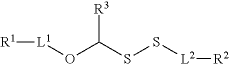

and a pharmaceutically acceptable excipient for diagnosis, monitoring, and treatment of a disease. In an aspect is provided a composition and method including a prodrug having formula (II)

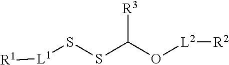

and a pharmaceutically acceptable excipient for diagnosis, monitoring, and treatment of a disease. In an aspect is provided a composition and method including a prodrug having formula (III)

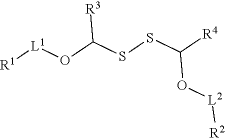

and a pharmaceutically acceptable excipient for diagnosis, monitoring, and treatment of a disease. For example, a composition of prodrug having formula (I), (II), or (III) may be formulated with pharmaceutically acceptable excipients including water, NaCl, normal saline solutions, lactated Ringer's, normal sucrose, normal glucose, binders, fillers, disintegrants, lubricants, coatings, sweeteners, flavors, hydb solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In an aspect is provided a pharmaceutical composition including a pharmaceutically acceptable excipient and a prodrug of formula (I)

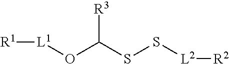

as described herein. In an aspect is provided a pharmaceutical composition including a pharmaceutically acceptable excipient and a prodrug of formula (II)

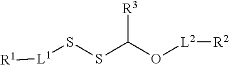

as described herein. In an aspect is provided a pharmaceutical composition including a pharmaceutically acceptable excipient and a prodrug of formula (III)

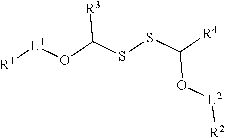

as described herein.

For use in the methods and/or applications (e.g., therapeutic applications) described herein, kits and articles of manufacture are also provided. In some embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from a variety of materials such as glass or plastic.

In an aspect is provided a kit. In embodiments, the kit includes a composition as described herein. In embodiments, the kit includes the reagents and containers useful for performing the methods as described herein. In embodiments, the kit includes the compound as described herein. In embodiments, the kit includes a plurality of the compounds described herein. In embodiments, the kit includes a compound described herein covalently bound to two drug moieties (e.g., wherein the first drug moiety is attached to L¹ and the second drug moiety is attached to L² of the compound described herein). In embodiments, the kit includes a compound described herein covalently bound to a drug moiety and a protein moiety (e.g., wherein the drug moiety is attached to L¹ and the protein moiety is attached to L² of the compound described herein). In embodiments, the kit includes a compound described herein covalently bound to a drug moiety and a nucleic acid moiety (e.g., wherein the drug moiety is attached to L¹ and the nucleic acid moiety is attached to L² of the compound described herein). In embodiments, the kit includes a compound described herein covalently bound to a drug moiety and a polymer moiety (e.g., wherein the drug moiety is attached to L¹ and the polymer moiety is attached to L² of the compound described herein). In embodiments, the kit includes a compound described herein covalently bound to a drug moiety and a nanoparticle (e.g., wherein the drug moiety is attached to L¹ and the nanoparticle is attached to L² of the compound described herein). In embodiments, the kit includes a compound described herein covalently bound to a drug moiety and a detectable agent (e.g., wherein the drug moiety is attached to L¹ and the detectable agent is attached to L² of the compound described herein).

### III. Methods

In an aspect is a method for diagnosing, monitoring, or treating a disease, including administering a therapeutically effective amount of a prodrug of formula (I), where the effective amount is sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). In embodiments, the disease is cancer. In embodiments, the cancer is lung cancer, colon cancer, colorectal cancer, pancreatic cancer, breast cancer, or leukemia. In embodiments, the cancer is lung cancer. In embodiments, the cancer is colon cancer. In embodiments, the cancer is colorectal cancer. In embodiments, the cancer is pancreatic cancer. In embodiments, the cancer is breast cancer. In embodiments, the cancer is leukemia. In embodiments, the disease is nervous system disease. In embodiments, the disease is infection. In embodiments, infection is bacterial infection. In embodiments, the disease is hypoxia. In embodiments, the disease is ischemia. In embodiments, the disease is inflammation. In embodiments, the disease is myocardial infarction. In embodiments, the disease is radiation damage. In embodiments, the disease is tissue damage. In embodiments, the disease is stroke.

In an aspect is a method for diagnosing, monitoring, or treating a disease, including administering a therapeutically effective amount of a prodrug of formula (II), where the effective amount is sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). In embodiments, the disease is cancer. In embodiments, the cancer is lung cancer, colon cancer, colorectal cancer, pancreatic cancer, breast cancer, or leukemia. In embodiments, the cancer is lung cancer. In embodiments, the cancer is colon cancer. In embodiments, the cancer is colorectal cancer. In embodiments, the cancer is pancreatic cancer. In embodiments, the cancer is breast cancer. In embodiments, the cancer is leukemia. In embodiments, the disease is nervous system disease. In embodiments, the disease is infection. In embodiments, infection is bacterial infection. In embodiments, the disease is hypoxia. In embodiments, the disease is ischemia. In embodiments, the disease is inflammation. In embodiments, the disease is myocardial infarction. In embodiments, the disease is radiation damage. In embodiments, the disease is tissue damage. In embodiments, the disease is stroke.

In an aspect is a method for diagnosing, monitoring, or treating a disease, including administering a therapeutically effective amount of a prodrug of formula (III), where the effective amount is sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). In embodiments, the disease is cancer. In embodiments, the cancer is lung cancer, colon cancer, colorectal cancer, pancreatic cancer, breast cancer, or leukemia. In embodiments, the cancer is lung cancer. In embodiments, the cancer is colon cancer. In embodiments, the cancer is colorectal cancer. In embodiments, the cancer is pancreatic cancer. In embodiments, the cancer is breast cancer. In embodiments, the cancer is leukemia. In embodiments, the disease is nervous system disease. In embodiments, the disease is hypoxia. In embodiments, the disease is ischemia. In embodiments, the disease is inflammation. In embodiments, the disease is myocardial infarction. In embodiments, the disease is radiation damage. In embodiments, the disease is tissue damage. In embodiments, the disease is stroke.

In embodiments, the method includes detecting the compound. In embodiments, detecting the compound (or absence thereof) indicates the presence (or absence) of the biomarker, and thus the status of the disease state of the subject.

In embodiments, the disease is an infectious disease, an autoimmune disease, hereditary disease, or cancer. In embodiments, the disease is an acute disease, a chronic disease (e.g., a malady that exists for greater than 6 months), an idiopathic disease, or a syndrome (e.g., Down syndrome). In embodiments, the disease is a relapsed disease (e.g., a malady that is detectable after a period of time of not being detectable).

In embodiments, the infectious disease is a disease or disorder associated with an infection from a pathogenic organism. In embodiments, the infectious disease is Acinetobacter infections, Actinomycosis, African sleeping sickness (African trypanosomiasis), AIDS (acquired immunodeficiency syndrome), Amoebiasis, Anaplasmosis, Angiostrongyliasis, Anisakiasis, Anthrax, Arcanobacterium haemolyticum infection, Argentine hemorrhagic fever, Ascariasis, Aspergillosis, Astrovirus infection, Babesiosis, Bacillus cereus infection, Bacterial meningitis, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and Infant botulism), Brazilian hemorrhagic fever, Brucellosis, Bubonic plague, Burkholderia infection, Buruli ulcer, Calicivirus infection (Norovirus and Sapovirus), Campylobacteriosis, Candidiasis (Moniliasis; Thrush), Capillariasis, Carrion's disease, Cat-scratch disease, Cellulitis, Chagas disease (American trypanosomiasis), Chancroid, Chickenpox, Chikungunya, Chlamydia, Chlamydophila pneumoniae infection (Taiwan acute respiratory agent or TWAR), Cholera, Chromoblastomycosis, Chytridiomycosis, Clonorchiasis, Clostridium difficile colitis, Coccidioidomycosis, Colorado tick fever (CTF), Common cold (Acute viral rhinopharyngitis; Acute coryza), Coronavirus disease 2019 (COVID-19), Creutzfeldt-Jakob disease (CJD), Crimean-Congo hemorrhagic fever (CCHF), Cryptococcosis, Cryptosporidiosis, Cutaneous larva migrans (CLM), Cyclosporiasis, Cysticercosis, Cytomegalovirus infection, Dengue fever, Desmodesmus infection, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum (Sixth disease), Fasciolasis, Fasciolopsiasis, Fatal familial insomnia (FFI), Filariasis, Food poisoning by Clostridium perfringens, Free-living amebic infection, Fusobacterium infection, Gas gangrene (Clostridial myonecrosis), Geotrichosis, Gerstmann-Straussler-Scheinker syndrome (GSS), Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome (HPS), Heartland virus disease, Helicobacter pylori infection, Hemolytic-uremic syndrome (HUS), Hemorrhagic fever with renal syndrome (HFRS), Hendra virus infection, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis (HGA), Human metapneumovirus infection, Human monocytic ehrlichiosis, Human papillomavirus (HPV) infection, Human parainfluenza virus infection, Hymenolepiasis, Epstein-Barr virus infectious mononucleosis (Mono), Influenza (flu), Isosporiasis, Kawasaki disease, Keratitis, Kingella kingae infection, Kuru, Lassa fever, Legionellosis (Legionnaires' disease), Pontiac fever, Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Middle East respiratory syndrome (MERS), Melioidosis (Whitmore's disease), Meningitis, Meningococcal disease, Metagonimiasis, Microsporidiosis, Molluscum contagiosum (MC), Monkeypox, Mumps, Murine typhus (Endemic typhus), Mycoplasma pneumonia, Mycoplasma genitalium infection, Mycetoma, Myiasis, Neonatal conjunctivitis (Ophthalmia neonatorum), Nipah virus infection, Norovirus, Variant Creutzfeldt-Jakob disease (vCJD, nvCJD), Nocardiosis, Onchocerciasis (River blindness), Opisthorchiasis, Paracoccidioidomycosis (South American blastomycosis), Paragonimiasis, Pasteurellosis, Pediculosis capitis (Head lice), Pediculosis corporis (Body lice), Pediculosis pubis (pubic lice, crab lice), Pelvic inflammatory disease (PID), Pertussis (whooping cough), Plague, Pneumococcal infection, Pneumocystis pneumonia (PCP), Pneumonia, Poliomyelitis, Prevotella infection, Primary amoebic meningoencephalitis (PAM), Progressive multifocal leukoencephalopathy, Psittacosis, Q fever, Rabies, Relapsing fever, Respiratory syncytial virus infection, Rhinosporidiosis, Rhinovirus infection, Rickettsial infection, Rickettsialpox, Rift Valley fever (RVF), Rocky Mountain spotted fever (RMSF), Rotavirus infection, Rubella, Salmonellosis, Severe acute respiratory syndrome (SARS), Scabies, Scarlet fever, Schistosomiasis, Sepsis, Shigellosis (bacillary dysentery), Shingles (Herpes zoster), Smallpox (variola), Sporotrichosis, Staphylococcal food poisoning, Staphylococcal infection, Strongyloidiasis, Subacute sclerosing panencephalitis, Bejel, Syphilis, and Yaws, Taeniasis, Tetanus (lockjaw), Tinea barbae (barber's itch), Tinea capitis (ringworm of the scalp), Tinea corporis (ringworm of the body), Tinea cruris (Jock itch), Tinea manum (ringworm of the hand), Tinea nigra, Tinea pedis (athlete's foot), Tinea unguium (onychomycosis), Tinea versicolor (Pityriasis versicolor), Toxic shock syndrome (TSS), Toxocariasis (ocular larva migrans (OLM)), Toxocariasis (visceral larva migrans (VLM)), Toxoplasmosis, Trachoma, Trichinosis, Trichomoniasis, Trichuriasis (whipworm infection), Tuberculosis, Tularemia, Typhoid fever, Typhus fever, Ureaplasma urealyticum infection, Valley fever, Venezuelan equine encephalitis, Venezuelan hemorrhagic fever, Vibrio vulnificus infection, Vibrio parahaemolyticus enteritis, Viral pneumonia, West Nile fever, White piedra (tinea blanca), Yersinia pseudotuberculosis infection, Yersiniosis, Yellow fever, Zeaspora, Zika fever, or Zygomycosis.

In embodiments, the disease is a nervous system disease. In embodiments, the nervous system disease is Amyotrophic Lateral Sclerosis. In embodiments, the nervous system disease is dopaminergic dysfunction. In embodiments, the nervous system disease is head injury. In embodiments, the nervous system disease is multiple sclerosis. In embodiments, the nervous system disease is muscular dystrophy. In embodiments, the nervous system disease is spinal cord dysfunction. In embodiments, the nervous system disease are diseases affecting subarachnoid space for autoimmune disease. In embodiments, the nervous system disease are diseases affecting the cerebellum.

In embodiments, the disease is inflammation. In embodiments, inflammation is chronic inflammation. In embodiments, inflammation is chronic wounds. In embodiments, inflammation is inflamed central nervous system. In embodiments, inflammation is subcutaneous wounds.

In embodiments, the disease is an autoimmune disease. In embodiments, the autoimmune disease is arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, or atopic dermatitis. In embodiments, the autoimmune disease is Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Bald disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, or Vogt-Koyanagi-Harada Disease.

In embodiments, the disease is a hereditary disease. In embodiments, the hereditary disease is cystic fibrosis, alpha-thalassemia, beta-thalassemia, sickle cell anemia (sickle cell disease), Marfan syndrome, fragile X syndrome, Huntington's disease, or hemochromatosis.

In an aspect is provided a method making a prodrug (e.g., a prodrug as described herein). In embodiments, the method includes synthesizing a prodrug having the formula (I):

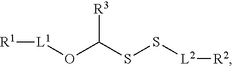

wherein R¹, R², and R³ are as described herein. In embodiments, the method includes synthesizing a prodrug having the formula (II):

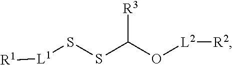

wherein R¹, R², and R³ are as described herein. In embodiments, the method includes synthesizing a prodrug having the formula (III):

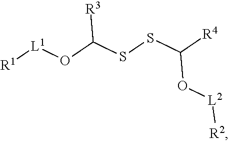

wherein R¹, R², R³, and R⁴ are as described herein. In embodiments, the method includes mixing a drug and a thiolation reagent together in a reaction vessel. In embodiments, the thiolation reagent is AcSH (thioacetic acid) or S-methyl methanesulfonothioate

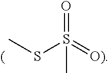

In embodiments, the method includes the synthetic protocol provided in the schemes provided herein (e.g., Schemes 3-7). In embodiments, the thiolation reagent is

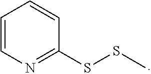

In embodiments, the thiolation reagent is

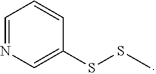

In embodiments, the thiolation reagent is

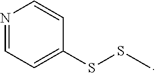

In embodiments, the thiolation reagent is

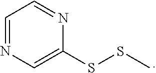

In embodiments, the thiolation reagent is

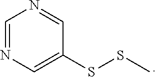

In embodiments, the thiolation reagent is

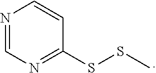

In embodiments, the thiolation reagent is

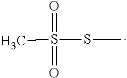

In embodiments, the thiolation reagent is

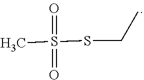

In embodiments, the thiolation reagent is a compound (e.g., a reagent) described in Mandal and Basu (RSC Adv., 2014, 4, 13854) or Musiejuk and Witt (Organic Preparations and Procedures International, 47:95-131, 2015), which are incorporated by reference in their entirety for all purposes. In embodiments, the thiolation reagent is K-thiotosylate. In embodiments, the thiolation reagent is NaSCH₃. In embodiments, the method of synthesizing a prodrug includes mixing the drug and TMSOTf, collidine, K-thiotosylate, 18-crown-6, and NaSMe; followed by purification (e.g., HPLC).

### EXAMPLES

### Example 1. Disulfide-Based Functional Groups for Biological Applications

Prodrugs refer to molecules with minimal measurable biological activity that may be metabolized into a biologically active molecule upon enzymatic, chemical, or a combination of both enzymatic and chemical conversion in vivo. As a result, prodrugs are enticing therapeutics due to the ability to selectively activate the compounds as needed. For example, the antibacterial agent Sultamicillin® includes an ampicillin moiety linked to a β-lactamase inhibitor with a diester bond, that is hydrolyzed in vivo to release the two compounds to treat bacterial infections such as β-lactamase producing bacteria (e.g., ampicillin-resistant H. influenzae) of the upper and lower respiratory tract. Redox responsive disulfide bonds are being exploited in this field, due to an abundance of glutathione, thioredoxin, cysteine, and thioredoxin reductase in cells and organisms. However, common approaches to attaching disulfide moieties to bioactive agents suffers from bulky steric hinderance and slow reaction kinetics.

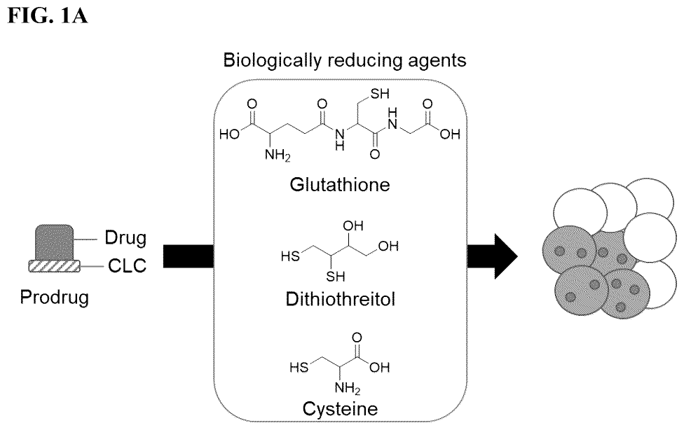

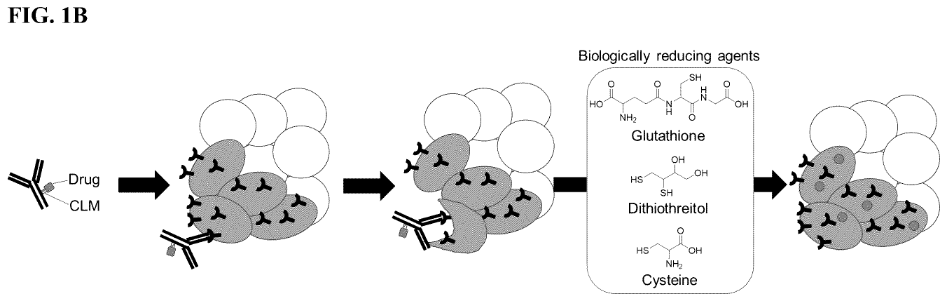

Disulfide bonds are important redox-reactive bonds and play an important role in protein stability (i.e., cysteine-cysteine disulfide bond formation) and/or signaling pathways. Glutathione, a cysteine containing tripeptide, is about 2 to 10 μM in blood and other body fluids, and approximately 1 to 10 μM within cellular cytosol (Lee et al. Chem. Rev. 2013, 113, 7, 5071-5109). Importantly, glutathione has been recognized as a biomarker of tumor development, associated with a 100-fold increase in glutathione concentration within tumor tissue and a 7-fold increase in cancer cells relative to normal tissues and cells (Russo et al. Cancer Res (1986) 46 (6): 2845-2848), enabling disulfide containing materials to be preferentially activated within diseased cells and tissues. Accordingly, therapeutic prodrugs containing chemically activatable groups (i.e., a cleavable linking cap, CLC) or therapeutics conjugated to a secondary agent (e.g., label, peptide, antibody) via a cleavable linker moiety (CLM), as illustrated in FIGS. 1A-1B, show promise in detecting and treating diseases.

During the years 2008-2018, a total of 287 new molecular entities were FDA approved, 33 of which were prodrugs. Some of these prodrugs include ceftaroline fosamil, dabigatran etexilate, fingolimod, abiraterone acetate, azilsartan medoximil, gabapentin enacarbil, tafluprost, sofosbuvir, dimethyl fumarate, eslicarbazepine acetate, droxidopa, tedizolid phosphate, isavuconazonium, sacubitril, uridine triacetate, aripiprazole lauroxil, tenofovir alafenamide, ixazomid, selexipag, deflazacort, telotristat etiprate, valbenazine, benznidazole, secnidazole, latanoprosteneo, fostamatinib, fosnetupitant, and baloxavir marboxil. Table 1 provides a collection of successful prodrugs and their corresponding tradenames.

TABLE 1
Commercially available prodrugs and associated tradename and
active form. The prodrug modification is enclosed by a dashed circle.

| Tradename | Prodrug | Active form |
|---|---|---|
| Accupril ™ | 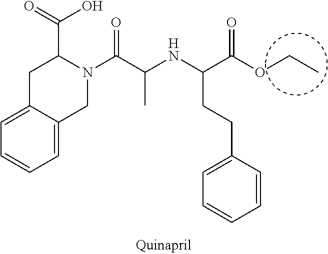 [SMILES: CCOC(=O)C(CCc1ccccc1)NC(C)C(=O)N1Cc2ccccc2CC1C(=O)O] | 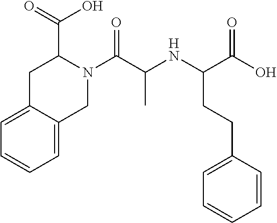 [SMILES: CC(NC(CCc1ccccc1)C(=O)O)C(=O)N1Cc2ccccc2CC1C(=O)O] |
| Acecol ™ | 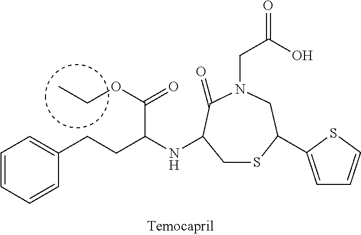 | 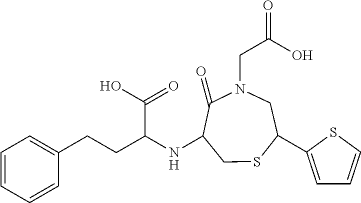 [SMILES: O=C(O)CN1CC(c2cccs2)SCC(NC(CCc2ccccc2)C(=O)O)C1=O] |
| Akynzeo ™ | 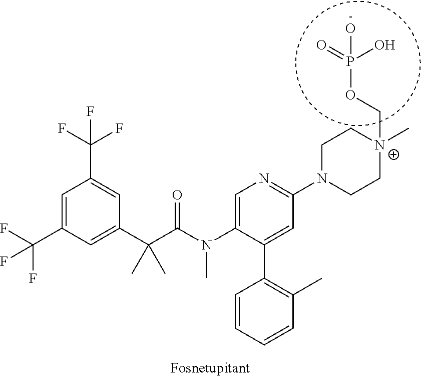 | 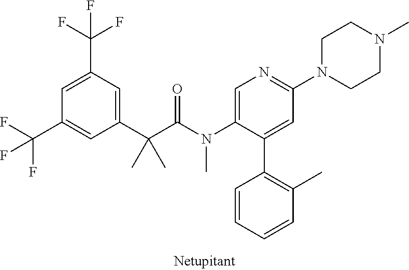 [SMILES: Cc1ccccc1-c1cc(N2CCN(C)CC2)ncc1N(C)C(=O)C(C)(C)c1cc(C(F)(F)F)cc(C(F)(F)F)c1] |
| Altace ™ | 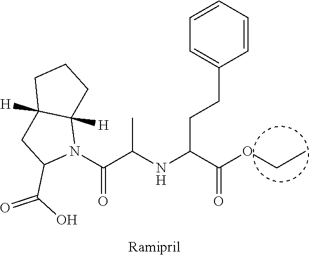 [SMILES: CCOC(=O)C(CCc1ccccc1)NC(C)C(=O)N1C(C(=O)O)C[C@@H]2CCC[C@@H]21] | 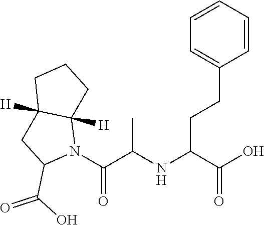 [SMILES: CC(NC(CCc1ccccc1)C(=O)O)C(=O)N1C(C(=O)O)C[C@@H]2CCC[C@@H]21] |
| Amias ™ | 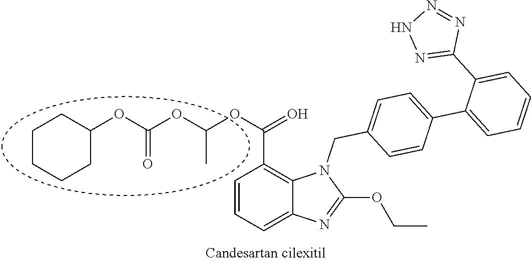 [SMILES: CCOc1nc2cccc(C(=O)OC(C)OC(=O)OC3CCCCC3)c2n1Cc1ccc(-c2ccccc2-c2nn[nH]n2)cc1] | 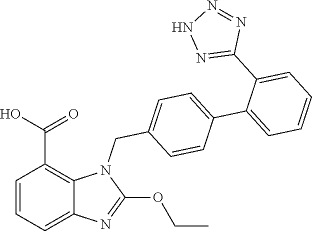 [SMILES: CCOc1nc2cccc(C(=O)O)c2n1Cc1ccc(-c2ccccc2-c2nn[nH]n2)cc1] |
| Aptiom ™ | 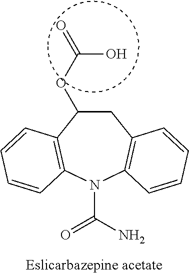 | 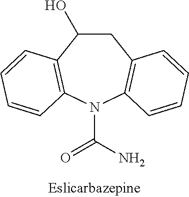 [SMILES: NC(=O)N1c2ccccc2CC(O)c2ccccc21] |
| Aristada ™ | 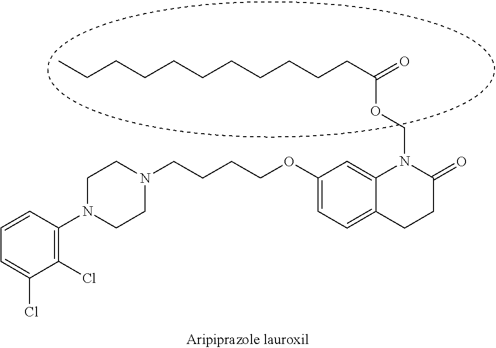 [SMILES: CCCCCCCCCCCC(=O)OCN1C(=O)CCc2ccc(OCCCCN3CCN(c4cccc(Cl)c4Cl)CC3)cc21] | 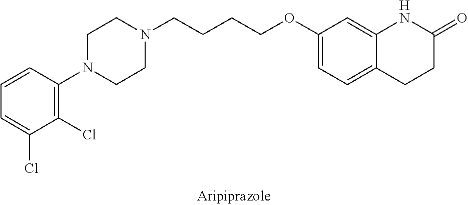 [SMILES: O=C1CCc2ccc(OCCCCN3CCN(c4cccc(Cl)c4Cl)CC3)cc2N1] |
| Candoxantril ™ | 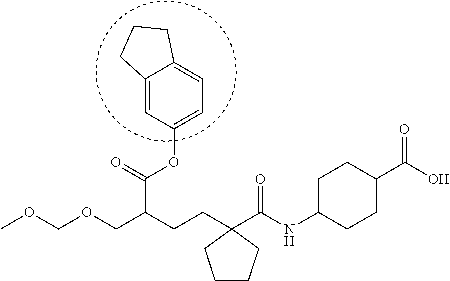 [SMILES: COCOCC(CCC1(C(=O)NC2CCC(C(=O)O)CC2)CCCC1)C(=O)Oc1ccc2c(c1)CCC2] | 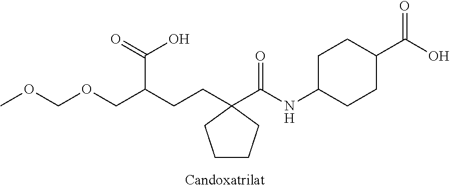 [SMILES: COCOCC(CCC1(C(=O)NC2CCC(C(=O)O)CC2)CCCC1)C(=O)O] |
| Coversyl Arginine | 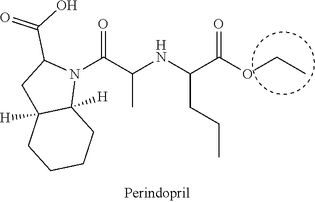 [SMILES: CCCC(NC(C)C(=O)N1C(C(=O)O)C[C@@H]2CCCC[C@@H]21)C(=O)OCC] | 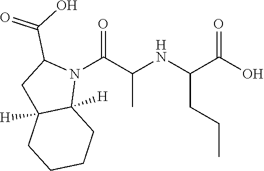 [SMILES: CCCC(NC(C)C(=O)N1C(C(=O)O)C[C@@H]2CCCC[C@@H]21)C(=O)O] |
| Cresemba ® | 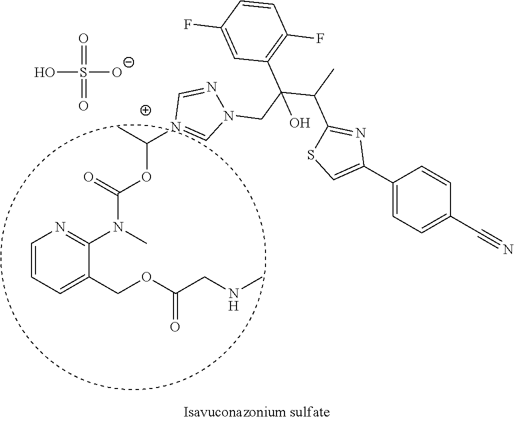 | 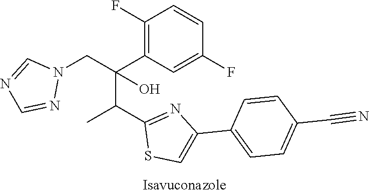 [SMILES: CC(c1nc(-c2ccc(C#N)cc2)cs1)C(O)(Cn1cncn1)c1cc(F)ccc1F] |
| Delapril | 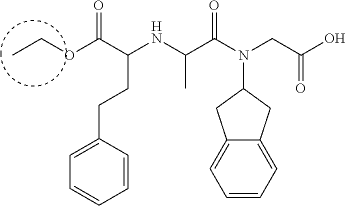 [SMILES: CCOC(=O)C(CCc1ccccc1)NC(C)C(=O)N(CC(=O)O)C1Cc2ccccc2C1] | 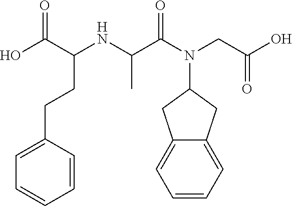 [SMILES: CC(NC(CCc1ccccc1)C(=O)O)C(=O)N(CC(=O)O)C1Cc2ccccc2C1] |
| Dynorm ™ | 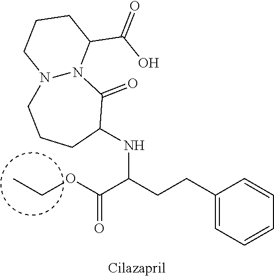 [SMILES: CCOC(=O)C(CCc1ccccc1)NC1CCCN2CCCC(C(=O)O)N2C1=O] | 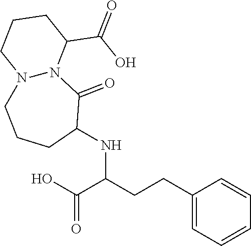 [SMILES: O=C(O)C(CCc1ccccc1)NC1CCCN2CCCC(C(=O)O)N2C1=O] |
| Edarbi ® | 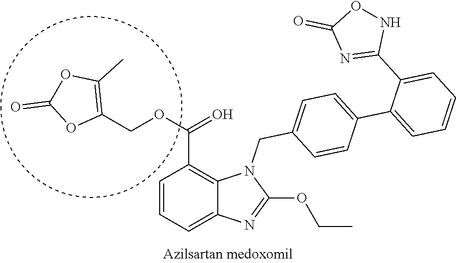 | 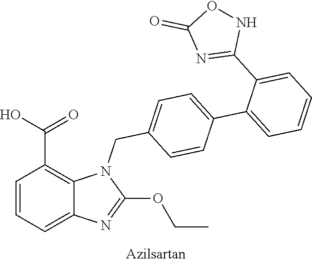 [SMILES: CCOc1nc2cccc(C(=O)O)c2n1Cc1ccc(-c2ccccc2-c2nc(=O)o[nH]2)cc1] |
| Effient ™ | 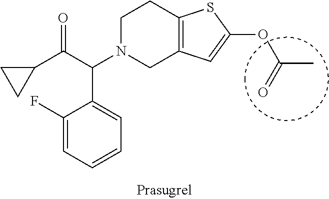 [SMILES: CC(=O)Oc1cc2c(s1)CCN(C(C(=O)C1CC1)c1ccccc1F)C2] | 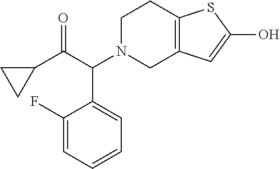 [SMILES: O=C(C1CC1)C(c1ccccc1F)N1CCc2sc(O)cc2C1] |
| Emend ™ | 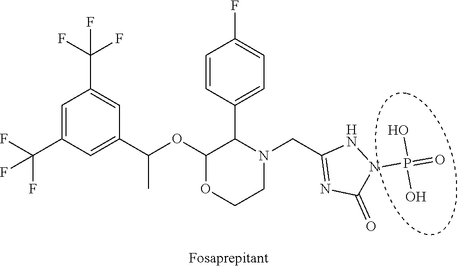 [SMILES: CC(OC1OCCN(Cc2nc(=O)n(P(=O)(O)O)[nH]2)C1c1ccc(F)cc1)c1cc(C(F)(F)F)cc(C(F)(F)F)c1] | 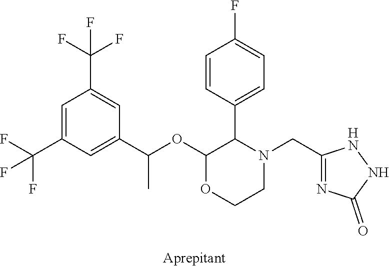 [SMILES: CC(OC1OCCN(Cc2nc(=O)[nH][nH]2)C1c1ccc(F)cc1)c1cc(C(F)(F)F)cc(C(F)(F)F)c1] |
| Emflaza ™ | 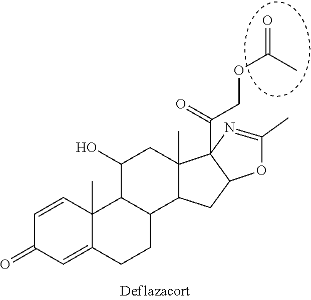 [SMILES: CC(=O)OCC(=O)C12N=C(C)OC1CC1C3CCC4=CC(=O)C=CC4(C)C3C(O)CC12C] | 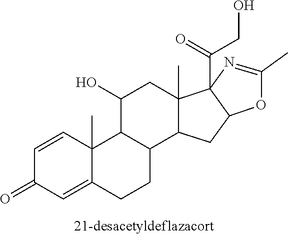 [SMILES: CC1=NC2(C(=O)CO)C(CC3C4CCC5=CC(=O)C=CC5(C)C4C(O)CC32C)O1] |
| Entresto ® | 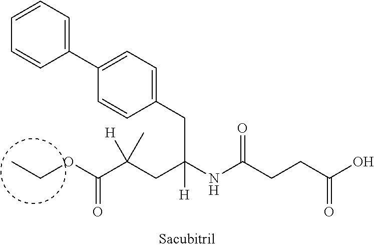 [SMILES: CCOC(=O)C(C)CC(Cc1ccc(-c2ccccc2)cc1)NC(=O)CCC(=O)O] | 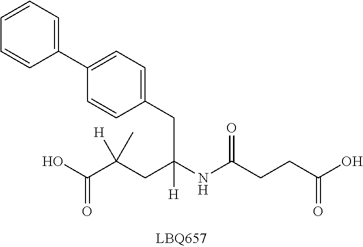 [SMILES: CC(CC(Cc1ccc(-c2ccccc2)cc1)NC(=O)CCC(=O)O)C(=O)O] |
| Epclusa ® | 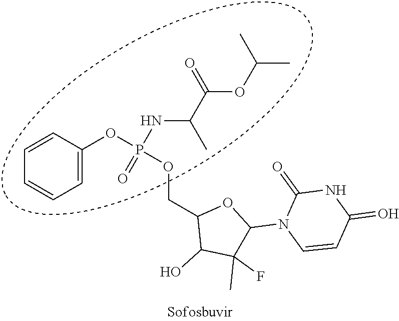 [SMILES: CC(C)OC(=O)C(C)NP(=O)(OCC1OC(n2ccc(=O)[nH]c2=O)C(C)(F)C1O)Oc1ccccc1] | 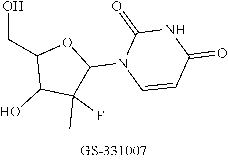 [SMILES: CC1(F)C(O)C(CO)OC1n1ccc(=O)[nH]c1=O] |
| Exanta ™ | 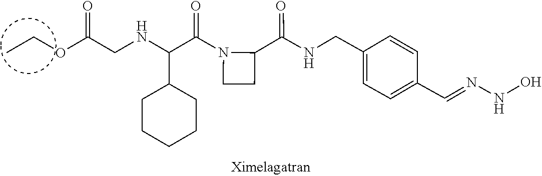 [SMILES: CCOC(=O)CNC(C(=O)N1CCC1C(=O)NCc1ccc(/C=N/NO)cc1)C1CCCCC1] | 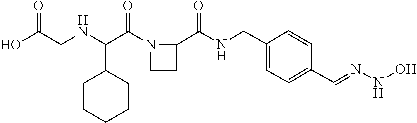 [SMILES: O=C(O)CNC(C(=O)N1CCC1C(=O)NCc1ccc(/C=N/NO)cc1)C1CCCCC1] |
| Famvir ™ | 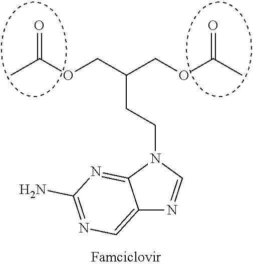 [SMILES: CC(=O)OCC(CCn1cnc2cnc(N)nc21)COC(C)=O] | 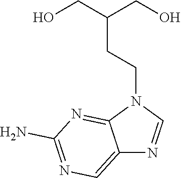 [SMILES: Nc1ncc2ncn(CCC(CO)CO)c2n1] |
| Genvoya ®/ Odefsey ®/ Descovy ® | 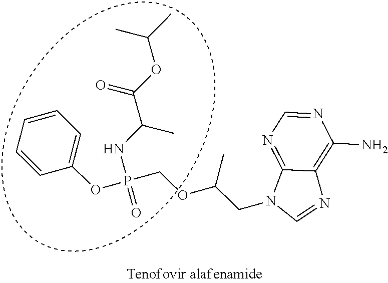 [SMILES: CC(C)OC(=O)C(C)NP(=O)(COC(C)Cn1cnc2c(N)ncnc21)Oc1ccccc1] | 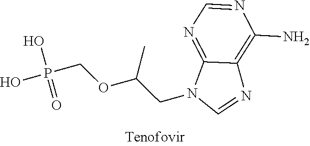 [SMILES: CC(Cn1cnc2c(N)ncnc21)OCP(=O)(O)O] |
| Geopen ® | 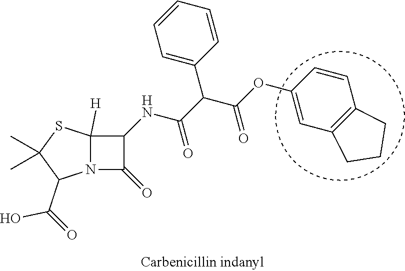 [SMILES: CC1(C)SC2C(NC(=O)C(C(=O)Oc3ccc4c(c3)CCC4)c3ccccc3)C(=O)N2C1C(=O)O] | 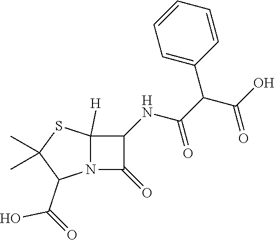 [SMILES: CC1(C)SC2C(NC(=O)C(C(=O)O)c3ccccc3)C(=O)N2C1C(=O)O] |
| Geocillin ® | 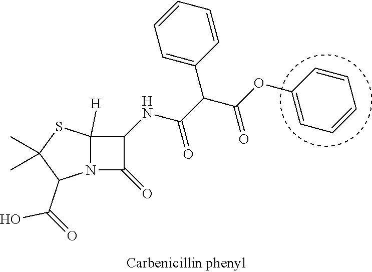 | 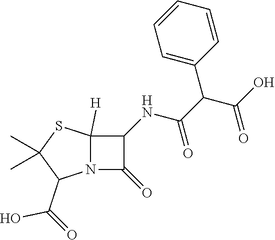 [SMILES: CC1(C)SC2C(NC(=O)C(C(=O)O)c3ccccc3)C(=O)N2C1C(=O)O] |
| Horizant ® | 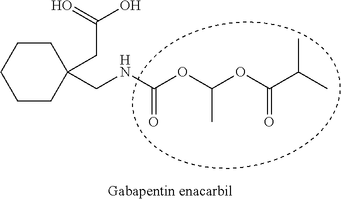 [SMILES: CC(OC(=O)NCC1(CC(=O)O)CCCCC1)OC(=O)C(C)C] | 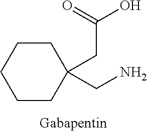 [SMILES: NCC1(CC(=O)O)CCCCC1] |
| Ingrezza ® | 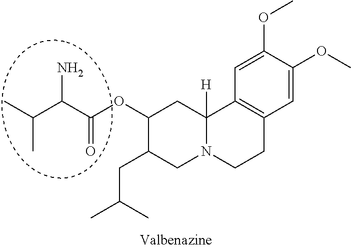 [SMILES: COc1cc2c(cc1OC)C1CC(OC(=O)C(N)C(C)C)C(CC(C)C)CN1CC2] | 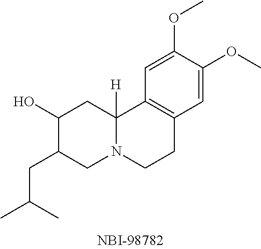 [SMILES: COc1cc2c(cc1OC)C1CC(O)C(CC(C)C)CN1CC2] |
| Invega Sustenna ® | 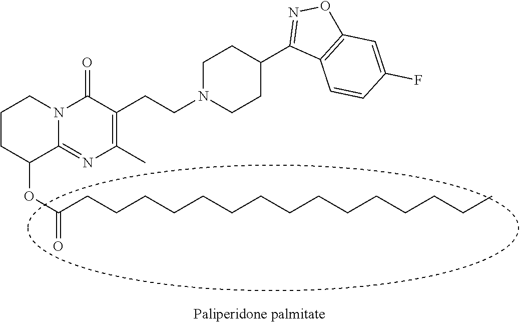 [SMILES: CCCCCCCCCCCCCCCC(=O)OC1CCCn2c1nc(C)c(CCN1CCC(c3noc4cc(F)ccc34)CC1)c2=O] | 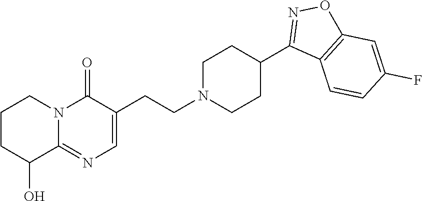 [SMILES: O=c1c(CCN2CCC(c3noc4cc(F)ccc34)CC2)cnc2n1CCCC2O] |
| Krintafel ® | 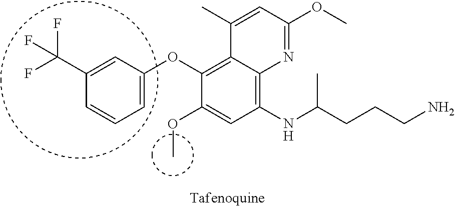 [SMILES: COc1cc(C)c2c(Oc3cccc(C(F)(F)F)c3)c(OC)cc(NC(C)CCCN)c2n1] | 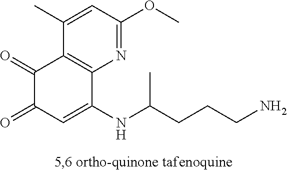 [SMILES: COc1cc(C)c2c(n1)C(NC(C)CCCN)=CC(=O)C2=O] |
| Lotensin ™ | 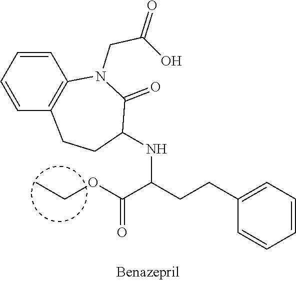 | 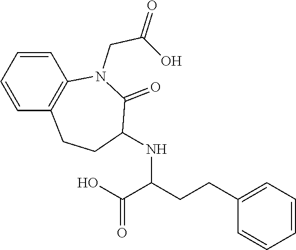 [SMILES: O=C(O)CN1C(=O)C(NC(CCc2ccccc2)C(=O)O)CCc2ccccc21] |
| Lusedra ® | 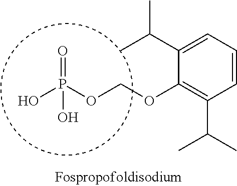 [SMILES: CC(C)c1cccc(C(C)C)c1OCOP(=O)(O)O] | 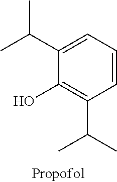 |
| Mavik ™ | 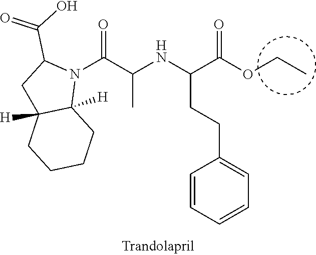 [SMILES: CCOC(=O)C(CCc1ccccc1)NC(C)C(=O)N1C(C(=O)O)C[C@H]2CCCC[C@@H]21] | 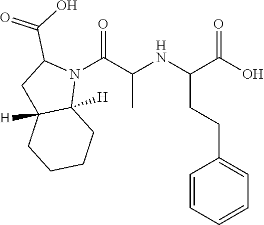 [SMILES: CC(NC(CCc1ccccc1)C(=O)O)C(=O)N1C(C(=O)O)C[C@H]2CCCC[C@@H]21] |
| Ninlaro ® | 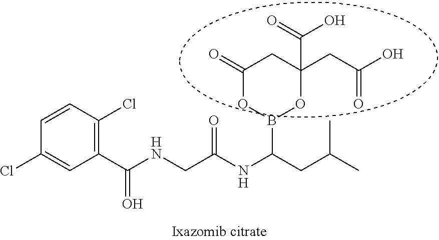 [SMILES: CC(C)CC(NC(=O)CNC(=O)c1cc(Cl)ccc1Cl)B1OC(=O)CC(CC(=O)O)(C(=O)O)O1] | 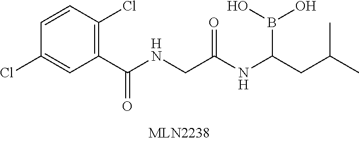 [SMILES: CC(C)CC(NC(=O)CNC(=O)c1cc(Cl)ccc1Cl)B(O)O] |
| Olmetec ™ | 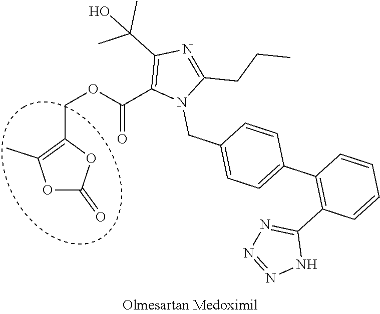 [SMILES: CCCc1nc(C(C)(C)O)c(C(=O)OCc2oc(=O)oc2C)n1Cc1ccc(-c2ccccc2-c2nnn[nH]2)cc1] | 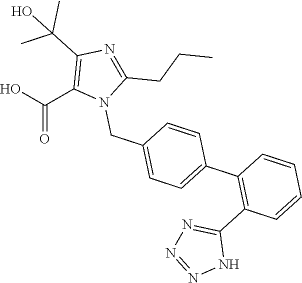 [SMILES: CCCc1nc(C(C)(C)O)c(C(=O)O)n1Cc1ccc(-c2ccccc2-c2nnn[nH]2)cc1] |
| Pondocillin ® | 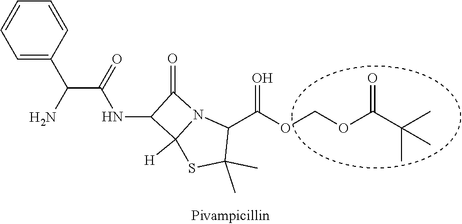 [SMILES: CC(C)(C)C(=O)OCOC(=O)C1N2C(=O)C(NC(=O)C(N)c3ccccc3)C2SC1(C)C] | 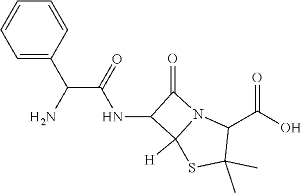 [SMILES: CC1(C)SC2C(NC(=O)C(N)c3ccccc3)C(=O)N2C1C(=O)O] |
| Pradaxa ® | 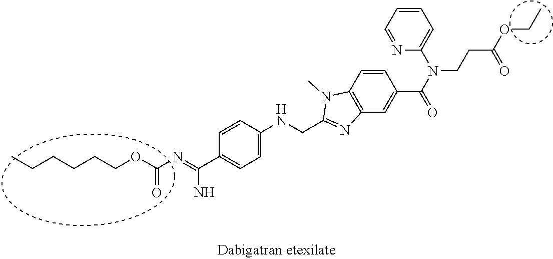 [SMILES: CCCCCCOC(=O)/N=C(\N)c1ccc(NCc2nc3cc(C(=O)N(CCC(=O)OCC)c4ccccn4)ccc3n2C)cc1] | 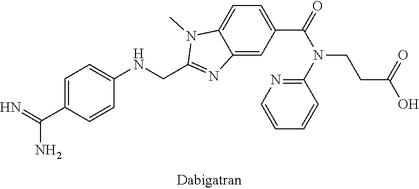 [SMILES: Cn1c(CNc2ccc(C(=N)N)cc2)nc2cc(C(=O)N(CCC(=O)O)c3ccccn3)ccc21] |
| Renormax | 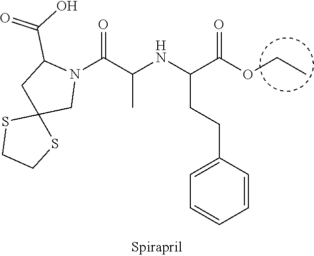 [SMILES: CCOC(=O)C(CCc1ccccc1)NC(C)C(=O)N1CC2(CC1C(=O)O)SCCS2] | 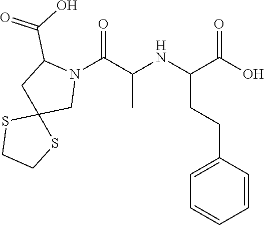 [SMILES: CC(NC(CCc1ccccc1)C(=O)O)C(=O)N1CC2(CC1C(=O)O)SCCS2] |
| Selexid ® | 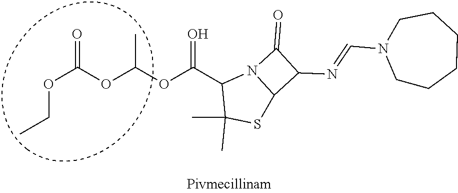 [SMILES: CCOC(=O)OC(C)OC(=O)C1N2C(=O)C(/N=C/N3CCCCCC3)C2SC1(C)C] | 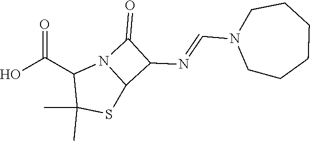 [SMILES: CC1(C)SC2C(/N=C/N3CCCCCC3)C(=O)N2C1C(=O)O] |
| Sivextro ® | 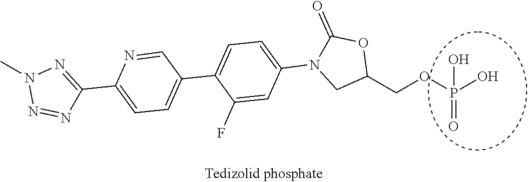 [SMILES: Cn1nnc(-c2ccc(-c3ccc(N4CC(COP(=O)(O)O)OC4=O)cc3F)cn2)n1] | 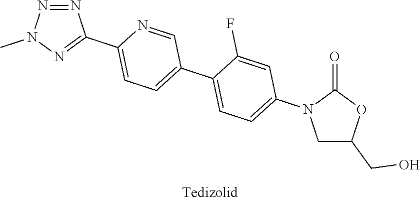 [SMILES: Cn1nnc(-c2ccc(-c3ccc(N4CC(CO)OC4=O)cc3F)cn2)n1] |
| Sovaldi ® | 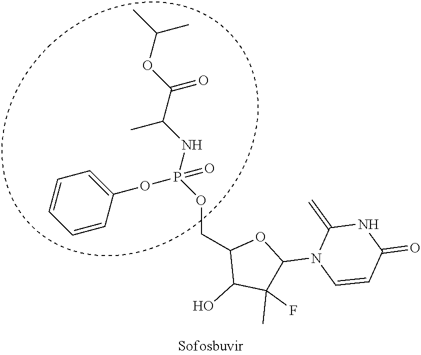 [SMILES: C=C1NC(=O)C=CN1C1OC(COP(=O)(NC(C)C(=O)OC(C)C)Oc2ccccc2)C(O)C1(C)F] | 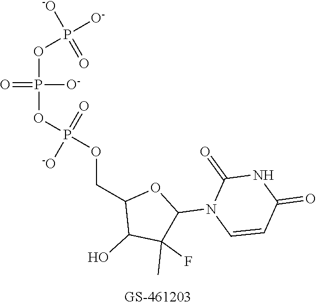 [SMILES: CC1(F)C(O)C(COP(=O)([O-])OP(=O)([O-])OP(=O)([O-])[O-])OC1n1ccc(=O)[nH]c1=O] |
| Spectracef ® | 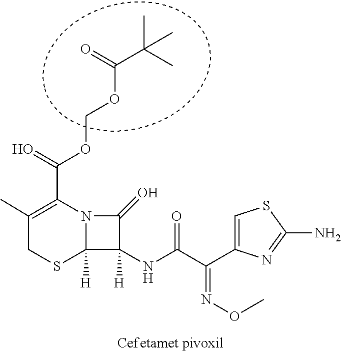 [SMILES: CO/N=C(/C(=O)N[C@@H]1C(=O)N2C(C(=O)OCOC(=O)C(C)(C)C)=C(C)CS[C@H]12)c1csc(N)n1] | 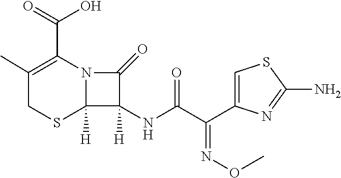 [SMILES: CO/N=C(/C(=O)N[C@@H]1C(=O)N2C(C(=O)O)=C(C)CS[C@H]12)c1csc(N)n1] |
| Spectrobid ® | 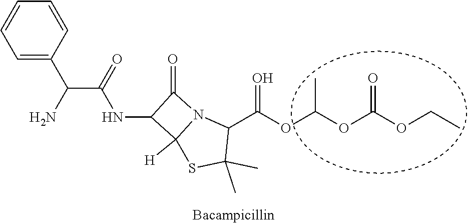 [SMILES: CCOC(=O)OC(C)OC(=O)C1N2C(=O)C(NC(=O)C(N)c3ccccc3)C2SC1(C)C] | 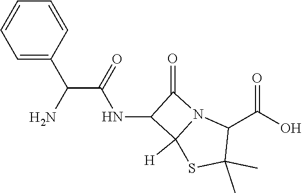 [SMILES: CC1(C)SC2C(NC(=O)C(N)c3ccccc3)C(=O)N2C1C(=O)O] |
| Tamiflu ® | 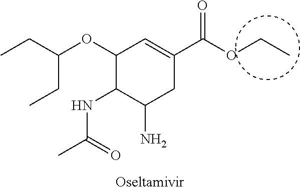 [SMILES: CCOC(=O)C1=CC(OC(CC)CC)C(NC(C)=O)C(N)C1] | 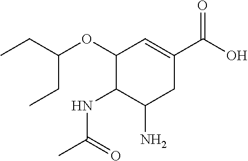 [SMILES: CCC(CC)OC1C=C(C(=O)O)CC(N)C1NC(C)=O] |
| Toviaz ™ | 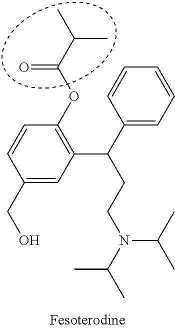 [SMILES: CC(C)C(=O)Oc1ccc(CO)cc1C(CCN(C(C)C)C(C)C)c1ccccc1] | 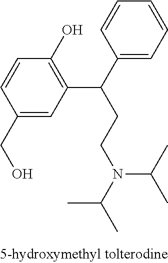 [SMILES: CC(C)N(CCC(c1ccccc1)c1cc(CO)ccc1O)C(C)C] |
| Univasc ™ | 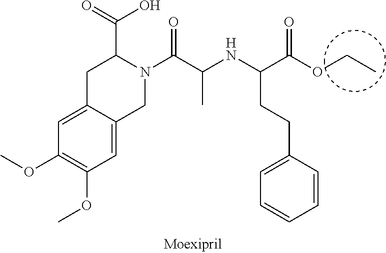 [SMILES: CCOC(=O)C(CCc1ccccc1)NC(C)C(=O)N1Cc2cc(OC)c(OC)cc2CC1C(=O)O] | 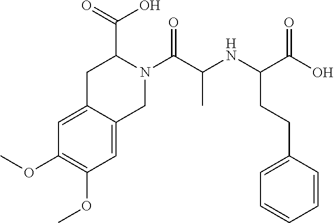 [SMILES: COc1cc2c(cc1OC)CN(C(=O)C(C)NC(CCc1ccccc1)C(=O)O)C(C(=O)O)C2] |
| Uptravi | 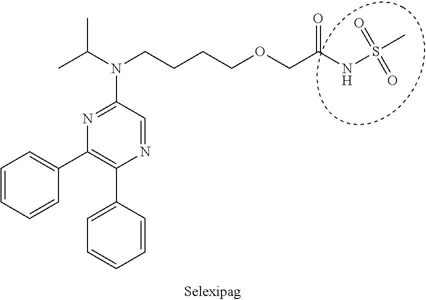 | 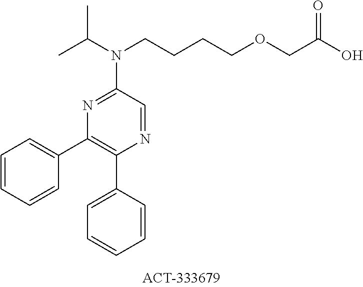 [SMILES: CC(C)N(CCCCOCC(=O)O)c1cnc(-c2ccccc2)c(-c2ccccc2)n1] |
| Valcyte ® | 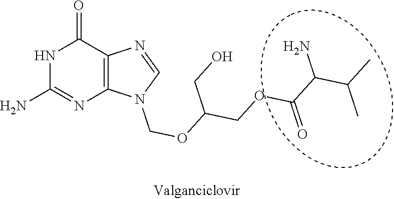 [SMILES: CC(C)C(N)C(=O)OCC(CO)OCn1cnc2c(=O)[nH]c(N)nc21] | 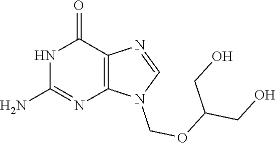 [SMILES: Nc1nc2c(ncn2COC(CO)CO)c(=O)[nH]1] |
| Valtrex ® | 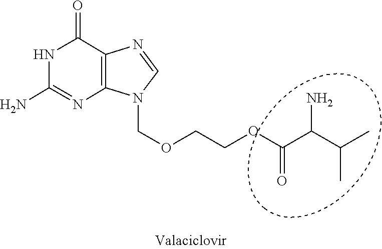 [SMILES: CC(C)C(N)C(=O)OCCOCn1cnc2c(=O)[nH]c(N)nc21] | 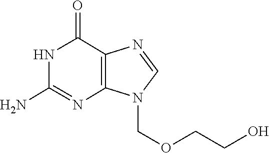 [SMILES: Nc1nc2c(ncn2COCCO)c(=O)[nH]1] |
| Vantin ® | 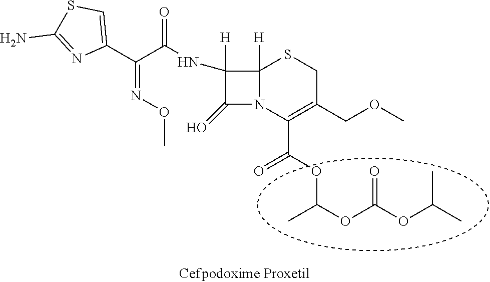 [SMILES: COCC1=C(C(=O)OC(C)OC(=O)OC(C)C)N2C(=O)C(NC(=O)/C(=N\OC)c3csc(N)n3)C2SC1] | 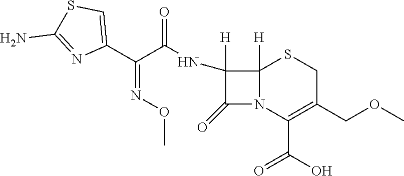 [SMILES: COCC1=C(C(=O)O)N2C(=O)C(NC(=O)/C(=N\OC)c3csc(N)n3)C2SC1] |
| Vasotec ™ | 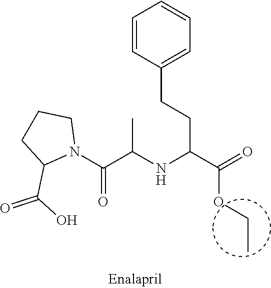 [SMILES: CCOC(=O)C(CCc1ccccc1)NC(C)C(=O)N1CCCC1C(=O)O] | 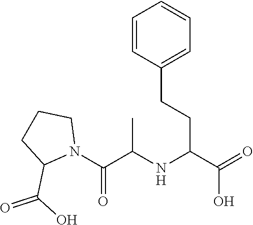 [SMILES: CC(NC(CCc1ccccc1)C(=O)O)C(=O)N1CCCC1C(=O)O] |
| Vyzulta ® | 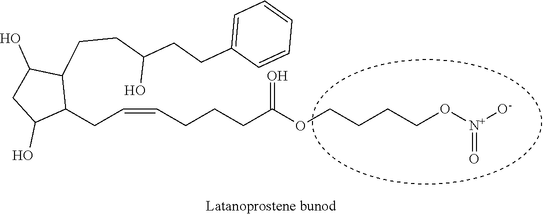 [SMILES: O=C(CCC/C=C\CC1C(O)CC(O)C1CCC(O)CCc1ccccc1)OCCCCO[N+](=O)[O-]] | 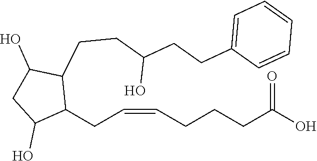 [SMILES: O=C(O)CCC/C=C\CC1C(O)CC(O)C1CCC(O)CCc1ccccc1] |
| Xermelo ® | 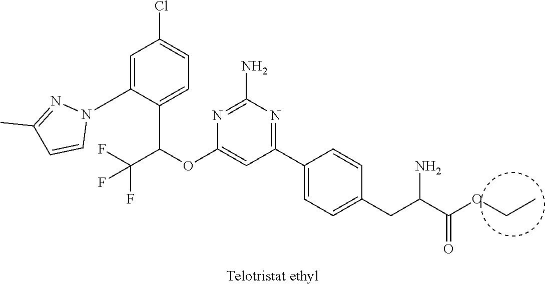 [SMILES: CCOC(=O)C(N)Cc1ccc(-c2cc(OC(c3ccc(Cl)cc3-n3ccc(C)n3)C(F)(F)F)nc(N)n2)cc1] | 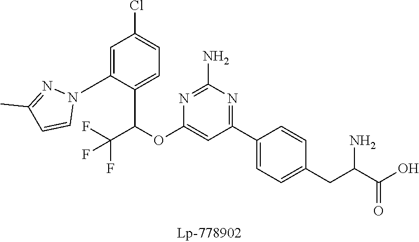 [SMILES: Cc1ccn(-c2cc(Cl)ccc2C(Oc2cc(-c3ccc(CC(N)C(=O)O)cc3)nc(N)n2)C(F)(F)F)n1] |
| Xofluza ® | 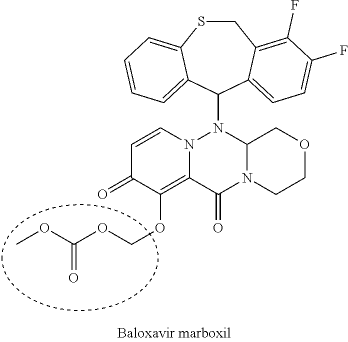 [SMILES: COC(=O)OCOc1c2n(ccc1=O)N(C1c3ccccc3SCc3c1ccc(F)c3F)C1COCCN1C2=O] | 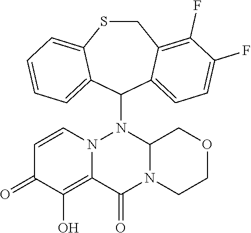 [SMILES: O=C1c2c(O)c(=O)ccn2N(C2c3ccccc3SCc3c2ccc(F)c3F)C2COCCN12] |
| Xuriden ® | 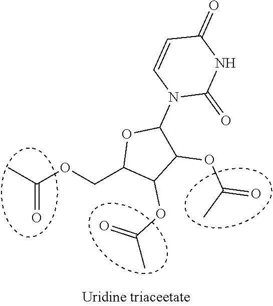 [SMILES: CC(=O)OCC1OC(n2ccc(=O)[nH]c2=O)C(OC(C)=O)C1OC(C)=O] | 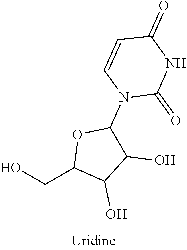 [SMILES: O=c1ccn(C2OC(CO)C(O)C2O)c(=O)[nH]1] |
| Zioptan ® | 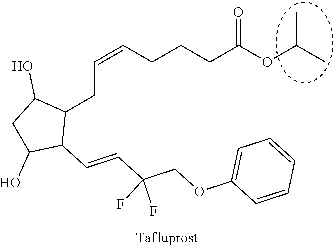 [SMILES: CC(C)OC(=O)CCC/C=C\CC1C(O)CC(O)C1/C=C/C(F)(F)COc1ccccc1] | 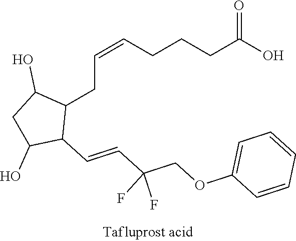 [SMILES: O=C(O)CCC/C=C\CC1C(O)CC(O)C1/C=C/C(F)(F)COc1ccccc1] |
| Zytiga ® | 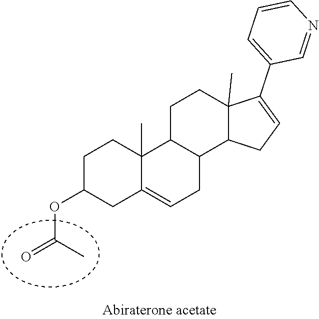 [SMILES: CC(=O)OC1CCC2(C)C(=CCC3C2CCC2(C)C(c4cccnc4)=CCC32)C1] | 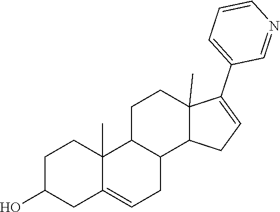 [SMILES: CC12CCC(O)CC1=CCC1C2CCC2(C)C(c3cccnc3)=CCC12] |

Previous attempts to derive therapeutics with CLM, such as dithiobenzyl carbamates (DTB), wherein upon introduction to the cell the disulfide is cleaved and the self-immolative cap degrades to release the bioavailable drug, as depicted in Scheme 1.

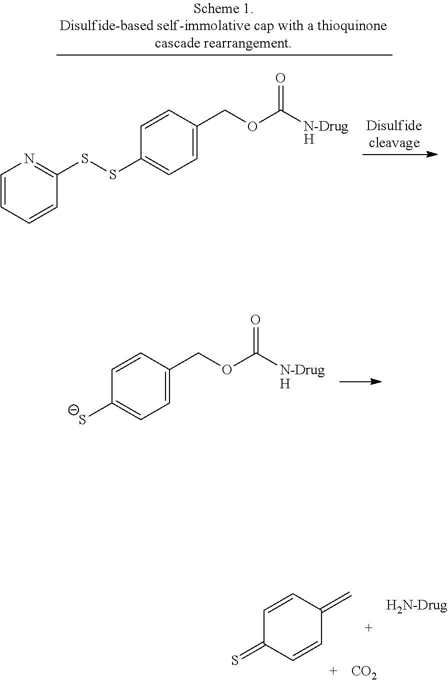

A person of ordinary skill in the art would understand the reaction mechanism in Scheme 1 also applies to therapeutics conjugated to a disulfide-based self-immolative moiety rather than self-immolative cap, where the self-immolative moiety links the therapeutic moiety to second therapeutic moiety, detectable agent, nanoparticle, polymer, or antibody or fragment thereof. Similar chemistry is used when deriving CLM therapeutic agents to attach small guiding ligands (e.g., toxins, antibodies, and cell-penetrating peptides) to facilitate targeted drug delivery. Exemplary examples of the aforementioned therapeutic agents include, but are not limited to, theranostics and antibody-drug conjugates (ADC).

Theranostics refer to an agent or a combination of two agents with diagnostic imaging and therapeutic capabilities. Radiotheranostics represent as an exemplary example of theranostics and typically harbor the following structural design: Cancer-targeting ligand-Linker-Radioisotope. The cancer-targeting ligand directs the radiotheranostic to the tumor site by binding to target receptors differentially expressed on the surface of tumor cells, which facilitates its internalization by the tumor cell. Following its entry, the radioisotope moiety induces radiative damage to the tumor cell. (Herrmann et al. Lancet Oncol. 2020 March; 21(3): e146-e156; Perera et al. J Nucl Med. 2022; 63(12):1793-1801). In 2022, the FDA approved Pluvicto™, a theranostic that contains a cancer-targeting ligand moiety capable of binding to prostate-specific membrane antigen (PMSA), radioactive payload ¹⁷⁷Lu, and a linker between the two moieties. Once localized within tumor cells, ¹⁷⁷Lu emits R particles, which induces cellular injury and DNA damage. The use of ¹⁷⁷Lu in this theranostic permits the evaluation of its localization and therapeutic efficacy using PET scans, and thus, enables ¹⁷⁷Lu to serve as a diagnostic imaging and therapeutic modality (Herrmann et al. Lancet Oncol. 2020 March; 21(3): e146-e156; Perera et al. J Nucl Med. 2022; 63(12):1793-1801). Additional examples of theranostics are known in the art, for example, Lee et al. Acc Chem Res. 2015 Nov. 17; 48(11): 2935-2946). This disclosure taught the use of theranostics developed from joining chemotherapeutic agents with fluorophores via a dithiodiethyl linker and their utility in evaluating tumor growth in vitro and in vivo.

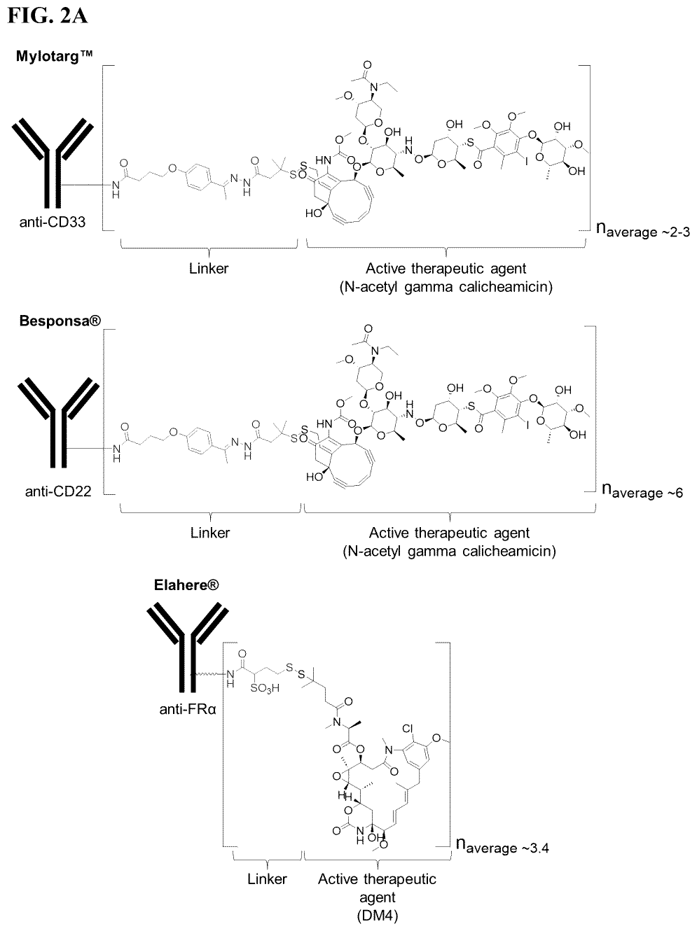

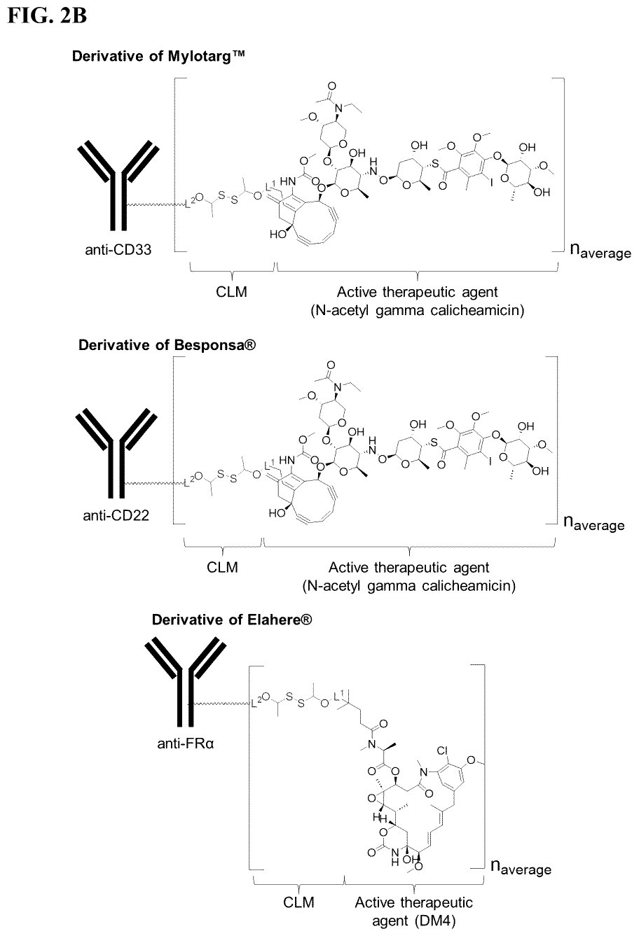

Antibody-drug conjugates are composed of a cytotoxic payload conjugated to tumor targeting monoclonal antibody (mAb) via chemical linker that is susceptible to self-immolation upon internalization by the tumor cell. As of 2023, the FDA approved thirteen antibody-drug conjugates, which include Mylotarg™, Adcentris®, Kadcyla®, Besponsa®, Lumoxiti®, Polivy®, Padcev®, Enhertu®, Trodelvy®, Blenrep, Zynlonta®, Tivdak®, and Elahere™. Gemtuzumab ozogamicin (Mylotarg™), inotuzumab ozogamicin (Besponsa®), and mirvetuximab soravtansine (Elahere™) are examples of disulfide linked antibody-drug conjugates; the structures of these disulfide containing ADCs are depicted in FIG. 2A. However, it has been reported that gemtuzumab ozogamicin (Mylotarg™) and inotuzumab ozogamicin (Besponsa®) suffer from the undesirable generation of byproducts and premature cleavage of the disulfide bonds (Srinivasarao M, Low P S. Chem Rev. 2017 Oct. 11; 117(19):12133-12164).

For disulfide-containing prodrugs to be selective and potent, the cleavable moiety should be (i) stable while in transit to the desired target (e.g., a cancer cell) and (ii) rapidly degradable to ensure efficient payload release. Chemistry originally developed for nucleic acid sequencing nucleotides may be applicable in developing redox responsive agents. In the context of nucleic acid sequencing, the use of nucleotides bearing a 3′ reversible terminator (RT) allows successive nucleotides to be incorporated into a polynucleotide chain in a controlled manner in a sequencing by synthesis approach. Sequencing by synthesis of nucleic acids ideally requires the controlled (i.e., one at a time), yet rapid, incorporation of the correct complementary nucleotide opposite the oligonucleotide being sequenced. This allows for accurate sequencing by adding nucleotides in multiple cycles as each nucleotide residue is sequenced one at a time, thus preventing an uncontrolled series of incorporations occurring. Nucleotides bearing a 3′ RT have been described in the literature, see for example U.S. Pat. Nos. 6,664,079, 10,738,072, 11,174,281, or Ju J. et al. (2006) Proc Natl Acad. Sci USA 103(52):19635-19640.

There are similar limitations for redox-responsive agents as for the reversible terminators. For example, the reversible terminator should prevent additional nucleotide molecules from being added to the polynucleotide while simultaneously being easily removable from the sugar moiety without causing damage to the polynucleotide or sequencing enzyme. Ideal reversible terminators therefore possess long term stability, can be efficiently incorporated by the sequencing enzyme, can prevent secondary or further nucleotide incorporation, and have the ability to be removed under mild conditions that do not cause damage to any sequencing component (e.g., nucleotides, primers, enzymes, polymers, etc.) preferably under aqueous conditions.

An important property of a redox-responsive agent is that it can be rapidly cleaved under appropriate conditions. Removal of a disulfide containing moiety or linker requires the formation of a thiol, followed by conversion to a hydroxide (see Scheme 2), via a tandem nucleophilic fragmentation reaction.

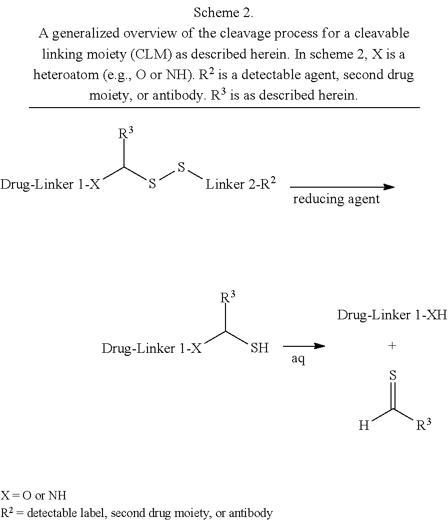

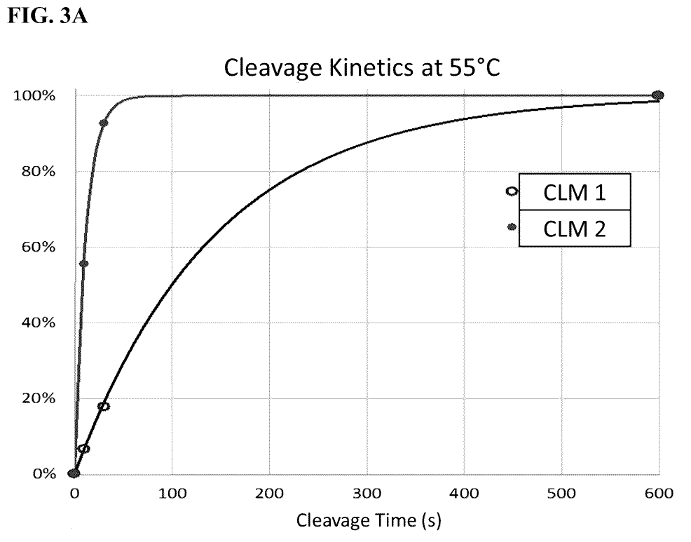

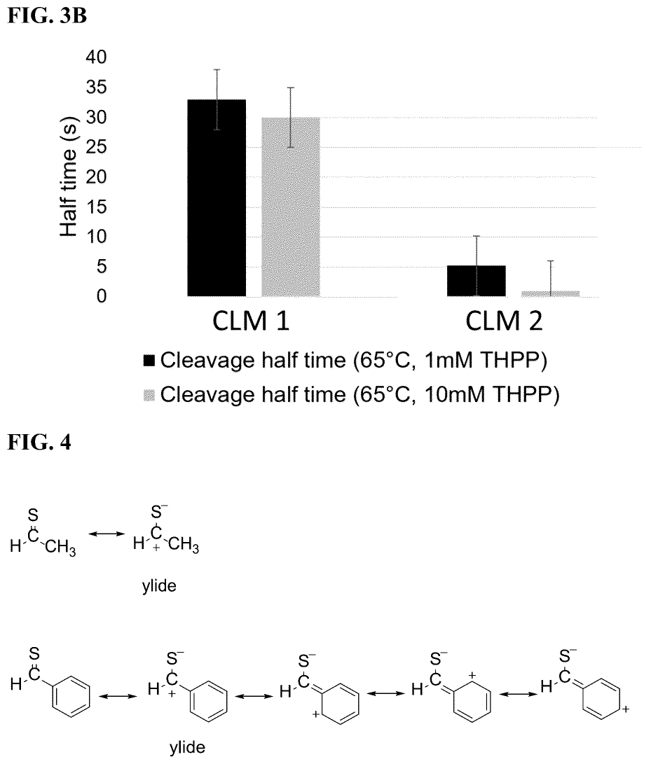

FIGS. 3A-3B shows the cleavage kinetics of two cleavable linking moieties, CLM #1 and CLM #2, for a model system. A person with ordinary skill in the art would understand that this model system could be extended to describe cleavage kinetics of compounds described herein by replacing the methyl group shown in both CLM #1 and CLM #2 with

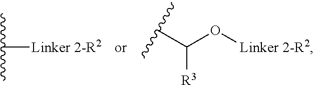

where R² and R³ are as described herein. CLM #1 is

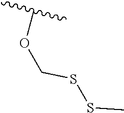 [SMILES: *OCSSC]

and CLM #2 has the formula

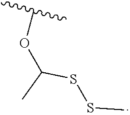 [SMILES: *OC(C)SSC]

For the assay, a plurality of compounds containing the CLM was immobilized on a solid support. Next, a cleavage solution containing 10 mM THPP as a reducing agent was introduced for controlled periods of time. A second labeled compound was introduced to the solid support; compounds without a CLM will bind the labeled compounds. The cleavage reaction was carried out at 55° C., in a buffer solution at 9.5 pH. The degree of cleavage was measured as the percent of compounds that include a labeled compound as a function of time. As seen in FIG. 3A, the cleavage occurs approximately 10-fold faster with CLM #2. This can be further observed by calculating the halftime, as depicted in FIG. 3B, which shows a drastic improvement in the kinetics (i.e., a reduction in halftime) of CLM #2 compared to CLM #1. While the cleavage of the disulfide bond (cleavable linking moiety) is rapid in both cases, the subsequent hydrolysis reaction that removes the residual portion is much faster with the new moiety (i.e., CLM #2). Modifying the reaction conditions (e.g., elevating the temperature to 65° C., modulating the pH, increasing the amount of reducing agent) results in faster cleavage. For example, data presented in FIG. 3B shows that by varying the amount of reducing agent and keeping the temperature constant at 65° C., the kinetics can be further improved. Increasing the amount of reducing agent THPP from 1 mM to 10 mM results in a further reduction in halftime of CLM #2 compared to CLM #1, within detection limits.

The data demonstrates the stability of the resultant thioaldehyde influences the cleavage rate of the thioacetal. For example, a CLM having the structure

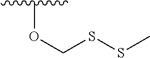 [SMILES: *OCSSC]

(referred to as a methylene disulfide, or CLM #1), wherein the oxygen is attached to drug agent, results in the formation of thioformaldehyde, a notoriously unstable molecule which rapidly oligomerizes to 1,3,5-trithiane. Thioformaldehydes are highly reactive and inherently unstable species due to the lack of steric and resonance stabilization afforded to the sp² carbon by the hydrogens. In accordance with the Hammond Postulate, the transition state is geometrically more similar to the thioaldehyde for this particular reaction (see for example March's Advanced Organic Chemistry, 6th Ed., Wiley, 2007, Michael B. Smith and Jerry March, Chapter 6 Methods of determining mechanisms, page 308). Conceptualizing the thioaldehyde with all available resonance geometries (see FIG. 4) suggests a stable ylide structure that is geometrically similar to the resultant thioaldehyde will be more thermodynamically favored. Therefore, increasing the resonance stabilization to the sp² carbon by including a resonance-stabilizing moiety (e.g., a cyclic moiety, such as an aromatic or heteroaromatic moiety) involves only a small reorganization of the molecular structures and thus permits faster cleavage. This concept is further illustrated in FIG. 4 and supported vide infra. For example, a CLM that includes a methyl substituent on the methylene carbon having the structure

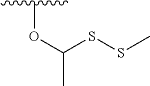 [SMILES: *OC(C)SSC]

(CLM #2) increases the cleavage rate approximately 10-fold relative to CLM #1. Additionally, merely extending the alkyl chain

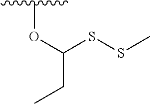 [SMILES: *OC(CC)SSC]

(CLM #2a) did not significantly improve the cleavage rate relative to CLM #2. Without wishing to be bound by theory, aliphatic thioaldehydes (e.g., such as the thioaldehyde produced when cleaving CLM #2 or CLM #2a) may polymerize and their isolation may be problematic, suggesting substituents which further stabilize the resultant thioaldehyde (i.e., have a greater number of resonant structures) results in an increased cleavage rate. Aromatic thioaldehydes are more stable (see Moldoveanu, S. Chapter 10—Pyrolysis of Aldehydes and Ketones, Pyrolysis of Organic Molecules (Second Edition), Elsevier, 2019, Pages 391-418), therefore the cleavage rate of disulfide-containing redox-responsive agents that produce aromatic thioaldehydes increases relative to a methylene disulfide. Thus, the modified compounds as described herein are stable and rapidly cleaved under mild conditions.

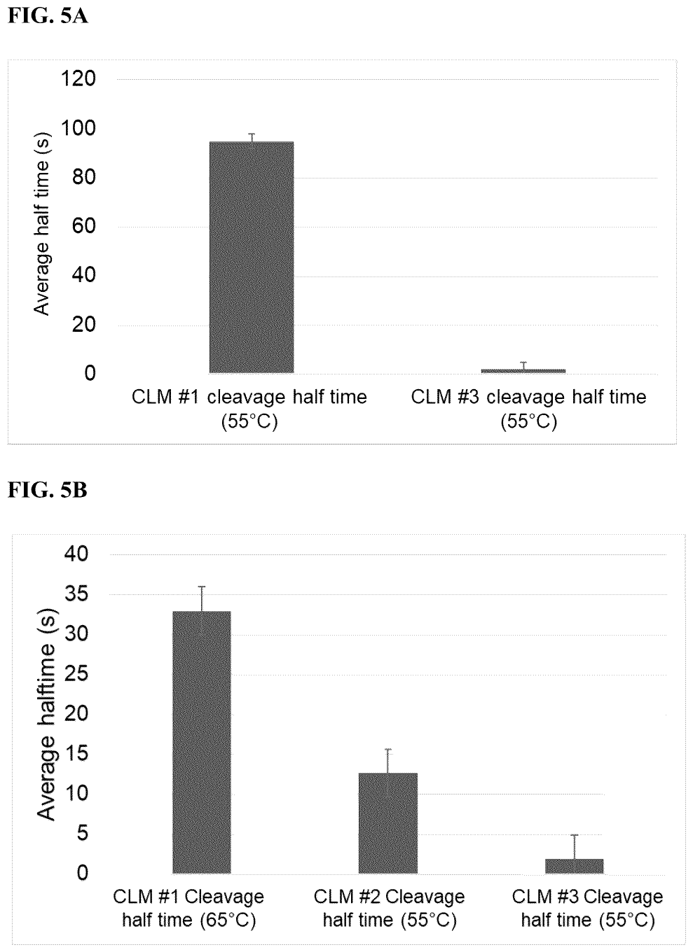

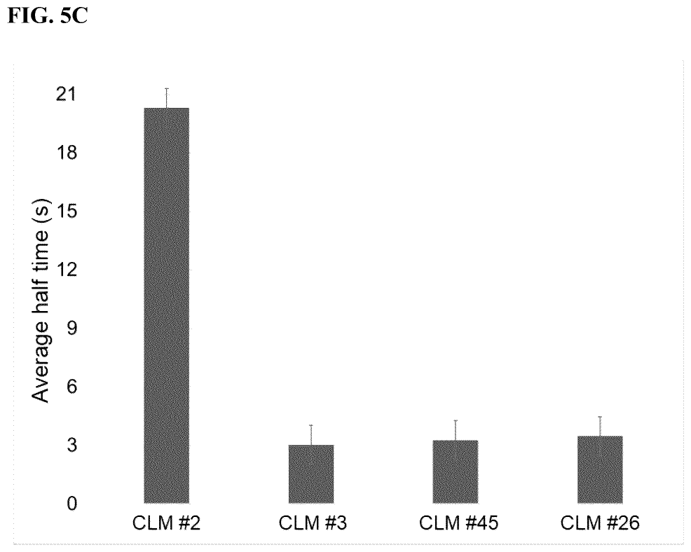

FIGS. 5A-5B reports the cleavage halftime rates for three different compounds containing cleavable linking moieties (CLMs). CLM #1 has the structure

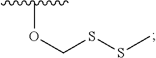 [SMILES: *OCSSC]

CLM #2 is the methyl-substituted methylene, e.g., having the formula

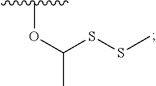 [SMILES: *OC(C)SSC]

### and CLM #3 is

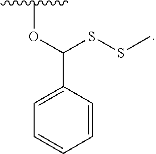 [SMILES: *OC(SSC)c1ccccc1]

A similar assay as described supra was performed, except that the concentration of the reducing agent was diluted to 1 mM of THPP and the cleavage reaction was carried out at 55° C. The cleavage reaction for CLM #1 was slow at 55° C., as shown in FIG. 5A, so to make a meaningful comparison to the other RT moieties, the temperature for only CLM #1 was increased to 65° C., as shown in FIG. 5B. As observed in FIGS. 5A-5C, there is a drastic improvement in the cleavage kinetics (i.e., a reduction in halftime) of CLM #3 compared to CLM #1 and CLM #2. While the cleavage of the disulfide bond (cleavable linking moiety) is rapid in both cases, the subsequent hydrolysis reaction that removes the residual portion is much faster with the CLM moieties described herein. Modifying the reaction conditions (e.g., elevating the temperature to 65° C., increasing the pH, increasing the amount or concentration of the reducing agent) results in faster cleavage.

The kinetics of the disulfide cleavage are not significantly affected by the terminal alkyl group. Data presented within Table 3 show that when R² is methyl and R¹ is methyl or ethyl, the kinetics are relatively invariant. Similarly, when R² is unsubstituted phenyl and R¹ is unsubstituted methyl, unsubstituted ethyl, or unsubstituted propyl, the cleavage kinetics are comparable. Advantageously, the cleavage kinetics for the compounds described herein are surprisingly 10× faster than control compounds (e.g., when R¹ and R² are methyl).

TABLE 3
Cleavage kinetics measurements varying R¹ and R² for dTTP
nucleotides. Cleavage conditions were measured using
0.1 mM THPP at 55° C.

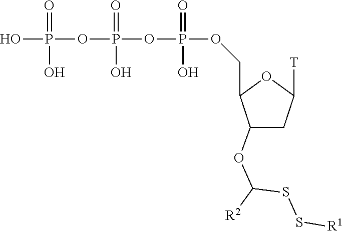

| | | Cleavage |
|---|---|---|
| R² | R¹ | halftime (s) |
| 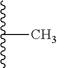 | 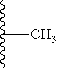 | 21.2 ± 3 |
| 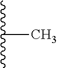 | 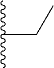 [SMILES: *CC] | 20.3 ± 3 |
| 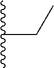 [SMILES: *CC] | 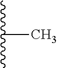 | 21.1 ± 3 |
| 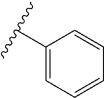 [SMILES: *c1ccccc1] | 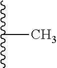 | 1.6 ± 3 |
| 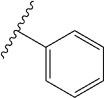 [SMILES: *c1ccccc1] | 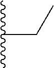 [SMILES: *CC] | 2.8 ± 3 |
| 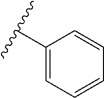 [SMILES: *c1ccccc1] | 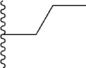 [SMILES: *CCC] | 2.9 ± 3 |
| 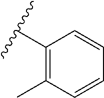 [SMILES: *c1ccccc1C] | 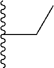 [SMILES: *CC] | 0.9 ± 3 |
| 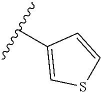 [SMILES: *c1ccsc1] | 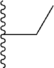 [SMILES: *CC] | 2.7 ± 3 |
| 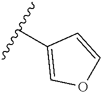 [SMILES: *c1ccoc1] | 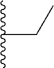 [SMILES: *CC] | 2.0 ± 3 |
| 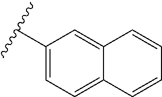 [SMILES: *c1ccc2ccccc2c1] | 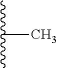 | 3.5 ± 3 |

As shown in Table 3, superior cleavage kinetics in the model system with dTTP nucleotides was observed when R² is an aromatic or heteroaromatic moiety. A person with ordinary skill in the art would understand that the data from Table 3 could be extended to describe the cleavage kinetics of the compounds (e.g., prodrugs) described herein. For example, when R³ and/or R⁴ of the compound described herein is an aromatic or heteroaromatic moiety, fast cleavage kinetics would be observed for the compounds described herein.

### Example 2. Thioaldehyde Stability

The kinetics of a reaction depend on the activation energy, i.e., the difference between the energy of the reactants and the transition state. However, transition states have only a transitory existence and are difficult, if not impossible, to observe, isolate, and quantify. A generalization to predicting reaction rates is provided in the Hammond Postulate, which suggests the activation energy of the rate determining step is inversely proportional to the stability of the transition state. In an endothermic reaction the transition state structure is closer to the structure of the products, and so it follows that a more stable product reflects a more stable transition state and has a lower activation energy.

Invoking the Hammond Postulate for the reaction of interest, i.e., the thiol bearing compound converting to the free OH (i.e., the activated prodrug) and a thioaldehyde, depicted in Scheme 3, posits the thermodynamic stability of the resultant thioaldehyde influences the cleavage rate. Simple thermodynamics provides the enthalpy changes of the reaction, ΔH, as a measure of the thermodynamic stability. The enthalpy change is calculated as the difference in the enthalpy of the products and reactants, ΔH=ΔH_{products}−ΔH_{reactants}.

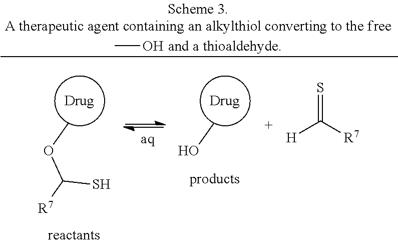

Using ΔH as a corollary for the reaction rate, it is possible to predict which cleavable linker moieties will cleave rapidly under suitable conditions. Gas phase calculations were performed using hybrid Density Functional Theory (B3LYP) with a large basis set (Valence triple-zeta with two sets of polarization functions) to determine the optimized structure and energy of the reactants and the products, which was used to derive a ΔH for a variety of compounds, see Table 4. Experimental evidence supports using ΔH as a proxy for the reaction rate, as reported in Example 1 and depicted in FIGS. 2 and 4 and Table 4, showing the experimentally derived cleavage halftime for CLM #1, CLM #2, and CLM #3 as a function of the calculated ΔH. Reducing the energetic burden on the system, i.e., reducing the enthalpy, corresponds to faster cleavage rates. For example, using the data presented in Table 4, the compound (e.g., prodrug) with CLM #1, which has a calculated ΔH value of 18.0 kcal/mol, is expected to exhibit a slower cleavage reaction rate compared to the compound with CLM #28, which has a calculated ΔH value of −1.2 kcal/mol. This is readily observed in FIGS. 2 and 4 showing that as the enthalpy decreases for CLM #1, CLM #2, and CLM #3, the cleavage halftime similarly reduces. In embodiments, the compound has an enthalpy of about 5 to about 12 kcal/mol.

TABLE 4
Calculated enthalpies for Scheme 3 reactions.

| | Reference | ΔH |
|---|---|---|
| R⁷ (of Scheme 3) | No. | (kcal/mol) |
| 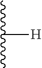 [SMILES: *[H]] | CLM #1 | 18.0 |
| 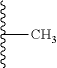 | CLM #2 | 13.5 |
| 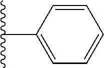 [SMILES: *c1ccccc1] | CLM #3 | 5.6 |
| 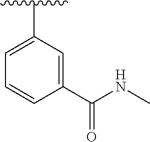 [SMILES: *c1cccc(C(=O)NC)c1] | CLM #4 | 4.1 |
| 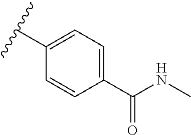 [SMILES: *c1ccc(C(=O)NC)cc1] | CLM #5 | 3.6 |
| 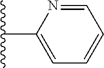 [SMILES: *c1ccccn1] | CLM #6 | 5.4 |
| 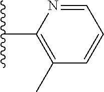 [SMILES: *c1ncccc1C] | CLM #7 | 6.6 |
| 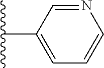 [SMILES: *c1cccnc1] | CLM #8 | 5.8 |
| 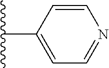 [SMILES: *c1ccncc1] | CLM #9 | 7.9 |
| 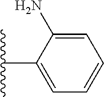 [SMILES: *c1ccccc1N] | CLM #10 | 6.8 |
| 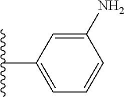 [SMILES: *c1cccc(N)c1] | CLM #11 | 5.7 |
| 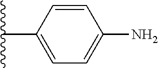 [SMILES: *c1ccc(N)cc1] | CLM #12 | 2.5 |
| 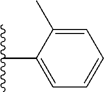 [SMILES: *c1ccccc1C] | CLM #13 | 6.3 |
| 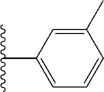 [SMILES: *c1cccc(C)c1] | CLM #14 | 5.5 |
| 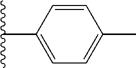 [SMILES: *c1ccc(C)cc1] | CLM #15 | 4.9 |
| 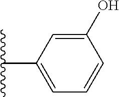 [SMILES: *c1cccc(O)c1] | CLM #16 | 6.3 |
| 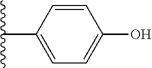 [SMILES: *c1ccc(O)cc1] | CLM #17 | 3.8 |
| 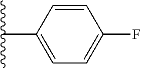 [SMILES: *c1ccc(F)cc1] | CLM #18 | 5.3 |
| 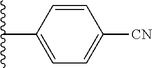 [SMILES: *c1ccc(C#N)cc1] | CLM #19 | 7.2 |
| 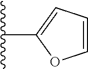 [SMILES: *c1ccco1] | CLM #20 | 3.7 |
| 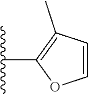 [SMILES: *c1occc1C] | CLM #21 | 2.2 |
| 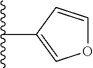 [SMILES: *c1ccoc1] | CLM #22 | 7.2 |
| 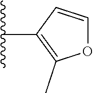 [SMILES: *c1ccoc1C] | CLM #23 | 3.0 |
| 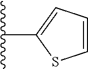 [SMILES: *c1cccs1] | CLM #24 | 2.7 |
| 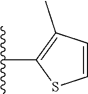 [SMILES: *c1sccc1C] | CLM #25 | 0.7 |
| 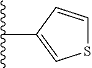 [SMILES: *c1ccsc1] | CLM #26 | 6.7 |
| 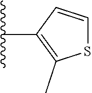 [SMILES: *c1ccsc1C] | CLM #27 | 6.3 |
| 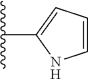 [SMILES: *c1ccc[nH]1] | CLM #28 | −1.2 |
| 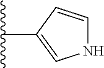 [SMILES: *c1cc[nH]c1] | CLM #29 | 4.2 |
| 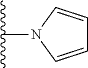 [SMILES: *n1cccc1] | CLM #30 | 0.1 |
| 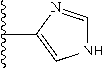 [SMILES: *c1c[nH]cn1] | CLM #31 | 4.9 |
| 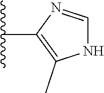 [SMILES: *c1nc[nH]c1C] | CLM #32 | 2.4 |
| 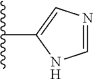 [SMILES: *c1cnc[nH]1] | CLM #33 | 0.8 |
| 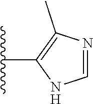 [SMILES: *c1[nH]cnc1C] | CLM #34 | −1.0 |
| 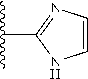 [SMILES: *c1ncc[nH]1] | CLM #35 | 2.6 |
| 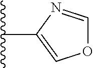 [SMILES: *c1cocn1] | CLM #36 | 7.4 |
| 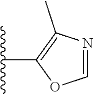 [SMILES: *c1ocnc1C] | CLM #37 | 4.8 |
| 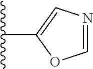 [SMILES: *c1cnco1] | CLM #38 | 5.2 |
| 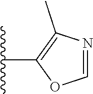 [SMILES: *c1ocnc1C] | CLM #39 | 3.2 |
| 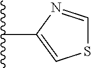 [SMILES: *c1cscn1] | CLM #40 | 7.3 |
| 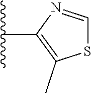 [SMILES: *c1ncsc1C] | CLM #41 | 5.5 |
| 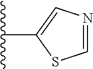 [SMILES: *c1cncs1] | CLM #42 | 4.1 |
| 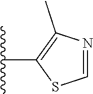 [SMILES: *c1scnc1C] | CLM #43 | 3.3 |
| 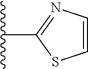 [SMILES: *c1nccs1] | CLM #44 | 6.1 |
| 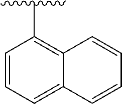 [SMILES: *c1cccc2ccccc12] | CLM #45 | 8.0 |
| 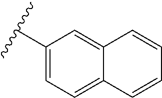 [SMILES: *c1ccc2ccccc2c1] | CLM #46 | 5.9 |
| 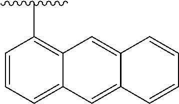 [SMILES: *c1cccc2cc3ccccc3cc12] | CLM #47 | 2.8 |
| 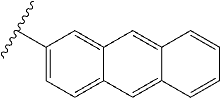 [SMILES: *c1ccc2cc3ccccc3cc2c1] | CLM #48 | 0.5 |
| 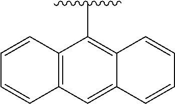 [SMILES: *c1c2ccccc2cc2ccccc12] | CLM #49 | 6.2 |
| 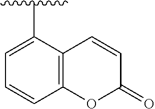 [SMILES: *c1cccc2oc(=O)ccc12] | CLM #50 | 9.2 |
| 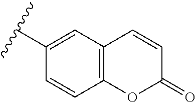 [SMILES: *c1ccc2oc(=O)ccc2c1] | CLM #51 | 6.4 |
| 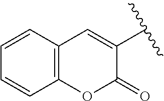 [SMILES: *c1cc2ccccc2oc1=O] | CLM #52 | 6.0 |
| 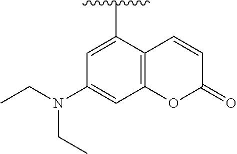 [SMILES: *c1cc(N(CC)CC)cc2oc(=O)ccc12] | CLM #53 | 10.0 |
| 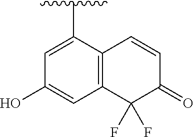 [SMILES: *c1cc(O)cc2c1C=CC(=O)C2(F)F] | CLM #54 | 10.8 |
| 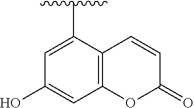 [SMILES: *c1cc(O)cc2oc(=O)ccc12] | CLM #55 | 9.8 |
| 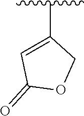 [SMILES: *C1=CC(=O)OC1] | CLM #56 | 11.2 |
| 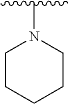 [SMILES: *N1CCCCC1] | CLM #57 | −5.6 |
| 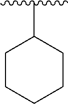 [SMILES: *C1CCCCC1] | CLM #58 | 10.6 |
| 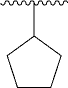 [SMILES: *C1CCCC1] | CLM #59 | 10.2 |
| 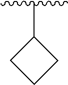 [SMILES: *C1CCC1] | CLM #60 | 10.5 |
| 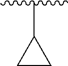 [SMILES: *C1CC1] | CLM #61 | 6.6 |
| 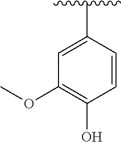 [SMILES: *c1ccc(O)c(OC)c1] | CLM #62 | 5.6 |
| 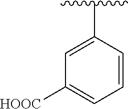 [SMILES: *c1cccc(C(=O)O)c1] | CLM #63 | 7.9 |
| 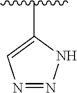 [SMILES: *c1cnn[nH]1] | CLM #64 | 5.8 |
| 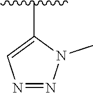 [SMILES: *c1cnnn1C] | CLM #65 | 5.1 |
| 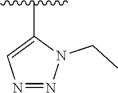 [SMILES: *c1cnnn1CC] | CLM #66 | 4.9 |
| 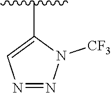 [SMILES: *c1cnnn1C(F)(F)F] | CLM #67 | 10.3 |
| 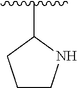 [SMILES: *C1CCCN1] | CLM #68 | 9.3 |
| 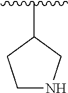 [SMILES: *C1CCNC1] | CLM #69 | 10.0 |
| 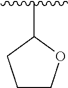 [SMILES: *C1CCCO1] | CLM #70 | 14.7 |
| 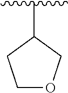 [SMILES: *C1CCOC1] | CLM #71 | 10.4 |
| 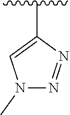 [SMILES: *c1cn(C)nn1] | CLM #72 | 8.1 |
| 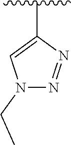 [SMILES: *c1cn(CC)nn1] | CLM #73 | 9.2 |
| 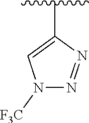 [SMILES: *c1cn(C(F)(F)F)nn1] | CLM #74 | 9.3 |
| 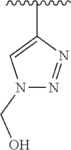 [SMILES: *c1cn(CO)nn1] | CLM #75 | 7.1 |
| 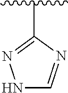 [SMILES: *c1nc[nH]n1] | CLM #76 | 9.1 |
| 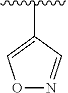 [SMILES: *c1cnoc1] | CLM #77 | 5.3 |
| 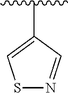 [SMILES: *c1cnsc1] | CLM #78 | 5.4 |
| 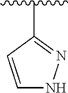 [SMILES: *c1cc[nH]n1] | CLM #79 | 4.6 |
| 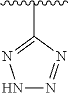 [SMILES: *c1nn[nH]n1] | CLM #80 | 10.6 |

### Example 3. Synthetic Protocols to Generate a Prodrug

Camptothecin (CPT) is a topoisomerase inhibitor derived from the bark and stem of Camptotheca acuminata (i.e., the Happy Tree). CPT showed anticancer activity in preliminary clinical trials, especially against breast, ovarian, colon, lung, and stomach cancers, by forming disrupting the function of topoisomerase I during cellular replication. As a result, the enzyme is inhibited and DNA structure is damaged, leading to apoptosis. Unfortunately, major pharmacological limitations preclude clinical application due to poor water solubility and rapid hydrolysis at physiological pH, leading to an inactive carboxylate form. These limitations make CPT a suitable candidate for developing a CPT-prodrug. Below is a synthetic scheme for deriving a prodrug containing a camptothecin moiety with a CLM, and the resultant prodrug may be further conjugated to a tumor targeting antibody as shown in Scheme 4. This protocol may be modified to develop an array of different prodrugs containing CLMs as described herein and varying combinations of different therapeutic moieties and/or antibodies. For example, FIG. 2B presents the application of the CLMs described herein to replace the linkers used in commercially available ADCs. Such utility of the CLMs described herein could facilitate overcoming limitations of current ADCs, such as undesirable generation of byproducts and premature cleavage of the linker moieties.

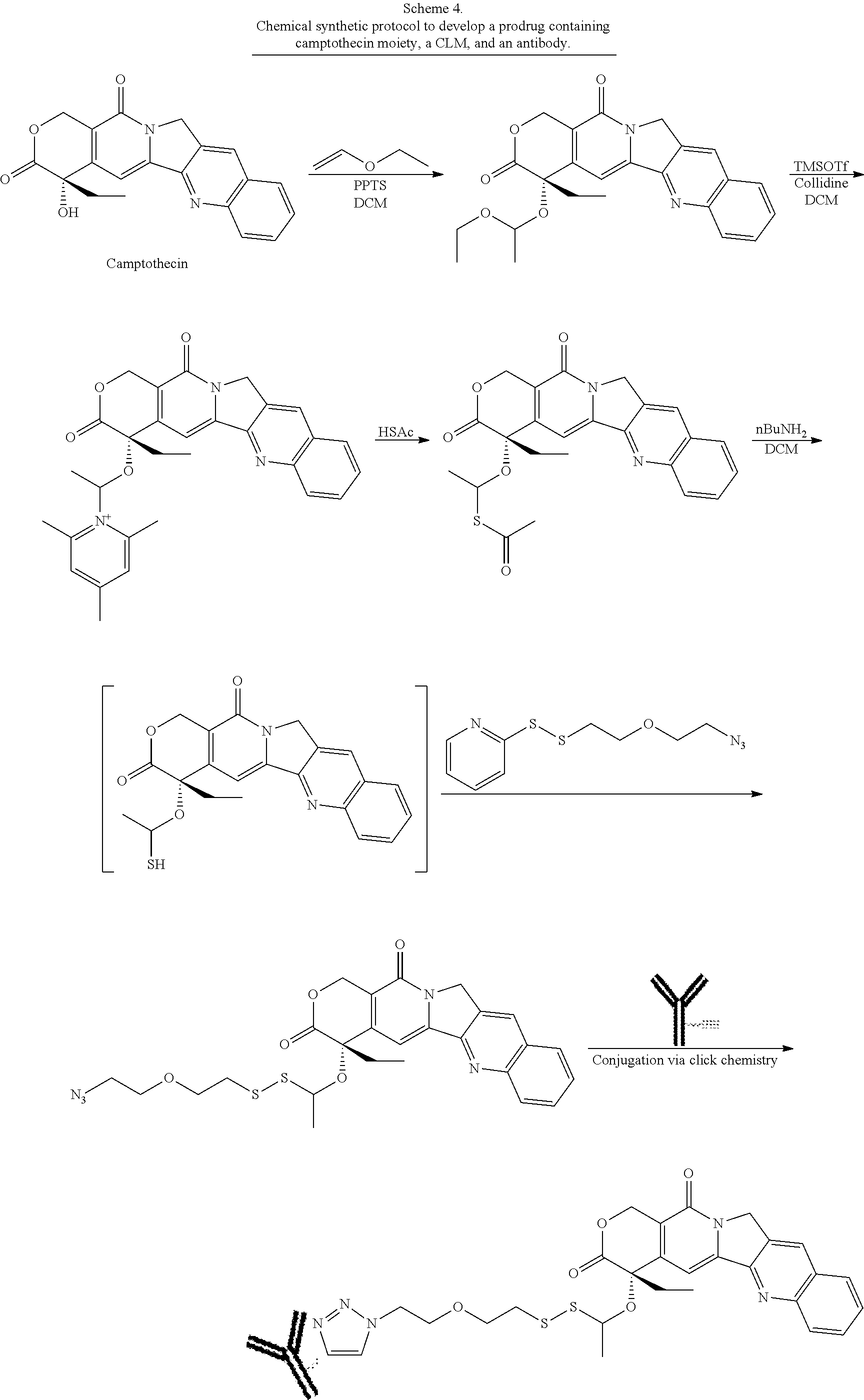

What is claimed is:

1. A prodrug having the formula: 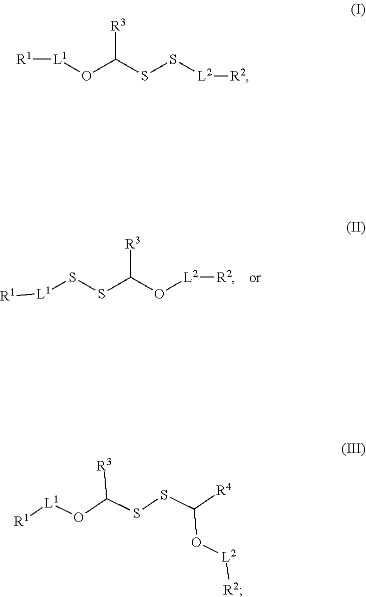
wherein,
R¹ is a first drug moiety;
R² is a second drug moiety, protein moiety, nucleic acid moiety, a polymer moiety, nanoparticle, or a detectable agent;
R³ and R⁴ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and
L¹ and L² are independently covalent linkers.

2. The prodrug of claim 1, wherein L¹ and L² are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

3. The prodrug of claim 1, wherein R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

4. The prodrug of claim 1, wherein R³ is hydrogen.

5. The prodrug of claim 1, wherein R³ is substituted or unsubstituted alkyl.

6. The prodrug of claim 1, wherein R³ is substituted or unsubstituted aryl, or substituted or unsubstituted heteroaryl.

7. The prodrug of claim 1, wherein R³ is substituted or unsubstituted aryl.

8. The prodrug of claim 1, wherein R² is a protein, wherein the protein is an antibody or antigen-binding antibody fragment.

9. The prodrug of claim 8, wherein said antibody or antibody fragment is an IgG1, IgG2a, IgG3, or IgG4 antibody or antibody fragment.

10. The prodrug of claim 8, wherein said antibody or antibody fragment is capable of specifically binding to Trop-2, CEACAM5, CEACAM6, CD19, CD20, CD22, CD25, CD30, CD33, CD37, CD38, CD46, CD56, CD71, CD74, CD79, CD123, CD166, CD205, CD228, CD276, HER2, HER3, folate receptor alpha, HLA-DR, nectin-4, tissue factor, 5T4, ADAM9, AG7, AXL, ENPP3, c-Met, EGFR, gpNMB, IGFR, integrin beta-6, mesothelin, CDH6, BCMA, NaPi2b, PSMA, ROR1, or CCR7.

11. The prodrug of claim 8, wherein said antibody or antibody fragment is capable of specifically binding to Trop-2, CEACAM5, CEACAM6, CD20, CD22, CD30, CD46, CD74, HER2, folate receptor, or HLA-DR.

12. The prodrug of claim 1, wherein R⁴ is hydrogen.

13. The prodrug of claim 1, wherein R⁴ is substituted or unsubstituted alkyl.

14. The prodrug of claim 1, wherein R⁴ is substituted or unsubstituted aryl, or substituted or unsubstituted heteroaryl.

15. The prodrug of claim 1, wherein R¹ is an antibacterial drug moiety, immunomodulator drug moiety, a photosensitizer drug moiety, or chemotherapeutic drug moiety.

16. The prodrug of claim 1, wherein R¹ is a chemotherapeutic drug moiety.

17. The prodrug of claim 1, wherein R¹ is a BCS class IV drug moiety.

18. The prodrug of claim 1, wherein R¹ and R² are independently a temozolomide moiety, carboplatin moiety, cyclophosphamide moiety, docetaxel moiety, doxorubicin moiety, gemcitabine moiety, methotrexate moiety, paclitaxel moiety, sunitinib moiety, cisplatin moiety, 5-fluorouracil moiety, 7-ethyl-10-hydroxycamptothecin (SN-38) moiety, or a combination thereof.

19. A method for diagnosing, monitoring, or treating a disease comprising administering a therapeutically effective amount of the prodrug of claim 1.
